(19) United States
(12) Patent Application Publication
(10) Pub. No.: US 2023/0226004 A1
(43) Pub. Date: Jul. 20, 2023

(54) TREATMENT OF CORONAVIRUS

(71) Applicant: AXELIA ONCOLOGY PTY LTD, Melbourne (AU)

(72) Inventors: Daphne Tsitoura, Melbourne (AU); Francesca Mercuri, Melbourne (AU); Christophe Demaison, Melbourne (AU); Nathan Bartlett, Melbourne (AU)

(21) Appl. No.: 17/926,886

(22) PCT Filed: May 26, 2021
(86) PCT No.: PCT/AU2021/050506

§ 371 Date: Nov. 21, 2022

(30) Foreign Application Priority Data
May 26, 2020 (AU) 2020901711
Jun. 26, 2020 (AU) 2020902151

(51) Int. Cl. A61K 31/223; A61K 47/60; A61P 31/14

(52) U.S. Cl. CPC A61K 31/223; A61K 47/60; A61P 31/14

(57) ABSTRACT

The present invention relates to compounds and their compositions, and the use of such compounds and compositions in the prevention and/or treatment of respiratory infections, or diseases or conditions associated with coronavirus infections. The compositions comprise therapeutically effective amounts of a TLR2 agonist. Certain embodiments specify that the TLR2 agonist is a pegylated, palmitoylated-cysteine compound of Formula (I), (VI), (VII) or (VIII).

### FIELD OF THE INVENTION

The present invention relates to compounds and their compositions, and the use of such compounds and compositions in the prevention and/or treatment of coronavirus infections, or respiratory diseases or conditions associated with coronavirus infections.

### CROSS-REFERENCE TO RELATED APPLICATIONS

This patent application claims priority from Australian provisional applications 2020901711 and 2020902151 the entire contents of each are herein incorporated by reference in their entirety.

### BACKGROUND OF THE INVENTION

Emerging respiratory coronaviruses offer a considerable threat to the health of global populations and the economy. Coronaviruses (CoVs) constitute a group of phylogenetically diverse enveloped viruses that encode the largest plus strand RNA genomes and replicate efficiently in most mammals. Human CoV (HCoVs-229E, OC43, NL63, and HKU1) infections typically result in mild to severe upper and lower respiratory tract disease.

Coronaviruses, belong to the Coronaviridae family in the Nidovirales order, are minute in size (65-125 nm in diameter) and contain a single-stranded RNA as a nucleic material, size ranging from 26 to 32 kbs in length. The subgroups of coronaviruses family are alpha (α), beta (β), gamma (γ) and delta (δ) coronavirus. The severe acute respiratory syndrome coronavirus (SARS-CoV) and Middle East respiratory syndrome coronavirus (MERS-CoV) cause acute lung injury (ALI) and acute respiratory distress syndrome (ARDS) which leads to pulmonary failure and result in fatality.

Severe Acute Respiratory Syndrome Coronavirus (SARS-CoV) emerged in 2002-2003 causing acute respiratory distress syndrome (ARDS) with 10% mortality overall and up to 50% mortality in aged individuals.

Middle Eastern Respiratory Syndrome Coronavirus (MERS-CoV) emerged in the Middle East in April of 2012, manifesting as severe pneumonia, acute respiratory distress syndrome (ARDS) and acute renal failure.

In 2020, the world is faced with an extreme situation of a highly infectious coronavirus (2019-nCoV; SARS-CoV2) encountered by a global immunologically naïve population, manifesting as a disease termed “COVID-19”. SARS-CoV2 infections globally have exceeded 2 million confirmed cases with more than 150,000 deaths to date. COVID-19 manifestations range from mild to severe life-threatening with a substantial mortality rate.

There is a need for new or improved treatments for coronavirus infections and/or diseases associated with, or caused by, coronaviruses.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides compounds useful in treating and/or preventing coronavirus infections and respiratory diseases or conditions associated with coronavirus infections.

In one aspect, the present invention provides a method for reducing a coronavirus infection in a subject, the method comprising administering to the subject a therapeutically effective amount of a TLR2 agonist, preferably an agonist of a heterodimer of TLR2 and TLR6, thereby reducing the coronavirus infection in the subject.

The TLR2 agonist may be any of the TLR2 agonists described herein. Preferably, the TLR2 agonist is a compound as defined by any one of formulas (I), (IA1), (IA2), (II), (III), (IV), (V), (VI), (VII), (VIII), (IX), (X), (XI), (XII), (XIII), (XIV), (XV), (XVI), (XVII), (XVIII) and (XIX) (collectively referred to herein as formulas (I)-(XIX)).

In any aspect of the invention, the TLR2 agonist may be a compound comprising moiety A selected from A1′ and A2 as defined herein and a polyethylene glycol (PEG), wherein the moiety A and PEG are linked by a glycine, serine, homoserine, threonine, phosphoserine, asparagine or glutamine residue, or an ester of a glutamine residue.

In any aspect of the invention, the compound may comprise or consist of partial structure A1Y′ or A2Y′:

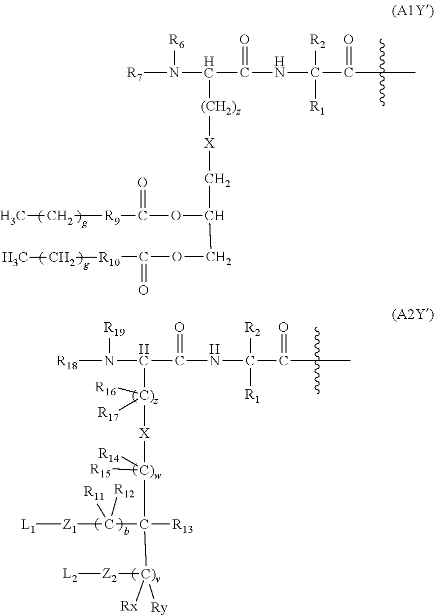

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are independently selected from the group consisting of H, a straight or branched C₁-C₄ alkyl, and —C(═O)CH₃;

R₈ is selected from the group consisting of H and a straight or branched C₁-C₆ alkyl;

R₉ and R₁₀ are independently selected from the group consisting of —NH—, —O— or a single bond;

z is 1 or 2;

X is selected from —S—, —S(═O)— and —S(═O)₂—;

b and w are each independently an integer from 0 to 7 and v is an integer from 0 to 5, provided that:

the sum of b, v, and w is at least 3; and

the sum of b and w is from 0 to 7;

Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;

R₁₁, R₁₂, Rₓ, R_{y}, R₁₄, R₁₅, R₁₆, and R₁₇ at each instance of b, v, w, and z are each independently H or C₁-C₆ aliphatic;

R, R₁₃ and R₁₈ are each independently H or C₁-C₆ aliphatic;

R₁₉ is H, C₁-C₆ aliphatic, an amino protecting group, L₃-C(═O)—, or A2;

L₁ and L₂ are each independently C₅-C₂₁ aliphatic or C₄-C₂₀ heteroaliphatic;

L₃ is C₁-C₂₁ aliphatic or C₂-C₂₀ heteroaliphatic;

A2 is an amino acid or a peptide;

wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted; and

A1Y′ or A2Y′ is covalently linked to polyethylene glycol (PEG),

or a pharmaceutically acceptable salt, solvate or prodrug thereof.

In some embodiments, the TLR2 agonist is a compound selected from any of compounds 001-010, A101-A114 and A201-A232.

Reference to a “compound of the invention” as used herein may refer to any of:
a compound of formulas (I)-(XIX);
a compound selected from any of compounds 001-010, A101-A114 and A201-A232;
a compound comprising moiety A selected from A1′ and A2 as defined herein and a polyethylene glycol (PEG), wherein the moiety A and PEG are linked by a glycine, serine, homoserine, threonine, phosphoserine, asparagine or glutamine residue, or an ester of a glutamine residue; and/or
a compound comprising a partial structure of formula (A1Y′) or (A2Y′) covalently linked to a PEG.

The present invention also provides for compositions comprising, consisting essentially of, or consisting of, a compound of the invention as described herein or a pharmaceutically acceptable salt, solvate or prodrug thereof, and a pharmaceutically acceptable carrier, diluent or excipient.

In one aspect, the present invention provides a method for reducing a coronavirus infection in a subject, the method comprising administering to the subject a therapeutically effective amount of a compound of the invention as described herein, thereby reducing the coronavirus infection in the subject.

In another aspect, the present invention provides a method of treating and/or preventing a disease associated with a coronavirus, the method comprising raising an innate immune response in a subject by administering to the subject in need thereof a compound of the invention as described herein or a pharmaceutically acceptable salt, solvate or prodrug thereof, or pharmaceutical composition as described herein, thereby treating and/or preventing a disease associated with a coronavirus.

In another aspect, the present invention provides a method of treating and/or preventing a disease associated with, or caused by, a coronavirus, the method comprising administering to a subject in need thereof a compound of the invention as described herein or a pharmaceutically acceptable salt, solvate or prodrug thereof, or pharmaceutical composition as described herein, thereby treating and/or preventing a disease associated with, or caused by, a coronavirus.

In another aspect, the present invention provides a method of treating and/or preventing a respiratory disease or condition associated with a coronavirus infection, the method comprising administering to a subject in need thereof a compound of the invention as described herein or a pharmaceutically acceptable salt, solvate or prodrug thereof, or pharmaceutical composition as described herein, thereby treating and/or preventing a respiratory disease or condition associated with a coronavirus infection.

In another aspect, the present invention provides a method for reducing airway inflammation associated with, or caused by, a coronavirus, the method comprising administering to a subject in need thereof a compound of the invention as described herein or a pharmaceutically acceptable salt, solvate or prodrug thereof, or pharmaceutical composition as described herein, thereby reducing airway inflammation associated with, or caused by, a coronavirus.

In another aspect, the present invention also provides a method of improving the ability of a subject to control a respiratory disease or condition during a coronavirus infection, the method comprising administering to a subject in need thereof a compound of the invention as described herein or a pharmaceutically acceptable salt, solvate or prodrug thereof, or pharmaceutical composition as described herein, thereby improving the ability of a subject to control a respiratory disease or condition during a coronavirus infection.

In another aspect, the present invention provides for use of a compound of the invention as described herein or a pharmaceutically acceptable salt, solvate or prodrug thereof, or pharmaceutical composition as described herein, in the preparation of a medicament for raising an innate immune response in a subject diagnosed with, or suspected of having, a coronavirus infection.

In another aspect, the present invention provides for use of a compound of the invention as described herein or a pharmaceutically acceptable salt, solvate or prodrug thereof, or pharmaceutical composition as described herein, in the preparation of a medicament for treating and/or preventing a disease caused by a coronavirus.

In another aspect, the present invention further provides for use of a compound of the invention as described herein or a pharmaceutically acceptable salt, solvate or prodrug thereof, or pharmaceutical composition as described herein, in the preparation of a medicament for treating and/or preventing a respiratory disease or condition associated with a coronavirus infection in a subject.

In another aspect, the present invention further provides for use of a compound of the invention as described herein or a pharmaceutically acceptable salt, solvate or prodrug thereof, or pharmaceutical composition as described herein, in the preparation of a medicament for treating and/or preventing a coronavirus infection in a subject.

In another aspect, the present invention further provides use of a compound of the invention as described herein or a pharmaceutically acceptable salt, solvate or prodrug thereof, or pharmaceutical composition as described herein, in the preparation of a medicament for reducing airway inflammation in a subject diagnosed with, or suspected of having, a coronavirus infection.

In another aspect, the present invention further provides use of a compound of the invention as described herein or a pharmaceutically acceptable salt, solvate or prodrug thereof, or pharmaceutical composition as described herein, in the preparation of a medicament for improving the ability of a subject to control a respiratory disease or condition during a coronavirus infection.

In one aspect, the present invention provides for a compound of the invention as described herein or a pharmaceutically acceptable salt, solvate or prodrug thereof, or pharmaceutical composition as described herein, for use in raising an innate immune response in a subject diagnosed with, or suspected of having, a coronavirus infection.

In another aspect, the present invention provides for a compound of the invention as described herein or a pharmaceutically acceptable salt, solvate or prodrug thereof, or pharmaceutical composition as described herein, for use in treating and/or preventing a disease caused by a coronavirus in a subject.

In another aspect, the present invention provides for a compound of the invention as described herein or a pharmaceutically acceptable salt, solvate or prodrug thereof, or pharmaceutical composition as described herein, for use in treating and/or preventing a respiratory disease or condition associated with a coronavirus infection in a subject.

In another aspect, the invention provides a compound of the invention as described herein or a pharmaceutically acceptable salt, solvate or prodrug thereof, or pharmaceutical composition as described herein, for use in reducing airway inflammation in a subject diagnosed with, or suspected of having, a coronavirus infection.

In another aspect, the invention provides a compound of the invention as described herein or a pharmaceutically acceptable salt, solvate or prodrug thereof, or pharmaceutical composition as described herein, for use in controlling a respiratory disease or condition during a coronavirus infection in a subject.

The present invention also provides a kit for use, or when used, in a method of the invention, the kit comprising, consisting essentially of or consisting of:
a compound of the invention as described herein or a pharmaceutically acceptable salt, solvate or prodrug thereof, or pharmaceutical composition as described herein; and optionally
written instructions describing the use of the compound in a method of the invention.

In any aspect of the present invention, the coronavirus may be from any of the genera Alphacoronavirus, Betacoronavirus, Gammacoronavirus or Deltacoronavirus. Preferably, the coronavirus is from one of the Alphacoronavirus subgroup clusters 1a and 1 b or one of the Betacoronavirus subgroup clusters 2a, 2b, 2c, and 2d. The coronavirus may be any one that infects humans. Exemplary coronaviruses are SARS-CoV, MERS-CoV, SARS-CoV2, HCoV-NL63, HCoV-229E, HCoV-OC43 and HKU1, although the coronavirus may be any one as described herein. Most preferably, the coronavirus is SARS-CoV2.

In any aspect of the invention, the TLR2 agonist is not administered with an antigen. In another aspect, the TLR2 agonist is not administered with a cell penetrating peptide.

In any aspect of the invention, the TLR2 agonist is not Pam3Cys.

In any embodiment of the invention, the method does not comprise administering agonists of TLRs other than TLR2 homodimers or heterodimers.

In any aspect of the invention, the TLR2 agonist may be conjugated with other compounds or functional groups. Other compounds or functional groups are any of those described herein. Preferred compounds are selected on the basis to assist in dissolving the TLR2 agonist in a carrier, diluent, excipient or solvent.

Depending on the polarity of the solvent, the solubility of the TLR2 agonist may be increased by a solubilising agent. Therefore, the compound may comprise a TLR2 agonist and a solubilising agent. Preferably, the TLR2 agonist and solubilising agent are linked. The TLR2 agonist may be PEGylated. Preferably, the solubilising agent is any molecule as described herein.

The solubilising agent may comprise, consist essentially, or consist of a positively or negatively charged group. Preferably, the charged group is a branched or linear peptide. Preferably, the positively charged group comprises at least one positively charged amino acid, such as an arginine or lysine residue. Preferably, the negatively charged group comprises at least one negatively charged amino acid, such as glutamate or aspartate. The charged amino acids may be terminal, preferably N-terminal.

Typically, the solubilising agent comprises polyethyleneglycol (PEG) or R4. In any aspect of the invention, the solubilising agent comprises polyethyleneglycol (PEG) and R4.

In any aspect of the invention, the compound comprises Pam2Cys conjugated to PEG₁₁. In any aspect of the invention, the Pam2Cys and PEG₁₁ molecules are separated by at least one serine.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Human TLR2 activity of (a) compounds A107, A108, A115, A116, A203, A204, A215 and A216 from the NK-κB luciferase assay described in Example 2.1; and (b) Human TLR2 activity of compounds A108, A220 and A224 from the assay described in Example 2.2 (L to R, A108 first column, A220 second column, A224 third column, blank control final column).

FIG. 2. Individual % body weight change from baseline for animals in the 4 groups (3 treatment groups and control)±SEM.

FIG. 3. Mean % body weight change from baseline for animals in the 4 groups (3 treatment groups and control). Kruskal-Wallis test on area under curve showed no significant difference between groups pre- or post-challenge.

FIG. 4. Mean temperature (° C.) of animals in the 4 groups (3 treatment groups and control). Kruskal-Wallis test on area under curve showed no significant difference between groups post-challenge.

FIG. 5. Viral RNA measures as genome copies/ml in nasal wash over time post-challenge for 4 groups (3 treatment groups and control) (a) displaying results of individual samples, (b) displaying geometric mean±geometric SD. LOQ=limit of quantitation.

FIG. 6. Viral RNA measures as genome copies/ml in throat swabs over time post-challenge for 4 groups (3 treatment groups and control) (a) displaying results of individual samples, (b) displaying geometric mean±geometric SD. LOQ=limit of quantitation.

FIG. 7. A comparison of treated vs untreated groups based on throat swab PCR data (a) or nasal wash PCT data (b). Using a two-way ANOVA (Sidak's multiple comparison test) of individual logged data for treated (groups 1-3 combined) vs untreated (group 4), there were significant differences at throat swab day 5 and 7, p=<0.0001, and nasal wash day 5, p=0.0073.

FIG. 8. Upper respiratory A101 treatment primes lymphocyte recruitment in BAL following upper respiratory coronavirus (OC43) infection. Bronchoalveolar lavage (BAL/lower airway) cells were differentially stained and counted by light microscopy. Data show Mean+/−SEM *p<0.05, **p<0.01 by 2-way ANOVA (mixed effects model with Dunnett's correction for multiple comparisons), n=7-8. Total BAL cells shown in (a) and total BAL lymphocytes shown in (b).

FIG. 9. Upper respiratory A101 treatment primes enhanced antiviral gene expression following upper respiratory coronavirus (OC43) infection. Nasal turbinates were collected at day 0, 1, 3 and 5 days post-infection, total RNA extracted and differential expression of IFN-A (a), Viperine (b), PKR (c) and OAS (d) was determined by TaqMan qPCR. Mean+/−SEM. *p<0.05, ***p<0.001, ****p<0.0001 by 2-way ANOVA (mixed effects model with Dunnett's correction for multiple comparisons), n=7-8. Figure legend for all parts of FIG. 9 is shown in FIG. 9(d).

FIG. 10. URT A101 treatment significantly altered expression of OC43 viral RNA. Nasal turbinates and nasal wash were collected on day 0, 2 hours post-infection. Total RNA was extracted from tissue samples and viral RNA was purified from nasal wash. OC43 viral RNA measured by TaqMan qPCR. Data represents Mean+/−SEM-URT Saline OC43 left column, and URT d-7, d-3 A101 OC43 right column. **p<0.01 by 2-way ANOVA (with Dunnett's correction for multiple comparisons), n=8.

FIG. 11. (a) Weight loss of hamsters treated with A204 or PBS prior to infection and/or 8 hours post infection. Hamsters were intranasally treated with 50 ug/ml in 100 ul PBS 24 hours prior to infection or 8 hours post infection with 5×10⁴ PFU SARS-CoV-2 in 100 ul PBS. (b) RT-qPCR data quantifying lung viral load as assessed by N RNA copy number per ug total lung RNA.

FIG. 12. Weight loss of hamsters treated with 10 ug/ml A204 in 100 ul or PBS after infection with SARS-Cov-2. Hamsters were intranasally infected with 10⁴ PFU SARS-CoV-2 in 100 ul PBS and treated with A204 or PBS 8 hours post infection. Weights were measured 7-Days post infection.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

As used herein, except where the context requires otherwise, the term “comprise” and variations of the term, such as “comprising”, “comprises” and “comprised”, are not intended to exclude further additives, components, integers or steps.

For purposes of interpreting this specification, terms used in the singular will also include the plural and vice versa.

The general chemical terms used in the formulae herein have their usual meaning.

The present inventors have surprisingly identified compounds that are useful in reducing coronavirus infection and can be used to treating diseases or conditions associated with, or caused by, coronavirus infection.

Coronavirus

“Coronavirus” as used herein refers members of the subfamily Coronavirinae in the family Coronaviridae and the order Nidovirales (International Committee on Taxonomy of Viruses). This subfamily consists of four genera, Alphacoronavirus, Betacoronavirus, Gammacoronavirus and Deltacoronavirus, on the basis of their phylogenetic relationships and genomic structures. Subgroup clusters are labeled as 1a and 1b for the Alphacoronavirus and 2a, 2b, 2c, and 2d for the Betacoronavirus. The alphacoronaviruses and betacoronaviruses infect only mammals. The gammacoronaviruses and deltacoronaviruses infect birds, but some of them can also infect mammals. Alphacoronaviruses and betacoronaviruses usually cause respiratory illness in humans and gastroenteritis in animals. The three highly pathogenic viruses, SARS-CoV, MERS-CoV and SARS-CoV2, cause severe respiratory syndrome in humans, and the other four human coronaviruses (HCoV-NL63, HCoV-229E, HCoV-OC43 and HKU1) induce only mild upper respiratory diseases in immunocompetent hosts, although some of them can cause severe infections in infants, young children and elderly individuals. Alphacoronaviruses and betacoronaviruses can pose a heavy disease burden on livestock; these viruses include porcine transmissible gastroenteritis virus, porcine enteric diarrhoea virus (PEDV) and the recently emerged swine acute diarrhoea syndrome coronavirus (SADS-CoV). On the basis of current sequence databases, all human coronaviruses have animal origins: SARS-CoV, MERS-CoV, SARS-CoV2, HCoV-NL63 and HCoV-229E are considered to have originated in bats; HCoV-OC43 and HKU1 likely originated from rodents.

The coronaviruses include antigenic groups I, II, and Ill. Nonlimiting examples of coronaviruses include SARS coronavirus, MERS coronavirus, transmissible gastroenteritis virus (TGEV), human respiratory coronavirus, porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus, as well as any others described herein, and including those referred to in Cui, et al. Nature Reviews Microbiology volume 17, pages 181-192 (2019), and Shereen et al. Journal of Advanced Research, Volume 24, July 2020 (published online 16 Mar. 2020), Pages 91-98.

Nonlimiting examples of a subgroup 1a coronavirus include FCov.FIPV.79.1146.VR.2202 (GenBank Accession No. NV_007025), transmissible gastroenteritis virus (TGEV) (GenBank Accession No. NC_002306; GenBank Accession No. Q811789.2; GenBank Accession No. DQ811786.2; GenBank Accession No. DQ811788.1; GenBank Accession No. DQ811785.1; GenBank Accession No. X52157.1; GenBank Accession No. AJ011482.1; GenBank Accession No. KC962433.1; GenBank Accession No. AJ271965.2; GenBank Accession No. JQ693060.1; GenBank Accession No. KC609371.1; GenBank Accession No. JQ693060.1; GenBank Accession No. JQ693059.1; GenBank Accession No. JQ693058.1; GenBank Accession No. JQ693057.1; GenBank Accession No. JQ693052.1; GenBank Accession No. JQ693051.1; GenBank Accession No. JQ693050.1), porcine reproductive and respiratory syndrome virus (PRRSV) (GenBank Accession No. NC_001961.1; GenBank Accession No. DQ811787), as well as any other subgroup 1a coronavirus now known (e.g., as can be found in the GenBank® Database) or later identified, and any combination thereof.

Nonlimiting examples of a subgroup 1b coronavirus include BtCoV.1A.AFCD62 (GenBank Accession No. NC_010437), BtCoV.1B.AFCD307 (GenBank Accession No. NC_010436), BtCov.HKU8.AFCD77 (GenBank Accession No. NC_010438), BtCoV.512.2005 (GenBank Accession No. DQ648858), porcine epidemic diarrhea virus PEDV.CV777 (GenBank Accession No. NC_003436, GenBank Accession No. DQ355224.1, GenBank Accession No. DQ355223.1, GenBank Accession No. DQ355221.1, GenBank Accession No. JN601062.1, GenBank Accession No. N601061.1, GenBank Accession No. JN601060.1, GenBank Accession No. JN601059.1, GenBank Accession No. JN601058.1, GenBank Accession No. JN601057.1, GenBank Accession No. JN601056.1, GenBank Accession No. JN601055.1, GenBank Accession No. JN601054.1, GenBank Accession No. JN601053.1, GenBank Accession No. JN601052.1, GenBank Accession No. JN400902.1, GenBank Accession No. JN547395.1, GenBank Accession No. FJ687473.1, GenBank Accession No. FJ687472.1, GenBank Accession No. FJ687471.1, GenBank Accession No. FJ687470.1, GenBank Accession No. FJ687469.1, GenBank Accession No. FJ687468.1, GenBank Accession No. FJ687467.1, GenBank Accession No. FJ687466.1, GenBank Accession No. FJ687465.1, GenBank Accession No. FJ687464.1, GenBank Accession No. FJ687463.1, GenBank Accession No. FJ687462.1, GenBank Accession No. FJ687461.1, GenBank Accession No. FJ687460.1, GenBank Accession No. FJ687459.1, GenBank Accession No. FJ687458.1, GenBank Accession No. FJ687457.1, GenBank Accession No. FJ687456.1, GenBank Accession No. FJ687455.1, GenBank Accession No. FJ687454.1, GenBank Accession No. FJ687453 GenBank Accession No. FJ687452.1, GenBank Accession No. FJ687451.1, GenBank Accession No. FJ687450.1, GenBank Accession No. FJ687449.1, GenBank Accession No. AF500215.1, GenBank Accession No. KF476061.1, GenBank Accession No. KF476060.1, GenBank Accession No. KF476059.1, GenBank Accession No. KF476058.1, GenBank Accession No. KF476057.1, GenBank Accession No. KF476056.1, GenBank Accession No. KF476055.1, GenBank Accession No. KF476054.1, GenBank Accession No. KF476053.1, GenBank Accession No. KF476052.1, GenBank Accession No. KF476051.1, GenBank Accession No. KF476050.1, GenBank Accession No. KF476049.1, GenBank Accession No. KF476048.1, GenBank Accession No. KF177258.1, GenBank Accession No. KF177257.1, GenBank Accession No. KF177256.1, GenBank Accession No. KF177255.1), HCoV.229E (GenBank Accession No. NC_002645), HCoV.NL63.Amsterdam.1 (GenBank Accession No. NC_005831), BtCoV.HKU2.HK.298.2006 (GenBank Accession No. EF203066), BtCoV.HKU2.HK.33.2006 (GenBank Accession No. EF203067), BtCoV.HKU2.HK.46.2006 (GenBank Accession No. EF203065), BtCoV.HKU2.GD.430.2006 (GenBank Accession No. EF203064), as well as any other subgroup 1b coronavirus now known (e.g., as can be found in the GenBank® Database) or later identified, and any combination thereof.

Nonlimiting examples of a subgroup 2a coronavirus include HCoV.HKU1.C.N5 (GenBank Accession No. DQ339101), MHV.A59 (GenBank Accession No. NC 001846), PHEV.VW572 (GenBank Accession No. NC 007732), HCoV.OC43.ATCC.VR.759 (GenBank Accession No. NC_005147), bovine enteric coronavirus (BCoV.ENT) (GenBank Accession No. NC_003045), as well as any other subgroup 2a coronavirus now known (e.g., as can be found in the GenBank® Database) or later identified, and any combination thereof.

Nonlimiting examples of subgroup 2b coronaviruses include Bat SARS CoV (GenBank Accession No. FJ211859), SARS CoV (GenBank Accession No. FJ211860), SARS-CoV2 (GenBank Accession No. NC_045512.2), BtSARS.HKU3.1 (GenBank Accession No. DQ022305), BtSARS.HKU3.2 (GenBank Accession No. DQ084199), BtSARS.HKU3.3 (GenBank Accession No. DQ084200), BtSARS.Rm1 (GenBank Accession No. DQ412043), BtCoV.279.2005 (GenBank Accession No. DQ648857), BtSARS.Rf1 (GenBank Accession No. DQ412042), BtCoV.273.2005 (GenBank Accession No. DQ648856), BtSARS.Rp3 (GenBank Accession No. DQ071615), SARS CoV.A022 (GenBank Accession No. AY686863), SARSCoV.CUHK-W1 (GenBank Accession No. AY278554), SARSCoV.GDO1 (GenBank Accession No. AY278489), SARSCoV.HC.SZ.61.03 (GenBank Accession No. AY515512), SARSCoV.SZ16 (GenBank Accession No. AY304488), SARSCoV.Urbani (GenBank Accession No. AY278741), SARSCoV.civet010 (GenBank Accession No. AY572035), and SARSCoV.MA.15 (GenBank Accession No. DQ497008), Rs SHCO14 (GenBank® Accession No. KC881005), Rs3367 (GenBank® Accession No. KC881006), WiV1 S (GenBank® Accession No. KC881007) as well as any other subgroup 2b coronavirus now known (e.g., as can be found in the GenBank® Database) or later identified, and any combination thereof.

Nonlimiting examples of subgroup 2c coronaviruses include: Middle East respiratory syndrome coronavirus isolate Riyadh_2_2012 (GenBank Accession No. KF600652.1), Middle East respiratory syndrome coronavirus isolate Al-Hasa_18_2013 (GenBank Accession No. KF600651.1), Middle East respiratory syndrome coronavirus isolate Al-Hasa_172013 (GenBank Accession No. KF600647.1), Middle East respiratory syndrome coronavirus isolate Al-Hasa_15_2013 (GenBank Accession No. KF600645.1), Middle East respiratory syndrome coronavirus isolate Al-Hasa_16_2013 (GenBank Accession No. KF600644.1), Middle East respiratory syndrome coronavirus isolate Al-Hasa_21_2013 (GenBank Accession No. KF600634), Middle East respiratory syndrome coronavirus isolate Al-Hasa_19_2013 (GenBank Accession No. KF600632), Middle East respiratory syndrome coronavirus isolate Buraidah_1_2013 (GenBank Accession No. KF600630.1), Middle East respiratory syndrome coronavirus isolate Hafr-Al-Batin_1_2013 (GenBank Accession No. KF600628.1), Middle East respiratory syndrome coronavirus isolate Al-Hasa_12_2013 (GenBank Accession No. KF600627.1), Middle East respiratory syndrome coronavirus isolate Bisha_1_2012 (GenBank Accession No. KF600620.1), Middle East respiratory syndrome coronavirus isolate Riyadh_3_2013 (GenBank Accession No. KF600613.1), Middle East respiratory syndrome coronavirus isolate Riyadh_1_2012 (GenBank Accession No. KF600612.1), Middle East respiratory syndrome coronavirus isolate Al-Hasa_3_2013 (GenBank Accession No. KF186565.1), Middle East respiratory syndrome coronavirus isolate Al-Hasa_1_2013 (GenBank Accession No. KF186567.1), Middle East respiratory syndrome coronavirus isolate Al-Hasa_2_2013 (GenBank Accession No. KF186566.1), Middle East respiratory syndrome coronavirus isolate Al-Hasa_4_2013 (GenBank Accession No. KF186564.1), Middle East respiratory syndrome coronavirus (GenBank Accession No. KF192507.1), Betacoronavirus England 1-N1 (GenBank Accession No. NC_019843), MERS-CoV_SA-N1 (GenBank Accession No. KC667074), following isolates of Middle East Respiratory Syndrome Coronavirus (GenBank Accession No: KF600656.1, GenBank Accession No: KF600655.1, GenBank Accession No: KF600654.1, GenBank Accession No: KF600649.1, GenBank Accession No: KF600648.1, GenBank Accession No: KF600646.1, GenBank Accession No: KF600643.1, GenBank Accession No: KF600642.1, GenBank Accession No: KF600640.1, GenBank Accession No: KF600639.1, GenBank Accession No: KF600638.1, GenBank Accession No: KF600637.1, GenBank Accession No: KF600636.1, GenBank Accession No: KF600635.1, GenBank Accession No: KF600631.1, GenBank Accession No: KF600626.1, GenBank Accession No: KF600625.1, GenBank Accession No: KF600624.1, GenBank Accession No: KF600623.1, GenBank Accession No: KF600622.1, GenBank Accession No: KF600621.1, GenBank Accession No: KF600619.1, GenBank Accession No: KF600618.1, GenBank Accession No: KF600616.1, GenBank Accession No: KF600615.1, GenBank Accession No: KF600614.1, GenBank Accession No: KF600641.1, GenBank Accession No: KF600633.1, GenBank Accession No: KF600629.1, GenBank Accession No: KF600617.1), Coronavirus Neoromicia/PML-PHE1/RSA/2011 GenBank Accession: KC869678.2, Bat Coronavirus Taper/CII_KSA_287/Bisha/Saudi Arabia/GenBank Accession No: KF493885.1, Bat coronavirus Rhhar/CII_KSA_003/Bisha/Saudi Arabia/2013 GenBank Accession No: KF493888.1, Bat coronavirus Pikuh/CII_KSA_001/Riyadh/Saudi Arabia/2013 GenBank Accession No: KF493887.1, Bat coronavirus Rhhar/CII_KSA_002/Bisha/Saudi Arabia/2013 GenBank Accession No: KF493886.1, Bat Coronavirus Rhhar/CII_KSA_004/Bisha/Saudi Arabia/2013 GenBank Accession No: KF493884.1, BtCoV.HKU4.2 (GenBank Accession No. EF065506), BtCoV.HKU4.1 (GenBank Accession No. NC_009019), BtCoV.HKU4.3 (GenBank Accession No. EF065507), BtCoV.HKU4.4 (GenBank Accession No. EF065508), BtCoV 133.2005 (GenBank Accession No. NC 008315), BtCoV.HKU5.5 (GenBank Accession No. EF065512); BtCoV.HKU5.1 (GenBank Accession No. NC_009020), BtCoV.HKU5.2 (GenBank Accession No. EF065510), BtCoV.HKU5.3 (GenBank Accession No. EF065511), human betacoronavirus 2c Jordan-N3/2012 (GenBank Accession No. KC776174.1; human betacoronavirus 2c EMC/2012 (GenBank Accession No. JX869059.2), Pipistrellus bat coronavirus HKU5 isolates (GenBank Accession No: KC522089.1, GenBank Accession No: KC522088.1, GenBank Accession No: KC522087.1, GenBank Accession No: KC522086.1, GenBank Accession No: KC522085.1, GenBank Accession No: KC522084.1, GenBank Accession No: KC522083.1, GenBank Accession No: KC522082.1, GenBank Accession No: KC522081.1, GenBank Accession No: KC522080.1, GenBank Accession No: KC522079.1, GenBank Accession No: KC522078.1, GenBank Accession No: KC522077.1, GenBank Accession No: KC522076.1, GenBank Accession No: KC522075.1, GenBank Accession No: KC522104.1, GenBank Accession No: KC522104.1, GenBank Accession No: KC522103.1, GenBank Accession No: KC522102.1, GenBank Accession No: KC522101.1, GenBank Accession No: KC522100.1, GenBank Accession No: KC522099.1, GenBank Accession No: KC522098.1, GenBank Accession No: KC522097.1, GenBank Accession No: KC522096.1, GenBank Accession No: KC522095.1, GenBank Accession No: KC522094.1, GenBank Accession No: KC522093.1, GenBank Accession No: KC522092.1, GenBank Accession No: KC522091.1, GenBank Accession No: KC522090.1, GenBank Accession No: KC522119.1 GenBank Accession No: KC522118.1 GenBank Accession No: KC522117.1 GenBank Accession No: KC522116.1 GenBank Accession No: KC522115.1 GenBank Accession No: KC522114.1 GenBank Accession No: KC522113.1 GenBank Accession No: KC522112.1 GenBank Accession No: KC522111.1 GenBank Accession No: KC522110.1 GenBank Accession No: KC522109.1 GenBank Accession No: KC522108.1, GenBank Accession No: KC522107.1, GenBank Accession No: KC522106.1, GenBank Accession No: KC522105.1) Pipistrellus bat coronavirus HKU4 isolates (GenBank Accession No: KC522048.1, GenBank Accession No: KC522047.1, GenBank Accession No: KC522046.1, GenBank Accession No: KC522045.1, GenBank Accession No: KC522044.1, GenBank Accession No: KC522043.1, GenBank Accession No: KC522042.1, GenBank Accession No: KC522041.1, GenBank Accession No: KC522040.1 GenBank Accession No: KC522039.1, GenBank Accession No: KC522038.1, GenBank Accession No: KC522037.1, GenBank Accession No: KC522036.1, GenBank Accession No: KC522048.1 GenBank Accession No: KC522047.1 GenBank Accession No: KC522046.1 GenBank Accession No: KC522045.1 GenBank Accession No: KC522044.1 GenBank Accession No: KC522043.1 GenBank Accession No: KC522042.1 GenBank Accession No: KC522041.1 GenBank Accession No: KC522040.1, GenBank Accession No: KC522039.1 GenBank Accession No: KC522038.1 GenBank Accession No: KC522037.1 GenBank Accession No: KC522036.1, GenBank Accession No: KC522061.1 GenBank Accession No: KC522060.1 GenBank Accession No: KC522059.1 GenBank Accession No: KC522058.1 GenBank Accession No: KC522057.1 GenBank Accession No: KC522056.1 GenBank Accession No: KC522055.1 GenBank Accession No: KC522054.1 GenBank Accession No: KC522053.1 GenBank Accession No: KC522052.1 GenBank Accession No: KC522051.1 GenBank Accession No: KC522050.1 GenBank Accession No: KC522049.1 GenBank Accession No: KC522074.1, GenBank Accession No: KC522073.1 GenBank Accession No: KC522072.1 GenBank Accession No: KC522071.1 GenBank Accession No: KC522070.1 GenBank Accession No: KC522069.1 GenBank Accession No: KC522068.1 GenBank Accession No: KC522067.1, GenBank Accession No: KC522066.1 GenBank Accession No: KC522065.1 GenBank Accession No: KC522064.1, GenBank Accession No: KC522063.1, or GenBank Accession No: KC522062.1, as well as any other subgroup 2b coronavirus now known (e.g., as can be found in the GenBank® Database) or later identified, and any combination thereof.

Nonlimiting examples of a subgroup 2d coronavirus include BtCoV.HKU9.2 (GenBank Accession No. EF065514), BtCoV.HKU9.1 (GenBank Accession No. NC_009021), BtCoV.HkU9.3 (GenBank Accession No. EF065515), BtCoV.HKU9.4 (GenBank Accession No. EF065516), as well as any other subgroup 2d coronavirus now known (e.g., as can be found in the GenBank® Database) or later identified, and any combination thereof.

Nonlimiting examples of a subgroup 3 coronavirus include IBV.Beaudette.IBV.p65 (GenBank Accession No. DQ001339), as well as any other subgroup 3 coronavirus now known (e.g., as can be found in the GenBank® Database) or later identified, and any combination thereof.

A “subject” can also be any animal that is susceptible to infection by coronavirus and/or susceptible to diseases or disorders caused by coronavirus infection. A subject of this invention can be a mammal and in particular embodiments is a human, which can be an infant, a child, an adult or an elderly adult. A “subject at risk of infection by a coronavirus” or a “subject at risk of coronavirus infection” is any subject who may be or has been exposed to a coronavirus. The subject may be a primary contact of an individual diagnosed with a coronavirus infection. “Subject” includes any human or non-human animal. Thus, in addition to being useful for human treatment, the compounds of the present invention may also be useful for veterinary treatment of mammals, including companion animals and farm animals, such as, but not limited to dogs, cats, horses, cows, sheep, and pigs.

A subject in need thereof may be an individual who is displaying a symptom of a coronavirus infection or who has been diagnosed with a coronavirus infection. Further, a subject in need thereof may be one who has been clinically or biochemically determined to be infected with a coronavirus. In one embodiment, the subject may be asymptomatic.

As discussed above, the inventors have identified compounds for the treatment and/or prevention of respiratory diseases or conditions, particularly those associated with coronavirus. Specifically, the compounds significantly reduce coronavirus viral load in the upper respiratory tract, particularly the nose and throat. Surprisingly, the compounds significantly reduce viral load in an animal model challenged with a coronavirus from the genera Alphacoronavirus, specifically the subgroup 2b, more specifically SARS-CoV2. The compound may be any compound of the invention as described herein.

At least preferred embodiments of the compound of the invention are TLR2 agonists.

Toll-Like Receptors (TLRs)

Toll-Like Receptors (TLRs) are pattern recognition receptors (PRRs) expressed by diverse cell types that play an important role in both innate and adaptive immunity. Cells of the innate immune system respond to TLR activation by producing pro-inflammatory cytokines and chemokines that signal for the clearance of the pathogens and damaged-self. Upon engagement with specific ligands, TLR activation leads to the activation of transcription factors such as nuclear factor kappa B (NF)-kB, activating protein-1 (AP-1) and interferon regulatory factors (IRFs) through several adaptor molecules including myeloid differentiation primary response gene 88 (MyD88), Toll-interleukin 1 receptor (TIR) domain containing adaptor protein TIRAP and TIR-domain containing adaptor inducing interferon-beta TRIF, to regulate cytokine expression.

There are a number of TLRs that belong to this membrane receptor protein family including TLR1, TLR3, TLR4, TLR5, TLR6, TLR7, TLR8 and TLR9.

As used herein, the term “TLR2” is intended to mean Toll-Like Receptor 2 protein. In humans, TLR2 is encoded by the TLR2 gene. TLR2 is expressed on the surface of certain cells and plays a fundamental role in pathogen recognition and activation of innate immunity.

A TLR2 agonist is an agent that binds Toll-like receptor 2. The TLR2 agonist may bind to, and activate, TLR2 as a homodimer or heterodimer. Any TLR2 agonist known in the art is contemplated for use in the invention. Preferably, the TLR2 agonist is an agonist of a heterodimer of TLR2 and TLR6.

In any aspect, the TLR2 agonist is an agonist of a heterodimer of TLR2 and TLR6 but not a heterodimer of TLR2 and TLR1.

In any aspect, the TLR2 agonist has a greater affinity for or capacity to activate a heterodimer of TLR2 and TLR6 than a heterodimer of TLR2 and TLR1.

In any aspect, the TLR2 agonist is an agonist of a heterodimer of TLR2 and TLR6 only.

In any embodiment of the invention, the TLR2 agonist comprises a lipid, a peptidoglycan, a lipoprotein or a lipopolysaccharide. Preferably, the TLR2 agonist comprises palmitoyl, myristoyl, stearoyl, lauroyl, octanoyl, or decanoyl. The TLR2 agonist may be selected from the group consisting of: Pam2Cys, Pam3Cys, Ste2Cys, Lau2Cys, and Oct2Cys. In a preferred embodiment, the TLR2 agonist comprises Pam2Cys.

An exemplary lipopeptide in accordance with any embodiment of the present invention is the lipopeptide “Pam2Cys”. One of skill in the art would understand that the term “lipopeptide” means any composition of matter comprising one or more lipid moieties and one or more amino acid sequences that are conjugated. “Pam2Cys” (also known as dipalmitoyl-S-glyceryl-cysteine or S-[2, 3 bis(palmitoyloxy) propyl] cysteine has been synthesised and corresponds to the lipid moiety of MALP-2, a macrophage-activating lipopeptide isolated from Mycoplasma fermentans. Pam2Cys is known to be a ligand of TLR2.

Pam2Cys has the structure:

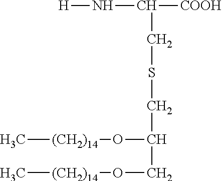

As used herein, reference to “S” as denoted in the above chemical structure defines a sulfur atom.

Another exemplary lipopeptide is the lipoamino acid N-palmitoyl-S-[2, 3-bis (palmitoyloxy) propyl] cysteine, also known as Pam3Cys or Pam3Cys-OH is a synthetic version of the N-terminal moiety of Braun's lipoprotein that spans the inner and outer membranes of Gram negative bacteria Pam3Cys has the following structure:

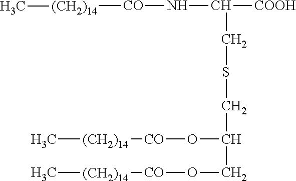

U.S. Pat. No. 5,700,910 describes several N-acyl-S-(2-hydroxyalkyl) cysteines for use as intermediates in the preparation of lipopeptides that are used as synthetic adjuvants, B lymphocyte stimulants, macrophage stimulants, or synthetic vaccines. U.S. Pat. No. 5,700,910 also teaches the use of such compounds as intermediates in the synthesis of Pam3Cys-OH and of lipopeptides that comprise this lipoamino acid or an analog thereof at the N-terminus.

Other lipid moieites which may be used to target cell surface TLRs include palmitoyl, myristoyl, stearoyl, lauroyl, octanoyl, or decanoyl.

In addition to Pam2Cys and Pam3Cys, the present invention also contemplates the use of Ste2Cys, Lau2Cys and Oct2Cys according to the present invention. Those skilled in the art will be aware that Ste2Cys is also known as S-[2, 3-bis (stearoyloxy) propyl] cysteine or distearoyl-S-glyceryl-cysteine; that Lau2Cys is also known as S-[2, 3-bis (lauroyloxy) propyl] cysteine or dilauroyl-S-glyceryl-cysteine); and that Oct2Cys is also known as S-[2,3-bis (octanoyloxy) propyl] cysteine or dioctanoyl-S-glyceryl-cysteine).

Other suitable TLR2 agonists include, but are not limited to, synthetic triacylated and diacylated lipopeptides, FSL-1 (a synthetic lipoprotein derived from Mycoplasma salivarium 1), Pam3Cys (tripalmitoyl-S-glyceryl cysteine) and S-[2,3-bis(palmitoyloxy)-(2RS)-propyl]-N-palmitoyl-(R)-cysteine, where “Pam3” is “tripalmitoyl-S-glyceryl”. Derivatives of Pam3Cys are also suitable TLR2 agonists, where derivatives include, but are not limited to: S-[2,3-bis(palmitoyloxy)-(2-R,S)-propyl]-N-palmitoyl-(R)-Cys-(S)-Ser-(Lys)4-hydroxytrihydrochloride; Pam3Cys-Ser-Ser-Asn-Ala; Pam3Cys-Ser-(Lys)4; Pam3Cys-Ala-Gly; Pam3Cys-Ser-Gly; Pam3Cys-Ser; Pam3Cys-OMe; Pam3Cys-OH; PamCAG, palmitoyl-Cys((RS)-2,3-di(palmitoyloxy)-propyl)-Ala-Gly-OH, and the like.

Other non-limiting examples of suitable TLR2 agonists are Pam2CSK4 Pam2CysSK4 (dipalmitoyl-S-glyceryl cysteine-serine-(lysine)4; or Pam2Cys-Ser-(Lys)4) is a synthetic diacylated lipopeptide. Other synthetic TLRs agonists include those described, e.g., in Kellner et al. (1992) Biol. Chem. 373:1:51-5; Seifer et al. (1990) Biochem. J, 26:795-802; and Lee et al. (2003) J. Lipid Res., 44:479-486.

A TLR2 agonist may be conjugated with one or more compounds or functional groups. Examples of particular compounds or functional groups are given below. One form of compound or functional group may act to increase the solubility of the TLR2 agonist. As will be understood by persons skilled in the art, TLR2 agonists are typically non-polar and, accordingly, while being soluble in non-polar solvents, are only less soluble in polar and aqueous solvents. Where it is desired to use the TLR2 agonist in a polar or aqueous solvent, the TLR2 agonist may be conjugated with a solubilising agent.

A solubilising agent may include one, or more than one, solubilising agent which may be conjugated to TLR2 agonist in order to improve the solubility of the TLR2 moiety. The solubilising agent will generally be a polar moiety which increases the solubility of the TLR2 moiety in polar or aqueous solvents.

In any aspect of the invention, the solubilising agent may be a positively charged group. Positively charged groups of the present invention include but are not limited to penetratin, HIV Tat 48-60, HIV Rev 34-50, transportan, oligoarginine peptides (linear and branched), oligolysine peptides, pyrrrochoricin, alpha-helical amphipathic model peptide, polylysine, protamine, FL17, Magnafloc 1697, and the polycationic compounds described in U.S. Pat. Nos. 6,689,478 and 4,035,558.

In yet a further embodiment of the present invention, the solubilising agent comprises, consists essentially of, or consists of a linear or branched peptide. Typically, the linear or branched peptide contains positively or negatively charged amino acids. Positively charged amino acids may be lysine, arginine, histidine, ornithine or combinations thereof. The branched or linear peptide may contain at least one lysine or arginine residue. Preferably, the charged amino acids are terminal, for example N-terminal. The branched peptides may have one of the following structures.

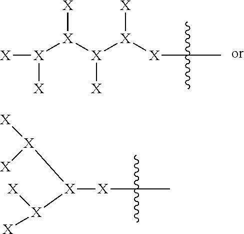

In the above structures X may independently be a charged residue, either a positively or negatively charged residue. Preferably the positively charged amino acids are lysine, arginine, histidine or ornithine. Preferably, the negatively charged amino acids are glutamate or aspartate.

As used herein, ‘PEG’ refers to the polymer compound polyethylene glycol. Unless otherwise defined, reference to ‘PEG’ includes any length polymer of ethylene oxide. Reference to PEG also includes substituted PEG.

The compound or functional group which can act as a solubilising agent may be one or more of the group consisting of “PEG” (or polyethyleneglycol) and a polar polypeptide such as “R4”, a hyper-branched tetra arginine complex; “H4”, a hyper-branched tetra histidine complex; “H8”, a linear peptide containing histidine residues; and ‘E8” a linear peptide containing glutamate residues. Other linear and branched lipid solubilising agents are also envisaged, including a hyper-branched peptide containing glutamate residues (see, e.g., “branched E8”, below). In yet a further embodiment of the present invention, the solubilising agent includes PEG and one or more of the group consisting of R4, H4, H8 and E8 (linear or branched). R4, H4, H8 and E8 have been previously described in PCT/AU2009/000469 (WO/2010/115230) and have the following structures:

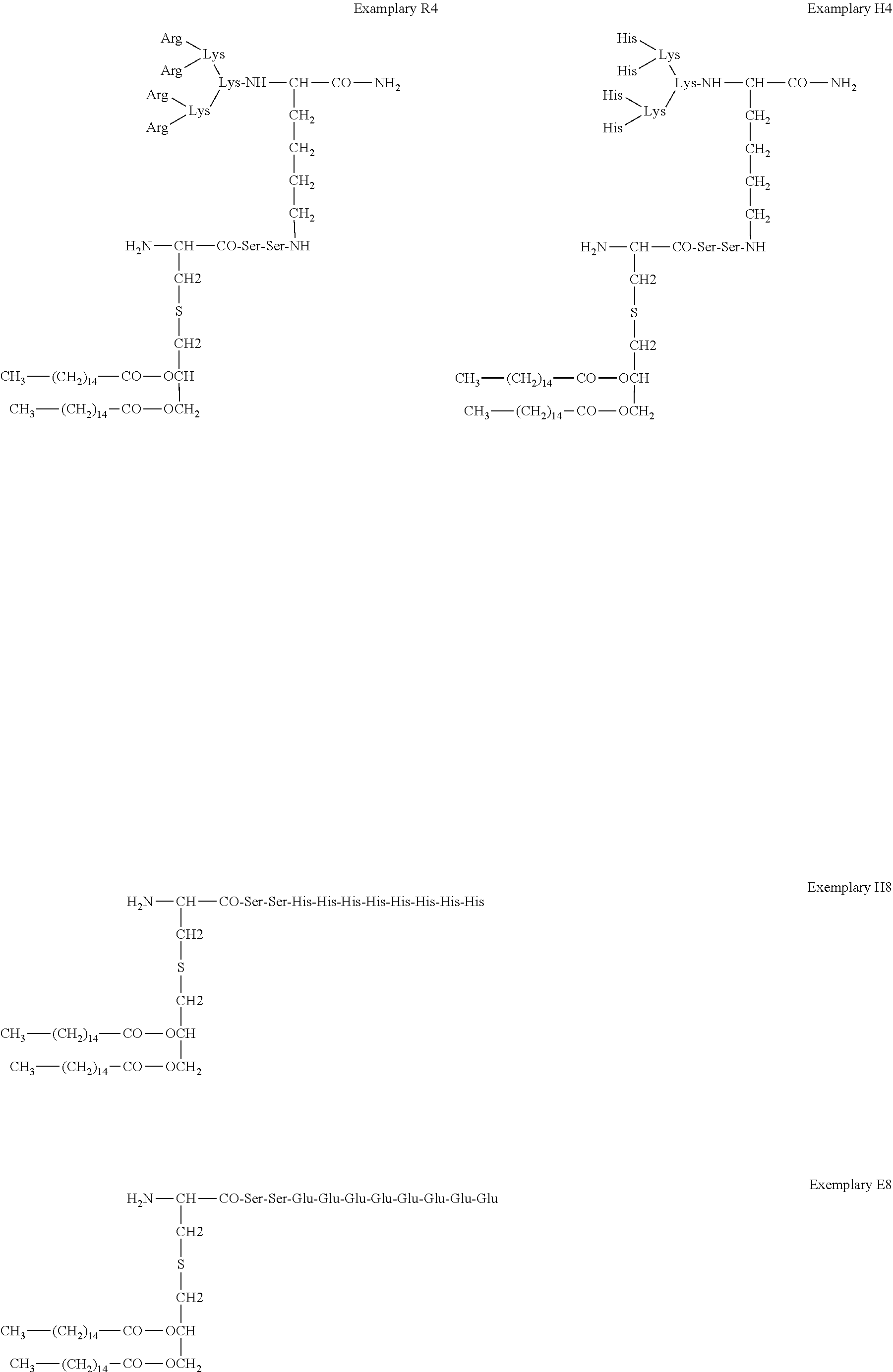

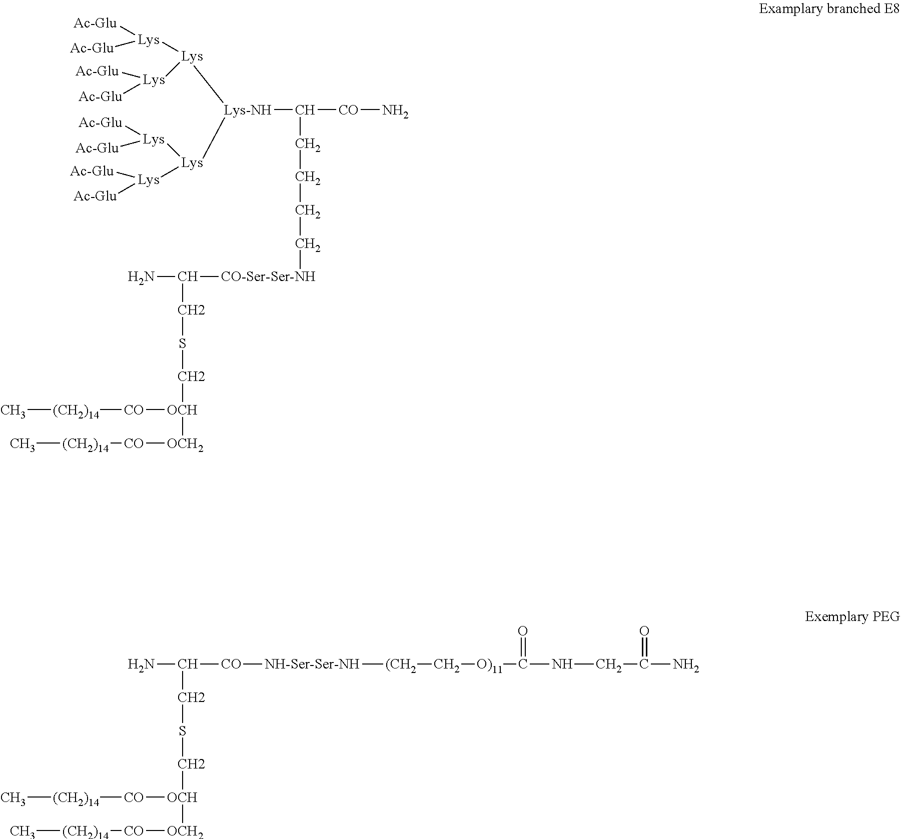

Following are schematic representations of some examples of branched (structures 1-5) and linear (structures 6-8) immunogenic compositions comprising of positively charged (Arginine, R; Lysine, K) or negatively charged (Aspartic acid, D; Glutamic acid, E) amino acids in terminal positions such that their respective electrostatic charges are displayed to the environment. Each immunogenic composition also contains dipalmitoyl-S-glyceryl cysteine (Pam2Cys) which is a ligand for Toll-Like Receptor 2. Two serine residues (Ser) are also incorporated. In the case of construct 2 the peptide structure was assembled in the direction N→C, all other structures may be assembled C→N. Positive and negative electrostatic charges are shown as 2−, 2+, 1−, 1+ etc. depending on the size of charge. Ac=acetyl group used to suppress the positive charge of alpha amino groups in the case of N-terminally situated Glutamic acid.

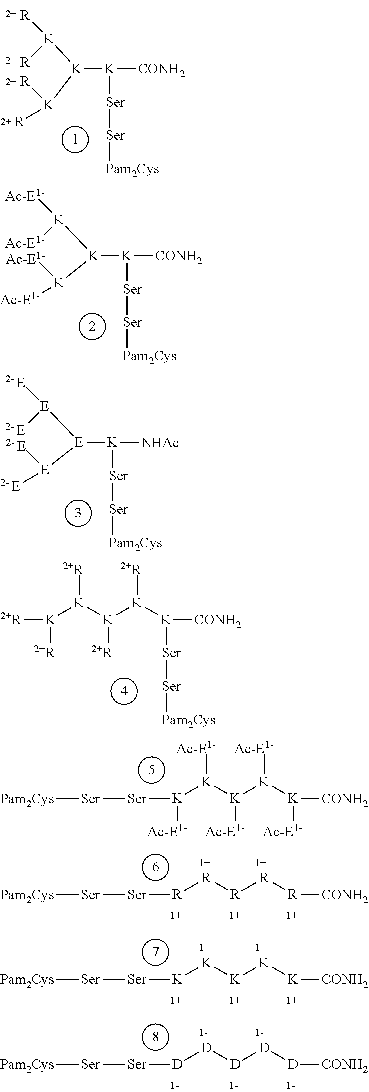

A person skilled in the art will appreciate that the present invention is not limited to the particular exemplified compounds or functional groups that can act as solubilising agents, and that other suitable compounds or functional groups including those that can act as solubilising agents known in the art may be used in accordance with the present invention, such as carbohydrates.

The way in which the one or more compounds or functional group (such as solubilising agents) may be conjugated to a lipid according to the present invention would be well known to a person skilled in the art. For example, conjugation via Fmoc chemistry, through a disulfide or a thioether bridge, or via oxime chemistry is envisaged. In a particular embodiment of the present invention, a soluble form of Pam2Cys was prepared by addition of O—(N-Fmoc-2-aminoethyl)-O′-(2-carboxyethyl)-undecaethyleneglycol (Fmoc-PEOn-OH, Merck Ltd) to Pam2Cys. This resulted in the formation of a PEGylated form of the lipid, Pam2Cys-PEG₁₁ which is then suitable for administration to a subject.

In another form of the invention, the TLR2 moiety comprises a conjugate comprising Pam2Cys conjugated to a pendant R4 form. In a preferred form, pendant-Pam2Cys is conjugated to R4 according to the following structure:

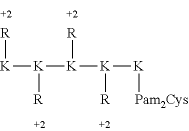

In a preferred form according to any embodiment of the present invention, the TLR2 moiety comprises a conjugate comprising Pam2Cys conjugated to PEG. In a preferred form according to any embodiment of the present invention, the TLR2 moiety comprises a conjugate comprising Pam2Cys conjugated to PEG₁₁ or PEG₁₂. Preferably, the Pam2Cys and PEG₁₁ or PEG₁₂ molecules are separated by at least two serines (PEG₁₁SS-Pam2Cys or PEG₁₂-SS-Pam2Cys).

As used herein, reference to a TLR2 agonist also includes a pharmaceutically acceptable salt, solvate, polymorph or prodrug thereof.

Additional compounds that comprise a TLR2 agonist that are useful in any aspect of the present invention are described below.

In any aspect, the compound may be a compound of formula (I):

A-Y—B (1)

wherein A comprises or consists of a moiety selected from A1 and A2:

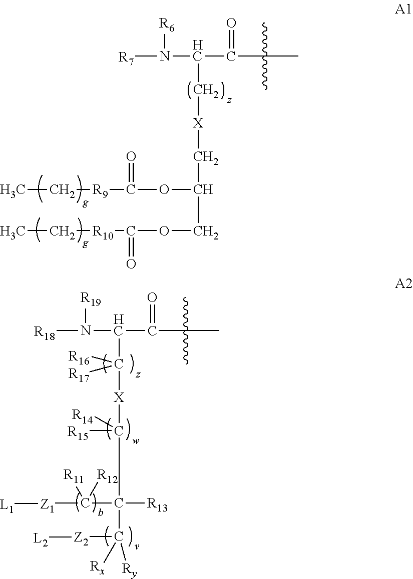

wherein

each z is independently selected from 1 or 2;

each X is independently selected from —S—, —S(═O)— and —S(═O)₂—;

in moiety A1:
each g is independently 10, 11, 12, 13, 14, 15, 16, 17 or 18;
R₆ and R₇ are independently selected from the group consisting of H, a straight or branched C₁-C₄ alkyl, and —C(═O)CH₃;
R₉ and R₁₀ are independently selected from the group consisting of —NH—, —O— or a single bond; and

in moiety A2:
b and w are each independently an integer from 0 to 7 and v is an integer from 0 to 5, such as from 2 to 5, provided that:
   the sum of b, v, and w is at least 3; and
   the sum of b and w is from 0 to 7;
Z₁ and Z₂ are each independently selected from the group consisting of —O—, —NR—,
—S—, S(═O), —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A₂;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;

Y is

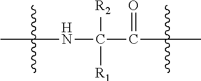

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

and

B comprises or consists of Polyethylene Glycol (PEG),

or a pharmaceutically acceptable salt, solvate or prodrug thereof.

In any aspect, the compound may be a compound of formula (IA1):

A-Y—B (IA1)

wherein A comprises or consists of moiety A1:

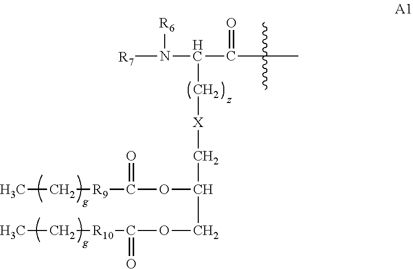

wherein each g is independently 10, 11, 12, 13, 14, 15, 16, 17 or 18;

z is 1 or 2;

X is selected from —S—, —S(═O)— and —S(═O)₂—;

R₆ and R₇ are independently selected from the group consisting of H, a straight or branched C₁-C₄ alkyl, and —C(═O)CH₃;

R₉ and R₁₀ are independently selected from the group consisting of —NH—, —O— or a single bond;

Y is

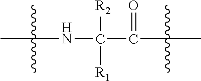

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

and

B comprises or consists of Polyethylene Glycol (PEG),

or a pharmaceutically acceptable salt, solvate or prodrug thereof.

In some embodiments, g is an integer from 12 to 16.

In some embodiments, g is 14.

In any aspect, the compound may be a compound of formula (IA2):

A-Y—B (IA2)

wherein A comprises or consists of:

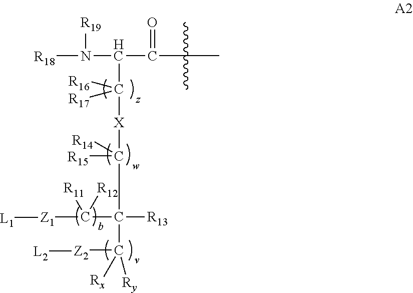

wherein

b and w are each independently an integer from 0 to 7 and v is an integer from 0 to 5, such as from 2 to 5, provided that:
the sum of b, v, and w is at least 3; and
the sum of b and w is from 0 to 7;

z is 1 or 2;

X is selected from —S—, —S(═O)— and —S(═O)₂—;

Z₁ and Z₂ are each independently selected from the group consisting of —O—, —NR—, —S—, S(═O), —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;

R₁₁, R₁₂, Rₓ, R_{y}, R₁₄, R₁₅, R₁₆, and R₁₇ are each independently H or C₁-C₆ aliphatic;

R, R₁₃ and R₁₈ are each independently H or C₁-C₆ aliphatic;

R₁₉ is H, C₁-C₆ aliphatic, an amino protecting group, L₃-C(═O)—, or A₂;

L₁ and L₂ are each independently C₅-C₂₁ aliphatic or C₄-C₂₀ heteroaliphatic;

L₃ is C₁-C₂₁ aliphatic or C₂-C₂₀ heteroaliphatic;

A₂ is an amino acid or a peptide;

wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;

Y is

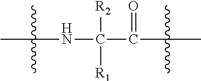

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

and

B comprises or consists of Polyethylene Glycol (PEG),

or a pharmaceutically acceptable salt, solvate or prodrug thereof.

In some embodiments, v is an integer selected from 2, 3, 4 or 5. In some embodiments, v is 2 or 3. In some embodiments, v is 2.

In some embodiments, Rₓ, R_{y}, R₁₁, R₁₂, R₁₃, R₁₄, R₁₅, R₁₆, and R₁₇ are H.

In some embodiments, R and R₁₃ are each H.

In some embodiments, Z₁ and Z₂ are the same and selected from the group consisting of —O—, —NR—, —S—, S(═O), S(═O)₂—, —C(═O)O—, —OC(═O)—, —C(═O)NR—, —NRC(═O)—, —C(═O)S—, —SC(═O)—, OC(═O)O—, NRC(═O)O—, —OC(═O)NR—, and —NRC(═O)NR—.

In some embodiments, Z₁ and Z₂ are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—.

In some embodiments, w is an integer selected from 1-7. In some embodiments, w is 1.

In some embodiments, b is 0.

In some embodiments, the sum of b and w is from 1 to 7. In these embodiments, b may be an integer selected from 0-7 and w may be an integer selected from 1-7, preferably 1.

In some embodiments, b is 0, w is 1 and v is 2.

In some embodiments, R₁₈ is H.

In some embodiments, R₁₉ is selected from the group consisting of H, C₁-C₆ alkyl, —C(═O) C₁-C₆ alkyl or —C(═O)C₁₁-C₁₉alkyl.

In some embodiments, L₁ and L₂ are independently selected from C₅-C₂₁ aliphatic or C₄-C₂₀ heteroaliphatic. In some embodiments, L₁ and L₂ and independently selected from C₁₀-C₁₈ aliphatic or C₁₀-C₁₈ heteroaliphatic. In some embodiments, L₁ and L₂ are independently selected from C14-alkyl and C15-alkyl.

In some embodiments, X is S.

In some embodiments, X is S(═O).

In some embodiments, X is S(═O)₂.

In some embodiments, the invention provides a compound of formula (I) wherein:

v is an integer selected from 2 to 5;
b is 0;

Rₓ, R_{y}, R₁₃, R₁₄, R₁₅, R₁₆, and R₁₇ are H;

Z₁ and Z₂ are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;

w is an integer selected from 1 to 7;

R₁₉ is selected from the group consisting of H, C₁-C₆ alkyl, —C(═O) C₁-C₆ alkyl or —C(═O)C₁₁-C₁₉alkyl; and

L₁ and L₂ and independently selected from C₁₀-C₁₈ aliphatic or C₁₀-C₁₈ heteroaliphatic.

In some embodiments, the invention provides a compound wherein

v is 2;

b is 0;

w is 1;

the sum of v, b and w is 3;

the sum of b and w is 1;

z is 1;

X is S

Z₁ and Z₂ are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;

R₁₁, R₁₂, Rₓ, R_{y}, R₁₄, R₁₅, R₁₆, and R₁₇ at each instance of b, v, w, and z are each H;

R and R₁₃ are each H;

R₁₈ is H;

R₁₉ is selected from the group consisting of H, C₁-C₆ alkyl, —C(═O) C₁-C₆ alkyl or —C(═O)C₁₁-C₁₉alkyl; and

L₁ and L₂ and independently selected from C₁₀-C₁₈ aliphatic or C₁₀-C₁₈ heteroaliphatic.

It will be appreciated that any embodiment of a substituent described herein, including substituents R₁, R₂, R₄, R₅, R₆, R₇, R₉, R₁₀, z, X, g, R₁₁, R₁₂, R₁₃, R₁₄, R₁₅, R₁₆, R₁₇, R₁₈, R₁₉, Rₓ, R_{y}, L₁, L₂, Z₁, Z₂, b, v, w, n, m, p, q, R₃, L, t, k and h, is intended to apply to any instance of that substituent for any compound described herein, including compounds of formulas (I)-(XIX).

In any aspect, the compound may be a compound of formula (II):

A-Y′—B (II)

wherein A comprises or consists of moiety A1 or A2 as defined herein;

Y′ is

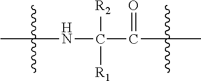

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

and

B comprises or consists of Polyethylene Glycol (PEG),

or a pharmaceutically acceptable salt, solvate or prodrug thereof.

In some embodiments, the compound comprises moiety A1, wherein:

each g is independently 10, 11, 12, 13, 14, 15, 16, 17 or 18;

z is 1;

X is S;

R₆ and R₇ are H;

R₉ and R₁₀ are both a single bond.

In some embodiments, moiety A1 is defined by moiety A1′

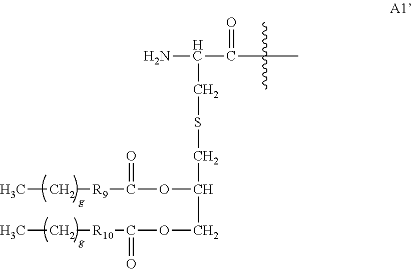

wherein each g is independently 10, 11, 12, 13, 14, 15, 16, 17 or 18.

or a salt, solvate or prodrug thereof.

In any aspect, any of the compounds described herein may be a compound comprising a moiety A selected from A1′ and A2 as defined herein and PEG, wherein the moiety A and PEG are linked by a glycine, serine, homoserine, threonine, phosphoserine, asparagine or glutamine residue, or an ester of a glutamine residue.

In any aspect, the compound may comprise or consist of partial structure A1Y′ or A2Y′:

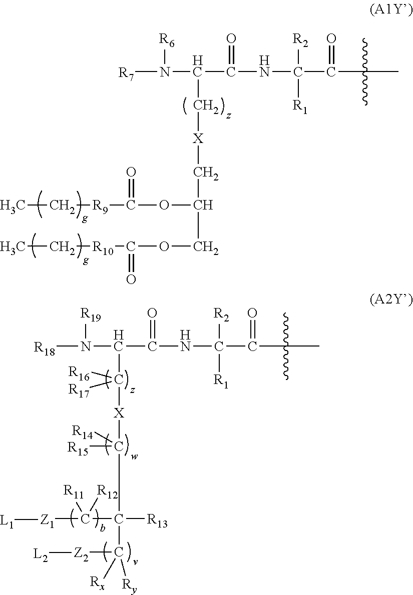

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are independently selected from the group consisting of H, a straight or branched C₁-C₄ alkyl, and —C(═O)CH₃;

R₈ is selected from the group consisting of H and a straight or branched C₁-C₆ alkyl;

R₉ and R₁₀ are independently selected from the group consisting of —NH—, —O— or a single bond;

z is 1 or 2;

X is selected from —S—, —S(═O)— and —S(═O)₂—;

b and w are each independently an integer from 0 to 7 and v is an integer from 0 to 5, provided that:

the sum of b, v, and w is at least 3; and

the sum of b and w is from 0 to 7;

Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;

R₁₁, R₁₂, Rₓ, R_{y}, R₁₄, R₁₅, R₁₆, and R₁₇ at each instance of b, v, w, and z are each independently H or C₁-C₆ aliphatic;

R, R₁₃ and R₁₈ are each independently H or C₁-C₆ aliphatic;

R₁₉ is H, C₁-C₆ aliphatic, an amino protecting group, L₃-C(═O)—, or A₂;

L₁ and L₂ are each independently C₅-C₂₁ aliphatic or C₄-C₂₀ heteroaliphatic;

L₃ is C₁-C₂₁ aliphatic or C₂-C₂₀ heteroaliphatic;

A₂ is an amino acid or a peptide;

wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted; and

A1Y′ or A2Y′ is covalently linked to polyethylene glycol (PEG),

or a pharmaceutically acceptable salt, solvate or prodrug thereof.

In some embodiments, the moiety A and PEG are linked by a serine, homoserine, threonine or phosphoserine residue.

In some embodiments, moiety A and PEG are covalently linked to the glycine, serine, homoserine, threonine, phosphoserine, asparagine or glutamine residue, or an ester of a glutamine residue, through the bond(s) denoted by [symbol] .

In any aspect, the compound may be:

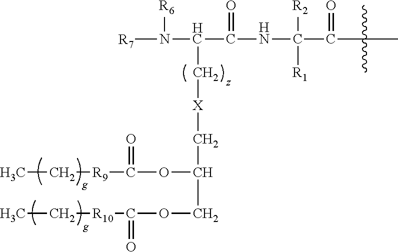

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are independently selected from the group consisting of H, a straight or branched C₁-C₄ alkyl, and —C(═O)CH₃;

R₈ is selected from the group consisting of H and a straight or branched C₁-C₆ alkyl;

R₉ and R₁₀ are independently selected from the group consisting of —NH—, —O— or a single bond;

z is 1 or 2; and

X is selected from —S—, —S(═O)— and —S(═O)₂—;

covalently linked to polyethylene glycol (PEG),

or a pharmaceutically acceptable salt, solvate or prodrug thereof.

In some embodiments, the compound may be:

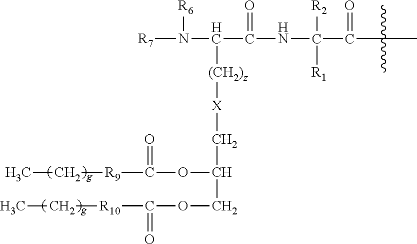

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

R₆ and R₇ are independently selected from the group consisting of H, a straight or branched C₁-C₄ alkyl, and —C(═O)CH₃;

R₉ and R₁₀ are independently selected from the group consisting of —NH—, —O— or a single bond;

z is 1 or 2; and

X is selected from —S—, —S(═O)— and —S(═O)₂—;

covalently linked to polyethylene glycol (PEG),

or a pharmaceutically acceptable salt, solvate or prodrug thereof.

In some embodiments, the compound may be:

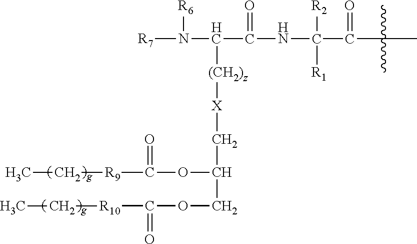

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are H;

R₈ is selected from the group consisting of H and a straight or branched C₁-C₆ alkyl;

R₉ and R₁₀ are both a single bond;

z is 1; and

X is S;

covalently linked to polyethylene glycol (PEG),

or a pharmaceutically acceptable salt, solvate or prodrug thereof.

In some embodiments, the PEG is covalently linked through the bond denoted by [symbol] .

In some embodiments, the compound may be:

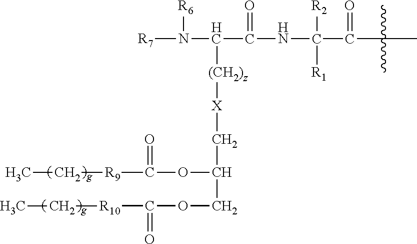

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

R₆ and R₇ are H;

R₉ and R₁₀ are both a single bond;

z is 1; and

X is S;

covalently linked to polyethylene glycol (PEG),

or a pharmaceutically acceptable salt, solvate or prodrug thereof.

In some embodiments, the PEG is covalently linked through the bond denoted by [symbol] .

In some embodiments, the compound may be:

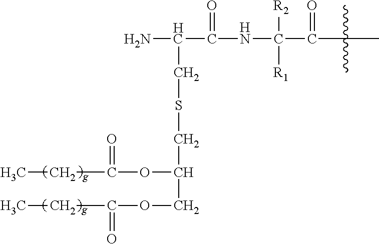

wherein R₁, R₂ and g are as defined herein. salt, solvate or prodrug thereof

In some embodiments, the PEG is covalently linked through the bond denoted by [symbol] .

In some embodiments, the compound may be:

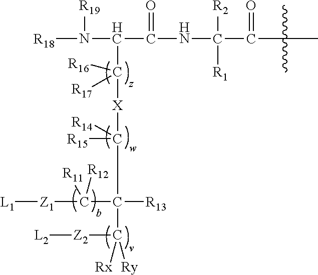

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

b and w are each independently an integer from 0 to 7 and v is an integer from 0 to 5, provided that:

the sum of b, v, and w is at least 3; and

the sum of b and w is from 0 to 7;

z is 1 or 2;

X is selected from —S—, —S(═O)— and —S(═O)₂—;

Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;

R₁₁, R₁₂, Rₓ, R_{y}, R₁₄, R₁₅, R₁₆, and R₁₇ at each instance of b, v, w, and z are each independently H or C₁-C₆ aliphatic;

R, R₁₃ and R₁₈ are each independently H or C₁-C₆ aliphatic;

R₁₉ is H, C₁-C₆ aliphatic, an amino protecting group, L₃-C(═O)—, or A₂;

L₁ and L₂ are each independently C₅-C₂₁ aliphatic or C₄-C₂₀ heteroaliphatic;

L₃ is C₁-C₂₁ aliphatic or C₂-C₂₀ heteroaliphatic;

A₂ is an amino acid or a peptide;

wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;

covalently linked to polyethylene glycol (PEG),

or a pharmaceutically acceptable salt, solvate or prodrug thereof.

In some embodiments, the PEG is covalently linked through the bond denoted by [symbol] .

In any aspect, the compound may be a compound of formula (III):

AY-B (III)

wherein

AY comprises or consists of a moiety selected from AY1 and AY2

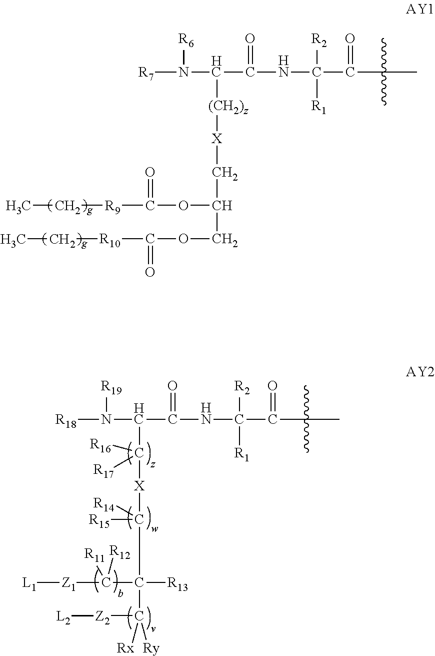

wherein each of R₁, R₂, R₆, R₇, R₉, R₁₀, z, X, g, R₁₁, R₁₂, R₁₃, R₁₄, R₁₅, R₁₆, R₁₇, R₁₈, R₁₉, Rₓ, R_{y}, L₁, L₂, Z₁, Z₂, b, v and w are as defined for the compound of formula (I); and

B comprises or consists of Polyethylene Glycol (PEG).

In any aspect, the compound may be a compound of formula (IV):

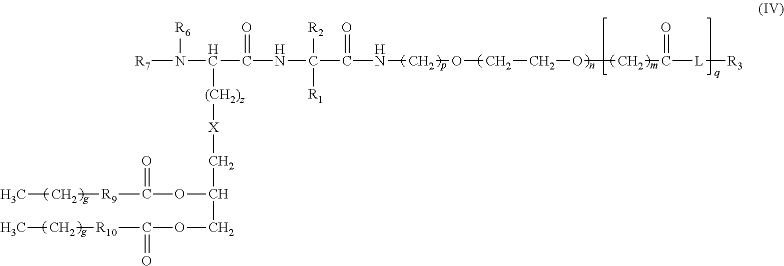

wherein

n is 3 to 100;

m is 1, 2, 3 or 4;

each g is independently 10, 11, 12, 13, 14, 15, 16, 17 or 18;

p is 2, 3 or 4;

q is null or 1;

R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are independently selected from the group consisting of H, a straight or branched C₁-C₄ alkyl, and —C(═O)CH₃;

R₈ is selected from the group consisting of H and a straight or branched C₁-C₆ alkyl;

R₉ and R₁₀ are independently selected from the group consisting of —NH—, —O— or a single bond;

z is 1 or 2;

X is selected from —S—, —S(═O)— and —S(═O)₂—;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

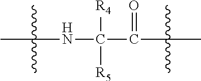

wherein R₄ is H; and

R₅ is the side chain, or second hydrogen of the amino acid

or a pharmaceutically acceptable salt, solvate or prodrug thereof.

In any aspect, the compound may be a compound of formula (V):

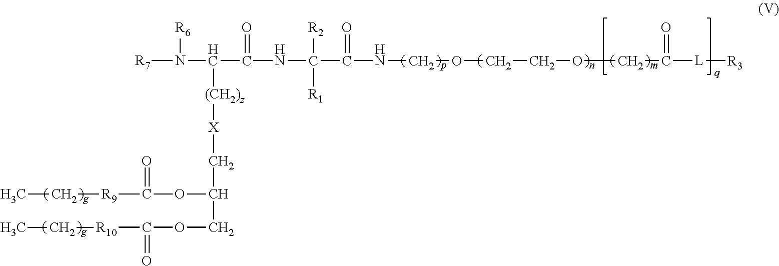

wherein

n is 3 to 100;

m is 1, 2, 3 or 4;

each g is independently 10, 11, 12, 13, 14, 15, 16, 17 or 18;

p is 2, 3 or 4;

q is null or 1;

R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

R₆ and R₇ are independently selected from the group consisting of H, a straight or branched C₁-C₄ alkyl, and —C(═O)CH₃;

R₉ and R₁₀ are independently selected from the group consisting of —NH—, —O— or a single bond;

z is 1 or 2;

X is selected from —S—, —S(═O)— and —S(═O)₂—;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

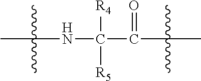

wherein R₄ is H; and

R₅ is the side chain, or second hydrogen of the amino acid

or a pharmaceutically acceptable salt, solvate or prodrug thereof.

In some embodiments, the compound is a compound of formula (IV) or (V) wherein

R₆ and R₇ are H;

R₈ is selected from the group consisting of H and a straight or branched C₁-C₆ alkyl;

R₉ and R₁₀ are both a single bond;

z is 1; and

X is S.

In some embodiments, the compound of any one of formulas (I)-(V) may be a compound of formula (VI):

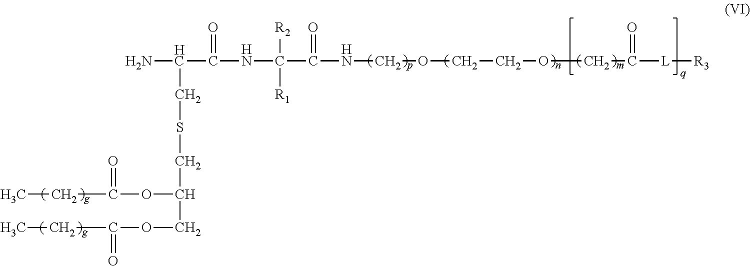

wherein

n is 3 to 100;

m is 1, 2, 3 or 4;

each g is independently 10, 11, 12, 13, 14, 15, 16, 17 or 18;

p is 2, 3 or 4;

q is null or 1;

R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

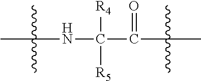

wherein R₄ is H; and

R₅ is the side chain, or second hydrogen of the amino acid

or a pharmaceutically acceptable salt, solvate or prodrug thereof.

In any aspect, the compound may be a compound of formula (VII):

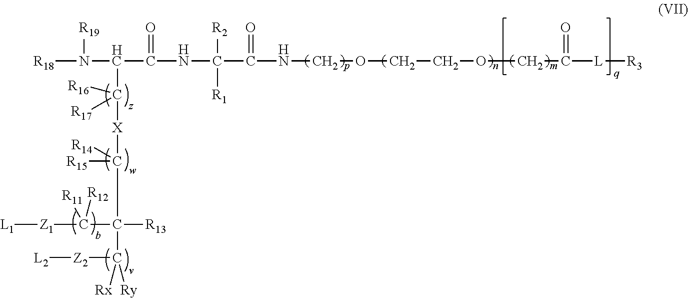

wherein

n is 3 to 100;

m is 1, 2, 3 or 4;

p is 2, 3 or 4;

q is null or 1;

R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and CH₂CH₂C(═O)OR₈, wherein any one of the alkyl hydrogens can be replaced with a halogen;

R₈ is selected from the group consisting of H and a straight or branched C₁-C₆ alkyl;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

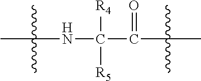

wherein R₄ is H; and

R₅ is the side chain, or second hydrogen of the amino acid;

b and w are each independently an integer from 0 to 7 and v is an integer from 0 to 5, provided that:

the sum of b, v, and w is at least 3; and

the sum of b and w is from 0 to 7;

z is 1 or 2;

X is selected from —S—, —S(═O)— and —S(═O)₂—;

Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;

R₁₁, R₁₂, Rₓ, R_{y}, R₁₄, R₁₅, R₁₆, and R₁₇ at each instance of b, v, w, and z are each independently H or C₁-C₆ aliphatic;

R, R₁₃ and R₁₈ are each independently H or C₁-C₆ aliphatic;

R₁₉ is H, C1-Ce aliphatic, an amino protecting group, L₃-C(═O)—, or A₂;

L₁ and L₂ are each independently C₅-C₂₁ aliphatic or C₄-C₂₀ heteroaliphatic;

L₃ is C₁-C₂₁ aliphatic or C₂-C₂₀ heteroaliphatic;

A₂ is an amino acid or a peptide;

wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;

or a pharmaceutically acceptable salt, solvate or prodrug thereof.

In any aspect, the compound may be a compound of formula (VIII):

A-Y—NH—(CH₂)ₚ—O—(CH₂—CH₂—O)ₙ—[(CH₂)ₘ—CO-L-]_{q}R₃ (VIII)

wherein

A is a moiety selected from A1 and A2 as defined herein

Y is

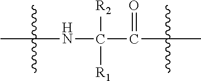

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are independently selected from the group consisting of H, a straight or branched C₁-C₄ alkyl, and —C(═O)CH₃;

R₈ is selected from the group consisting of H and a straight or branched C₁-C₆ alkyl;

R₉ and R₁₀ are independently selected from the group consisting of —NH—, —O— or a single bond;

z is 1 or 2;

X is selected from —S—, —S(═O)— and —S(═O)₂—;

n is 3 to 100;

m is 1, 2, 3 or 4;

each g is independently 10, 11, 12, 13, 14, 15, 16, 17 or 18;

p is 2, 3 or 4;

q is null or 1;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

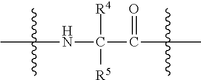

wherein R₄ is H; and

R₅ is the side chain, or second hydrogen of the amino acid,

or a pharmaceutically acceptable salt, solvate or prodrug thereof.

In any aspect, the compound may be a compound of formula (IX):

A1-Y—NH—(CH₂)ₚ—O—(CH₂—CH₂—O)ₙ—[(CH₂)ₘ—CO-L-]_{q}R₃ (IX)

wherein

A1 is represented by moiety A1 as defined for formula (I)

Y is

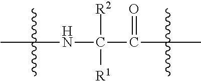

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

R₆ and R₇ are independently selected from the group consisting of H, a straight or branched C₁-C₄ alkyl, and —C(═O)CH₃;

R₉ and R₁₀ are independently selected from the group consisting of —NH—, —O— or a single bond;

z is 1 or 2;

X is selected from —S—, —S(═O)— and —S(═O)₂—;

n is 3 to 100;

m is 1, 2, 3 or 4;

each g is independently 10, 11, 12, 13, 14, 15, 16, 17 or 18;

p is 2, 3 or 4;

q is null or 1;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

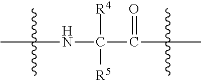

wherein R₄ is H; and

R₅ is the side chain, or second hydrogen of the amino acid,

or a pharmaceutically acceptable salt, solvate or prodrug thereof.

In some embodiments, the compound is a compound of formula (VIII) or (IX), wherein

R₆ and R₇ are H;

R₉ and R₁₀ are both a single bond;

z is 1;

X is S;

salt, solvate or prodrug thereof salt, solvate or prodrug thereof salt, solvate or prodrug thereof In any aspect, the compound may be a compound of formula (X):

Pam2Cys-Y—NH—(CH₂)ₚ—O—(CH₂—CH₂—O)ₙ—[(CH₂)ₘ—CO-L-]_{q}R₃ (X)

wherein

Pam2Cys has the structure:

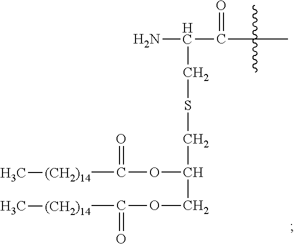

Y is:

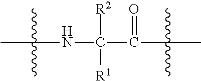

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

n is 3 to 100;

m is 1, 2, 3 or 4;

p is 2, 3 or 4;

q is null or 1;

wherein when q=1, R₃ is H, —NH₂ or —OH;

wherein when q=0, R₃ is H;

L is null or consists of 1 to 10 units, wherein each unit is a natural alpha amino acid or derived from a natural alpha amino acid, and has the formula:

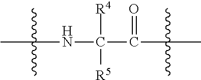

wherein R₄ is H; and

R₅ is the side chain, or second hydrogen of the amino acid,

or a pharmaceutically acceptable salt, solvate or prodrug thereof.

In any aspect, the compound may be a compound of formula (XI):

Pam2Cys-Y—NH—(CH₂)ₚ—O—(CH₂—CH₂—O)ₙ—[(CH₂)ₘ—CO-L-]_{q}R₃ (XI)

wherein

Pam2Cys has the structure:

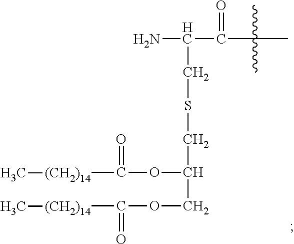

Y is:

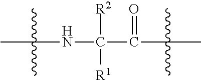

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen and wherein R₁ and R₂ are not both H;

n is 3 to 100;

m is 1, 2, 3 or 4;

p is 2, 3 or 4;

q is null or 1;

wherein when q=1, R₃ is H, —NH₂ or —OH;

wherein when q=0, R₃ is H;

L is null or consists of 1 to 10 units, wherein each unit is a natural alpha amino acid or derived from a natural alpha amino acid, and has the formula:

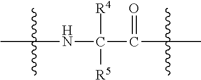

wherein R₄ is H; and

R₅ is the side chain, or second hydrogen of the amino acid,

or a pharmaceutically acceptable salt, solvate or prodrug thereof.

In any aspect, the compound may be a compound of formula (XII):

Pam2Cys-Y—NH—(CH₂)ₚ—O—(CH₂—CH₂—O)ₙ—[(CH₂)ₘ—CO-L-]_{q}R₃ (XII)

wherein

Pam2Cys has the structure:

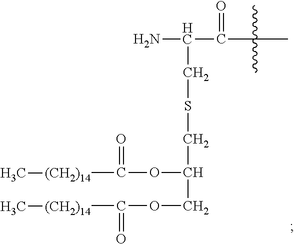

Y is:

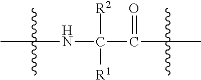

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

n is 3 to 100;

m is 1, 2, 3 or 4;

p is 2, 3 or 4;

q is null or 1;

wherein when q=1, R₃ is H, —NH₂ or —OH;

wherein when q=0, R₃ is H;

L is null or consists of 1 to 10 units, wherein each unit is a natural alpha amino acid or derived from a natural alpha amino acid, and has the formula:

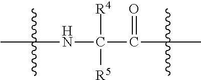

wherein R₄ is H; and

R₅ is the side chain, or second hydrogen of the amino acid,

or a pharmaceutically acceptable salt, solvate or prodrug thereof.

In any aspect, the compound may be a compound of formula (XIII):

Pam2Cys-Ser-NH—(CH₂)ₚ—O—(CH₂—CH₂—O)ₙ—[(CH₂)ₘ—CO-L-]_{q}R₃ (XIII)

wherein

Pam2Cys-Ser has the structure:

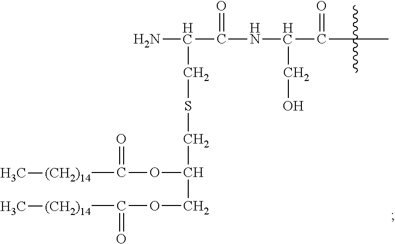

n is 3 to 100;

m is 1, 2, 3 or 4;

p is 2, 3 or 4;

q is null or 1;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

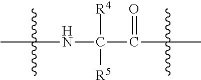

wherein R₄ is H; and

R₅ is the side chain, or second hydrogen of the amino acid,

or a pharmaceutically acceptable salt, solvate or prodrug thereof.

In one embodiment, the compound has the formula (XIV):

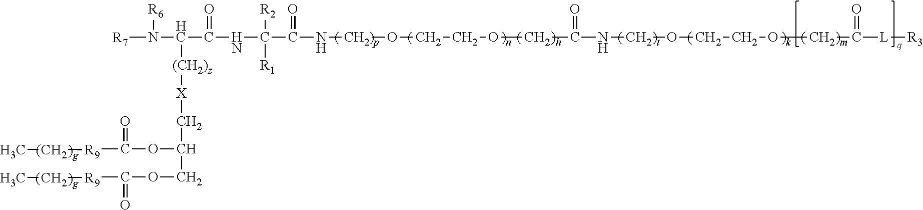

wherein

n is 3 to 100;

k is 3 to 100;

m is 1, 2, 3 or 4;

each g is independently 10, 11, 12, 13, 14, 15, 16, 17 or 18;

p is 2, 3 or 4;

t is 2, 3 or 4;

h is 1, 2, 3 or 4;

q is null or 1;

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are independently selected from the group consisting of H, a straight or branched C₁-C₄ alkyl, and —C(═O)CH₃;

R₈ is selected from the group consisting of H and a straight or branched C₁-C₆ alkyl;

R₉ and R₁₀ are independently selected from the group consisting of —NH—, —O— or a single bond;

z is 1 or 2;

X is selected from —S—, —S(═O)— and —S(═O)₂—;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

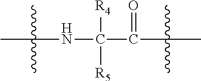

wherein R₄ is H; and

R₅ is the side chain, or second hydrogen of the amino acid,

or a pharmaceutically acceptable salt, solvate or prodrug thereof.

In one embodiment, the compound has the formula (XV):

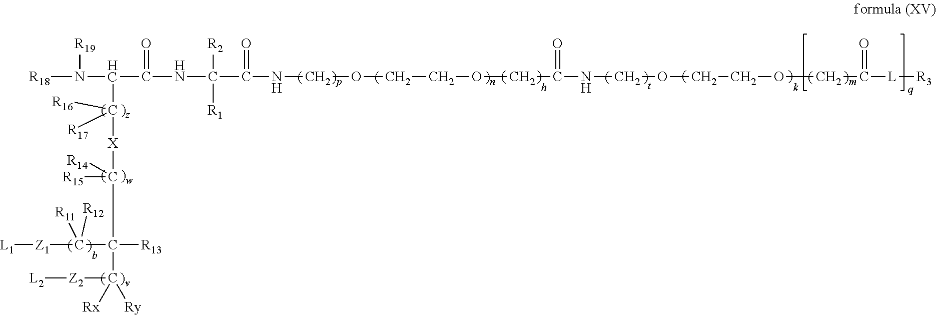

wherein

n is 3 to 100;

k is 3 to 100;

m is 1, 2, 3 or 4;

p is 2, 3 or 4;

t is 2, 3 or 4;

h is 1, 2, 3 or 4;

q is null or 1;

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

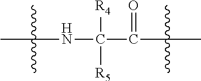

wherein R₄ is H; and

R₅ is the side chain, or second hydrogen of the amino acid;

b and w are each independently an integer from 0 to 7 and v is an integer from 0 to 5, provided that:

the sum of b, v, and w is at least 3; and

the sum of b and w is from 0 to 7;

z is 1 or 2;

X is selected from —S—, —S(═O)— and —S(═O)₂—;

Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;

R₁₁, R₁₂, Rₓ, R_{y}, R₁₄, R₁₅, R₁₆, and R₁₇ at each instance of b, v, w, and z are each independently H or C₁-C₆ aliphatic;

R, R₁₃ and R₁₈ are each independently H or C₁-C₆ aliphatic;

R₁₉ is H, C1-Ce aliphatic, an amino protecting group, L₃-C(═O)—, or A₂;

L₁ and L₂ are each independently C₅-C₂₁ aliphatic or C₄-C₂₀ heteroaliphatic;

L₃ is C₁-C₂₁ aliphatic or C₂-C₂₀ heteroaliphatic;

A₂ is an amino acid or a peptide;

wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;

or a pharmaceutically acceptable salt, solvate or prodrug thereof.

In any aspect, the compound may be a compound of formula (XVI):

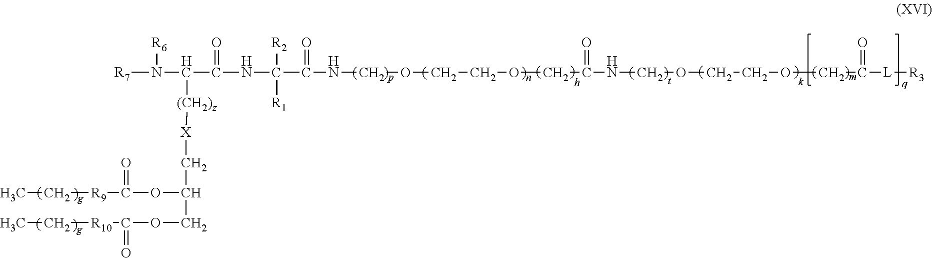

wherein

n is 3 to 100;

k is 3 to 100;

m is 1, 2, 3 or 4;

each g is independently 10, 11, 12, 13, 14, 15, 16, 17 or 18;

p is 2, 3 or 4;

t is 2, 3 or 4;

h is 1, 2, 3 or 4;

q is null or 1;

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

R₆ and R₇ are independently selected from the group consisting of H, a straight or branched C₁-C₄ alkyl, and —C(═O)CH₃;

R₉ and R₁₀ are independently selected from the group consisting of —NH—, —O— or a single bond;

z is 1 or 2;

X is selected from —S—, —S(═O)— and —S(═O)₂—;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

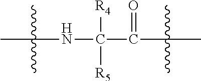

wherein R₄ is H; and

R₅ is the side chain, or second hydrogen of the amino acid,

or a pharmaceutically acceptable salt, solvate or prodrug thereof.

In any aspect, the compound may be a compound of formula (XVII):

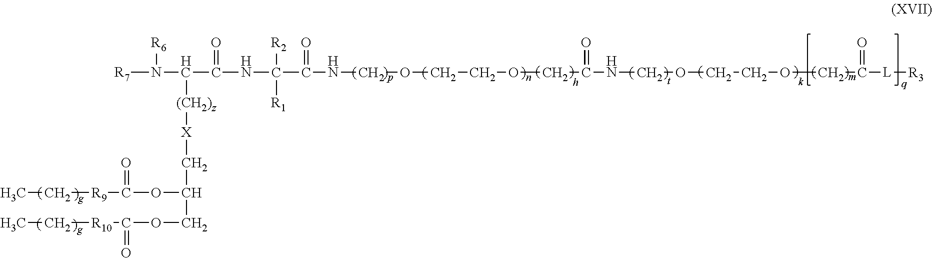

wherein

n is 3 to 100;

k is 3 to 100;

m is 1, 2, 3 or 4;

each g is independently 10, 11, 12, 13, 14, 15, 16, 17 or 18;

p is 2, 3 or 4;

t is 2, 3 or 4;

h is 1, 2, 3 or 4;

q is null or 1;

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are H;

R₈ is selected from the group consisting of H and a straight or branched C₁-C₆ alkyl;

R₉ and R₁₀ are both a single bond;

z is 1;

X is S;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

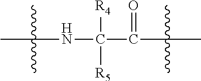

wherein R₄ is H; and

R₅ is the side chain, or second hydrogen of the amino acid,

or a pharmaceutically acceptable salt, solvate or prodrug thereof.

In any aspect, the compound may be a compound of formula (XVIII):

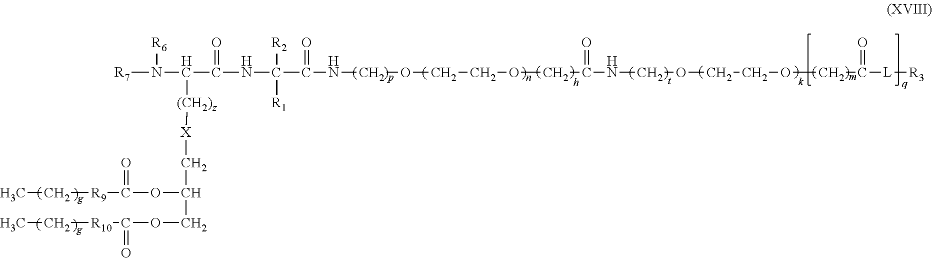

wherein

n is 3 to 100;

k is 3 to 100;

m is 1, 2, 3 or 4;

each g is independently 10, 11, 12, 13, 14, 15, 16, 17 or 18;

p is 2, 3 or 4;

t is 2, 3 or 4;

h is 1, 2, 3 or 4;

q is null or 1;

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

R₆ and R₇ are H;

R₉ and R₁₀ are both a single bond;

z is 1;

X is S;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

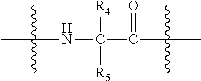

wherein R₄ is H; and

R₅ is the side chain, or second hydrogen of the amino acid,

or a pharmaceutically acceptable salt, solvate or prodrug thereof.

In any aspect, the compound may be a compound of formula (XIX):

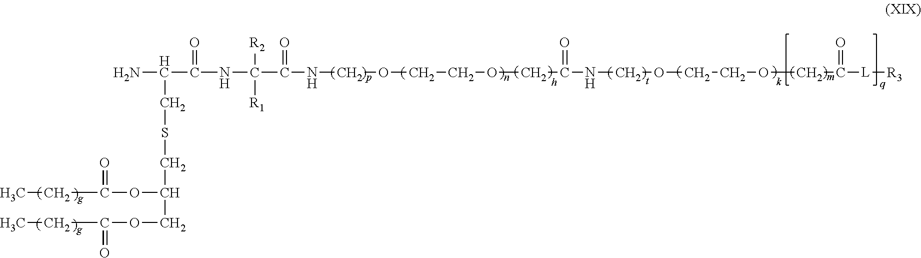

wherein

n is 3 to 100;

k is 3 to 100;

m is 1, 2, 3 or 4;

each g is independently 10, 11, 12, 13, 14, 15, 16, 17 or 18;

p is 2, 3 or 4;

t is 2, 3 or 4;

h is 1, 2, 3 or 4;

q is null or 1;

R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

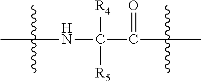

wherein R₄ is H; and

R₅ is the side chain, or second hydrogen of the amino acid,

or a pharmaceutically acceptable salt, solvate or prodrug thereof.

In some embodiments, any compound disclosed herein (including a compound of any one of formulas (I)-(XIX)) that comprises polyethylene glycol (PEG) may comprise the PEG in the form of a substituted PEG.

In some embodiments, the substituted PEG is represented by partial formula B-I:

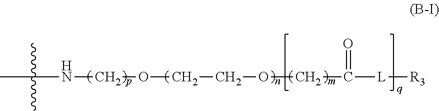

wherein

n is 3 to 100;

m is 1, 2, 3 or 4;

p is 2, 3 or 4;

q is null or 1;

R₃ is H, —NH₂ or —OH, wherein when q is null, R₃ is H and when q is 1, R₃ is —NH₂ or —OH;

L is null or consists of 1 to 10 units, wherein each unit is a natural alpha amino acid or derived from a natural alpha amino acid, and has the formula:

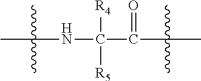

wherein R₄ is H; and

R₅ is the side chain, or second hydrogen of the amino acid.

In some embodiments, the substituted PEG is represented by partial formula B-II:

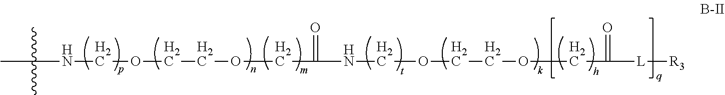

wherein

p is 2, 3 or 4;

n is 3 to 100;

m is 1, 2, 3 or 4;

t is 2, 3 or 4;

k is 3 to 100;

h is 1, 2, 3 or 4;

q is null or 1;

wherein when q is 1, R₃ is —NH₂ or —OH;

wherein when q is null, R₃ is H;

L is null or consists of 1 to 10 units, wherein each unit is a natural alpha amino acid or derived from a natural alpha amino acid, and has the formula:

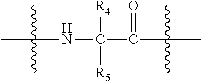

wherein R₄ is H; and

R₅ is the side chain, or second hydrogen of the amino acid.

In some embodiments of the substituted PEG of formula B-I or B-II, q is 1.

In some embodiments of the substituted PEG of formula B-I or B-II, n may be from to 14, such as 11, or from 24 to 30, such as 27.

In some embodiments of the substituted PEG of formula B-I or B-II, m is from 1 to 3, such as 2.

In some embodiments of the substituted PEG of formula B-I or B-II, when q is 1, R₃ is —NH₂.

In some embodiments of the substituted PEG of formula B-I or B-II, L is a natural alpha amino acid residue.

Compounds described herein may exist in and be isolated in optically active and racemic forms. As would be understood by a person skilled in the art, the present invention is intended to encompass any racemic, optically active or stereoisomeric form, or mixtures thereof, of compounds of the invention which possess the useful properties described herein. It is well known in the art how to prepare such forms (for example, by resolution of racemic mixtures by recrystallization, by synthesis from optically-active starting materials, by chiral synthesis, or by chiral chromatographic separation). In some embodiments, a composition may comprise a compound in an enantiomerically or diastereomerically enriched form. For example, the compound may have an enantiomeric excess (ee) or a diastereomeric excess (de) of at least about 1%, 5%, 10%, 20%, 30%, 40%, 50%, 60%, 70%, 80%, 90%, 95%, 96%, 97%, 98%, 99% or more than 99%. In some embodiments, the compound may be enriched by at least about 1%, 5%, 10%, 20%, 30%, 40%, 50%, 60%, 70%, 80%, 90%, 95%, 96%, 97%, 98%, 99% or more than 99% at any stereocentre of the compound.

In any aspect, the compound may comprise a chiral centre around the following chiral centre (shown at *):

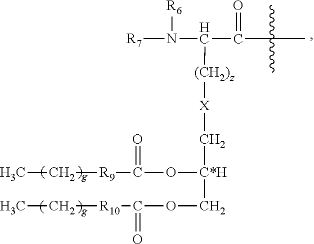

wherein the chiral centre is in the R configuration. A compound in this form may also be referred to as an R-Pam2 analogue diastereomer of a compound of the invention as described herein. This may be depicted as:

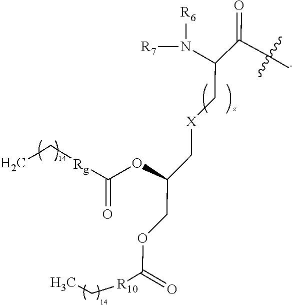

In any aspect, the compound may comprise a chiral centre in the 2,3-bis(palmitoyloxy)propyl moiety of Pam2Cys (shown at *):

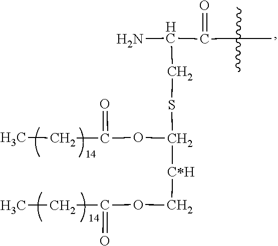

wherein the chiral centre is in the R configuration. A compound in this form may also be referred to as an R-Pam2 diastereomer of a compound of the invention as described herein. This may be depicted as:

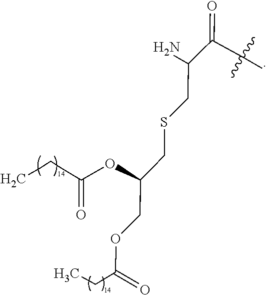

In any aspect, the compound may comprise a chiral centre around the following chiral centre (shown at *):

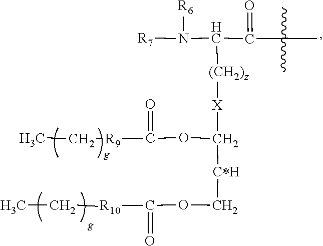

wherein the chiral centre is in the S configuration. A compound in this form may also be referred to as an S-Pam2 analogue diastereomer of a compound of the invention as described herein. This may be depicted as:

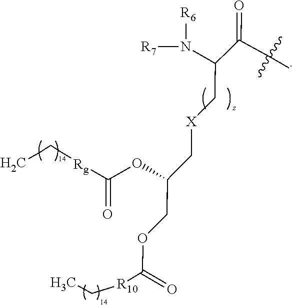

In any aspect, the compound comprises a chiral centre in the 2,3-bis(palmitoyloxy)propyl moiety of Pam2Cys (shown at *):

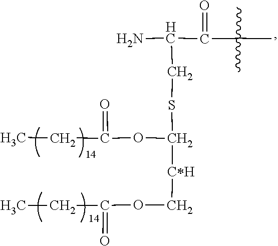

wherein the chiral centre is in the S configuration. A compound in this form may also be referred to as an S-Pam2 diastereomer of a compound of the invention as described herein. This may be depicted as:

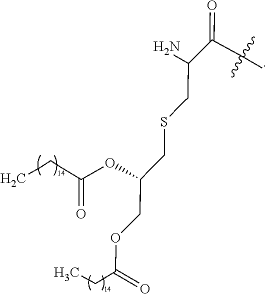

In any aspect, the compound comprises a chiral centre around the following chiral centre (shown at *):

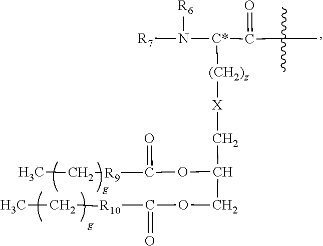

wherein the chiral centre is in the L configuration. A compound in this form may also be referred to as an L-Cys analogue diastereomer of Pam2Cys of a compound of the invention as described herein. This may be depicted as:

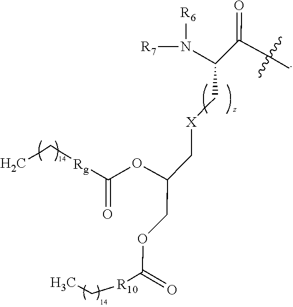

In any aspect, the compound comprises a chiral centre in the cysteine residue of Pam2Cys (shown at *):

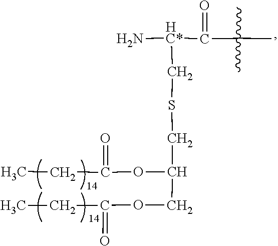

wherein the chiral centre is in the L configuration. A compound in this form may also be referred to as an L-Cys diastereomer of Pam2Cys of a compound of the invention as described herein. This may be depicted as:

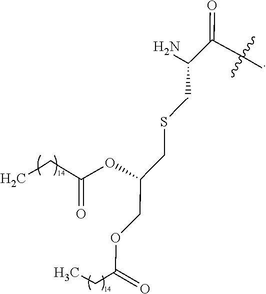

Other stereocentres in these compounds may be racemic or independently enriched in either the R or S configuration.

In any aspect, the compound comprises a chiral centre in moiety A1 around the following chiral centre (shown at *):

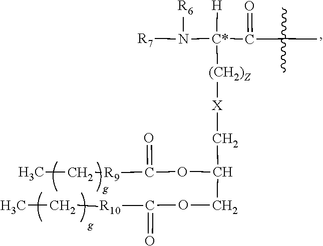

wherein the chiral centre is in the D configuration. A compound in this form may also be referred to as an D-Cys analogue diastereomer of Pam2Cys of a compound described herein. This may be depicted as:

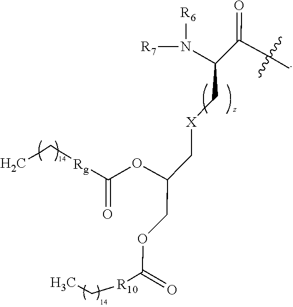

Other stereocentres in these compounds may be racemic or independently enriched in either the R or S configuration.

In any aspect, the compound comprises a chiral centre in the cysteine residue of Pam2Cys (shown at *):

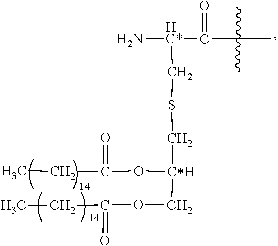

wherein the chiral centre is in the D configuration. A compound in this form may also be referred to as an D-Cys diastereomer of Pam2Cys of a compound described herein. This may be depicted as:

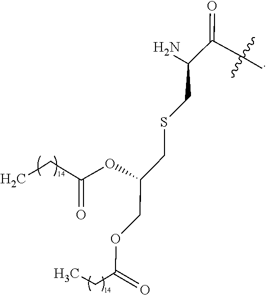

Other stereocentres in these compounds may be racemic or independently enriched in either the R or S configuration.

In any aspect or embodiment of the invention, a compound of the present invention may be provided in a chiral form enriched at a chiral centre at the following carbon atom (shown at *) of moiety A₂:

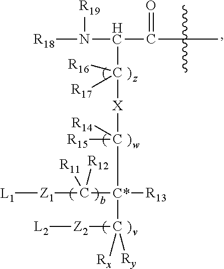

wherein the chiral centre is in the R configuration. In some embodiments, this stereoisomer of the compound may be depicted as:

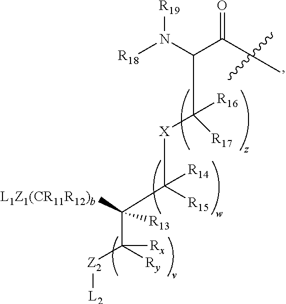

wherein L₁, L₂, Z₁, Z₂, Rₓ, R_{y}, R₁₁, R₁₂, R₁₃, R₁₄, R₁₅, R₁₆, R₁₇, R₁₈, R₁₉, b, v and z are as defined for the compound of Formula (I) and w is 1. Other stereocentres in these compounds may be racemic or independently enriched in either the R or S configuration.

In any aspect or embodiment of the invention, a compound of the present invention may be provided in a chiral form enriched at a chiral centre at the following carbon atom (shown at *) of moiety A2:

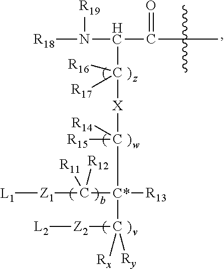

wherein the chiral centre is in the S configuration. In some embodiments, moiety A of this stereoisomer of the compound may be depicted as:

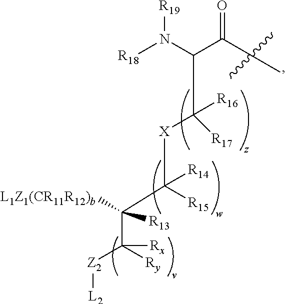

wherein L₁, L₂, Z₁, Z₂, Rₓ, R_{y}, R₁₁, R₁₂, R₁₃, R₁₄, R₁₅, R₁₆, R₁₇, R₁₈, R₁₉, b, v, w, and z are as defined for the compound or Formula (I). Other stereocentres in these compounds may be racemic or independently enriched in either the R or S configuration.

In any aspect or embodiment of the invention, a compound of the present invention may be provided in a chiral form enriched at a chiral centre at the following carbon atom (shown at **) of moiety A2:

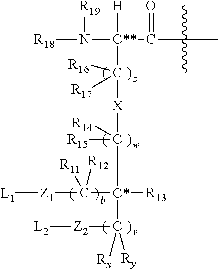

wherein the chiral centre is in the L configuration. A compound in this form may also be referred to as an L-Cys analogue stereoisomer of a compound of the invention. In some embodiments, this stereoisomer of the compound may be depicted as:

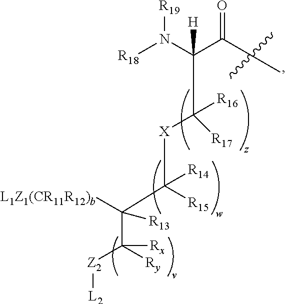

wherein L₁, L₂, Z₁, Z₂, Rₓ, R_{y}, R₁₁, R₁₂, R₁₃, R₁₄, R₁₅, R₁₆, R₁₇, R₁₈, R₁₉, b, v, w, and z are as defined for the compound or Formula (I). Other stereocentres in these compounds may be racemic or independently enriched in either the R or S configuration.

In any aspect or embodiment of the invention, a compound of the present invention may be provided in a chiral form enriched at a chiral centre at the following carbon atom (shown at **) of moiety A2:

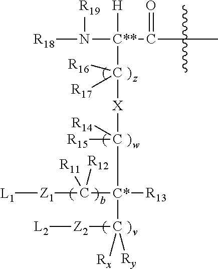

wherein the chiral centre is in the D configuration. A compound in this form may also be referred to as a D-Cys analogue stereoisomer of a compound of the invention. In some embodiments, moiety A of this stereoisomer of the compound may be depicted as:

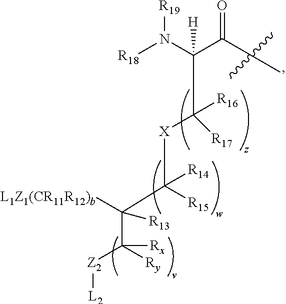

wherein L₁, L₂, Z₁, Z₂, Rₓ, R_{y}, R₁₁, R₁₂, R₁₃, R₁₄, R₁₅, R₁₆, R₁₇, R₁₈, R₁₉, b, v and z are as defined for the compound or Formula (I) and w is 1. Other stereocentres in these compounds may be racemic or independently enriched in either the R or S configuration.

In any aspect, the compound comprises a chiral centre in the Y moiety of the compound (shown at *):

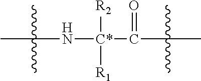

wherein the chiral centre is in the L-configuration. A compound in this form may also be referred to as an L-Y diastereomer of a compound described herein.

In any aspect, the compound comprises a chiral centre in the Y moiety of the compound (shown at *):

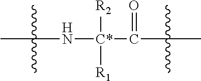

wherein the chiral centre is in the D-configuration. A compound in this form may also be referred to as a D-Y diastereomer of a compound described herein.

In any aspect, compositions comprising a compound of the invention (including a compound of any one of formulas (I)-(XIX)) or a pharmaceutically acceptable salt, solvate or prodrug thereof, and a pharmaceutically acceptable carrier, diluent or excipient may be used in a method or use of the invention.

In some embodiments, the compound as described herein is the R diastereomer around the chiral centre of the 2,3-bis(palmitoyloxy)propyl moiety of the compound.

In some embodiments, the compound as described herein is the S diastereomer around the chiral centre of the 2,3-bis(palmitoyloxy)propyl moiety of the compound.

In any aspect, a composition as described herein comprises a compound that is the R diastereomer around the chiral centre of the 2,3-bis(palmitoyloxy)propyl moiety of the compound.

In any aspect, a composition comprises a compound that is the S diastereomer around the chiral centre of the 2,3-bis(palmitoyloxy)propyl moiety of the compound.

In any aspect, 1%, 5%, 10%, 20%, 30%, 40%, 50%, 60%, 70%, 80%, 90%, 95%, 96%, 97%, 98%, 99% or more than 99% of the compound present in a composition is the R diastereomer around the chiral centre of the 2,3-bis(palmitoyloxy)propyl moiety of the compound.

In any aspect, 1%, 5%, 10%, 20%, 30%, 40%, 50%, 60%, 70%, 80%, 90%, 95%, 96%, 97%, 98%, 99% or more than 99% of the compound present in a composition is the S diastereomer around the chiral centre of the 2,3-bis(palmitoyloxy)propyl moiety of the compound (for example moiety A1).

In any aspect, the compound as described herein is the L diastereomer around the chiral centre of the cysteine analogue residue of the Pam2Cys analogue moiety compound (for example moiety Y).

In any aspect, the compound as described herein is the L diastereomer around the chiral centre of the cysteine residue of the Pam2Cys moiety compound (for example moiety Y).

In any aspect, the compound as described herein is the D diastereomer around the chiral centre of the cysteine analogue residue of the Pam2Cys analogue moiety compound (for example moiety Y).

In any aspect, the compound as described herein is the D diastereomer around the chiral centre of the cysteine residue of the Pam2Cys moiety of the compound (for example moiety Y).

In any aspect, a composition as described herein comprises a compound that is the L diastereomer around the chiral centre of the cysteine analogue residue of the Pam2Cys analogue moiety of the compound (for example moiety Y).

In any aspect, a composition as described herein comprises a compound that is the L diastereomer around the chiral centre of the cysteine residue of the Pam2Cys moiety of the compound (for example moiety Y).

In any aspect, a composition as described herein comprises a compound that is the D diastereomer around the chiral centre of the cysteine analogue residue of the Pam2Cys analogue moiety of the compound (for example moiety Y).

In any aspect, a composition as described herein comprises a compound that is the D diastereomer around the chiral centre of the cysteine residue of the Pam2Cys moiety of the compound (for example moiety Y).

In any aspect, 1%, 5%, 10%, 20%, 30%, 40%, 50%, 60%, 70%, 80%, 90%, 95%, 96%, 97%, 98%, 99% or more than 99% of the compound present in the composition is the L diastereomer around the chiral centre of the cysteine analogue residue of the Pam2Cys analogue moiety of the compound.

In any aspect, 1%, 5%, 10%, 20%, 30%, 40%, 50%, 60%, 70%, 80%, 90%, 95%, 96%, 97%, 98%, 99% or more than 99% of the compound present in the composition is the L diastereomer around the chiral centre of the cysteine residue of the Pam2Cys moiety of the compound.

In any aspect 1%, 5%, 10%, 20%, 30%, 40%, 50%, 60%, 70%, 80%, 90%, 95%, 96%, 97%, 98%, 99% or more than 99% of the compound present in the composition is the D diastereomer around the chiral centre of the cysteine analogue residue of the Pam2Cys analogue moiety of the compound.

In any aspect, 1%, 5%, 10%, 20%, 30%, 40%, 50%, 60%, 70%, 80%, 90%, 95%, 96%, 97%, 98%, 99% or more than 99% of the compound present in the composition is the D diastereomer around the chiral centre of the cysteine residue of the Pam2Cys moiety of the compound.

In any aspect, the compound of the invention as described herein is the L diastereomer around the chiral centre of the Y moiety.

In any aspect, the compound as described herein is the D diastereomer around the chiral centre of the Y moiety.

In any aspect, a composition as described herein comprises a compound that is the L diastereomer around the chiral centre of the Y moiety.

In any aspect, a composition as described herein comprises a compound that is the D diastereomer around the chiral centre of the Y moiety.

In any aspect, 1%, 5%, 10%, 20%, 30%, 40%, 50%, 60%, 70%, 80%, 90%, 95%, 96%, 97%, 98%, 99% or more than 99% of the compound present in the composition is the L diastereomer around the chiral centre of the Y moiety.

In any aspect, 1%, 5%, 10%, 20%, 30%, 40%, 50%, 60%, 70%, 80%, 90%, 95%, 96%, 97%, 98%, 99% or more than 99% of the compound present in the composition is the D diastereomer around the chiral centre of the Y moiety.

The compounds of formulas (I)-(XIX) described herein may demonstrate substantial stability in solution. This solution stability may be observed by storing solutions of the compounds under ambient storage conditions (eg at 25° C.) or under accelerated degradation stability (eg at 40° C.) for at least about 14 days.

In any aspect, any of the compounds described herein may be administered in the form of a pharmaceutically acceptable salt.

The term “pharmaceutically acceptable” may be used to describe any pharmaceutically acceptable salt, hydrate or prodrug, or any other compound which upon administration to a subject, is capable of providing (directly or indirectly) a compound of the invention as described herein, or a pharmaceutically acceptable salt, prodrug or ester thereof, or an active metabolite or residue thereof.

Suitable pharmaceutically acceptable salts may include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts may include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

In the case of compounds that are solids, it will be understood by those skilled in the art that the inventive compounds, agents and salts may exist in different crystalline or polymorphic forms, all of which are intended to be within the scope of the present invention and specified formulae.

The term “polymorph” includes any crystalline form of compounds of the invention as described herein, such as anhydrous forms, hydrous forms, solvate forms and mixed solvate forms.

Compounds of the invention described herein are intended to cover, where applicable, solvated as well as unsolvated forms of the compounds. Thus compounds of the invention described herein include compounds having the indicated structures, including the hydrated or solvated forms, as well as the non-hydrated and non-solvated forms.

As used herein, the term “solvate” refers to a complex of variable stoichiometry formed by a solute (in this invention, a compound of the invention described herein, or a pharmaceutically acceptable salt, prodrug or ester thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

The compounds as described herein are to also include isotope variations, such as the replacement of hydrogen for deuterium.

A “prodrug” is a compound that may not fully satisfy the structural requirements of the compounds provided herein, but is modified in vivo, following administration to a subject or patient, to produce a compound of the invention as described herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of any of compounds of Formulas (I)-(XIX). The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of the compounds described herein, including the compounds of formulas (I)-(XIX), or other structure as depicted herein.

The general chemical terms used in the formulae herein have their usual meaning.

The term “aliphatic” is intended to include saturated and unsaturated, nonaromatic, straight chain, branched, acyclic, and cyclic hydrocarbons. Those skilled in the art will appreciate that aliphatic groups include, for example, alkyl, alkenyl, alkynyl, cycloalkyl, and cycloalkenyl groups, and hybrids thereof such as (cycloalkyl)alkyl, (cycloalkenyl)alkyl and (cycloalkyl)alkenyl groups. In various embodiments, aliphatic groups comprise from 1-12, 1-8, 1-6, or 1-4 carbon atoms. In some embodiments, aliphatic groups comprise 5-21, from 9-21, or from 11-21 carbon atoms, such as from 11, 13, 15, 17, or 19 carbon atoms. In some embodiments, the aliphatic group is saturated.

The term “heteroaliphatic” is intended to include aliphatic groups, wherein one or more chain and/or ring carbon atoms are independently replaced with a heteroatom, preferably a heteroatom selected from oxygen, nitrogen and sulfur. In some embodiments, the heteroaliphatic is saturated. Examples of heteroaliphatic groups include linear or branched, heteroalkyl, heteroalkenyl, and heteroalkynyl groups.

The term “alkyl” is intended to include saturated straight chain and branched chain hydrocarbon groups. In some embodiments, alkyl groups have from 1 to 12, 1 to 10, 1 to 8, 1 to 6, or from 1 to 4 carbon atoms. In some embodiments, alkyl groups have from 5-21, from 9-21, or from 11-21 carbon atoms, such as from 11, 13, 15, 17, or 19 carbon atoms. Examples of straight chain alkyl groups include, but are not limited to, methyl, ethyl, n-propyl, n-butyl, n-pentyl, n-hexyl, n-heptyl, and n-octyl. Examples of branched alkyl groups include, but are not limited to, isopropyl, iso-butyl, sec-butyl, tert-butyl, neopentyl, isopentyl, and 2,2-dimethylpropyl.

The term “alkenyl” is intended to include straight and branched chain alkyl groups having at least one double bond between two carbon atoms. In some embodiments, alkenyl groups have from 2 to 12, from 2 to 10, from 2 to 8, from 2 to 6, or from 2 to 4 carbon atoms. In some embodiments, alkenyl groups have from 5-21, from 9-21, or from 11-21 carbon atoms, such as from 11, 13, 15, 17, or 19 carbon atoms. In some embodiments, alkenyl groups have one, two, or three carbon-carbon double bonds. Examples of alkenyl groups include, but are not limited to, vinyl, allyl, —CH═CH(CH₃), —CH═C(CH₃)₂, —C(CH₃)═CH₂, and —C(CH₃)═CH(CH₃).

The term “alkynyl” is intended to include straight and branched chain alkyl groups having at least one triple bond between two carbon atoms. In some embodiments, the alkynyl group have from 2 to 12, from 2 to 10, from 2 to 8, from 2 to 6, or from 2 to 4 carbon atoms. In some embodiments, alkynyl groups have one, two, or three carbon-carbon triple bonds. Examples include, but are not limited to, —C═CH, —C═CH₃, —CH₂C═CH₃, and —C═CH₂CH(CH₂CH₃)₂.

The term “heteroalkyl” is intended to include alkyl groups, wherein one or more chain carbon atoms are replaced with a heteroatom, preferably a heteroatom selected from the group consisting of oxygen, nitrogen, and sulfur. In some embodiments, the heteroalkyl is saturated. Heteroalkyl groups include, for example, polyethylene glycol groups and polyethylene glycol ether groups, and the like.

The term “cycloalkyl” is intended to include mono-, bi- or tricyclic alkyl groups. In some embodiments, cycloalkyl groups have from 3 to 12, from 3 to 10, from 3 to 8, from 3 to 6, from 3 to 5 carbon atoms in the ring(s). In some embodiments, cycloalkyl groups have 5 or 6 ring carbon atoms. Examples of monocyclic cycloalkyl groups include, but are not limited to, cyclopropyl, cyclobutyl, cyclopentyl, cyclohexyl, cycloheptyl, and cyclooctyl. In some embodiments, the cycloalkyl group has from 3 to 8, from 3 to 7, from 3 to 6, from 4 to 6, from 3 to 5, or from 4 to 5 ring carbon atoms. Bi- and tricyclic ring systems include bridged, spiro, and fused cycloalkyl ring systems. Examples of bi- and tricyclic ring cycloalkyl systems include, but are not limited to, bicyclo[2.1.1]hexanyl, bicyclo[2.2.1]heptanyl, adamantyl, and decalinyl.

The term “cycloalkenyl” is intended to include non-aromatic cycloalkyl groups having at least one double bond between two carbon atoms. In some embodiments, cycloalkenyl groups have one, two or three double bonds. In some embodiments, cycloalkenyl groups have from 4 to 14, from 5 to 14, from 5 to 10, from 5 to 8, or from 5 to 6 carbon atoms in the ring(s). In some embodiments, cycloalkenyl groups have 5, 6, 7, or 8 ring carbon atoms. Examples of cycloalkenyl groups include cyclohexenyl, cyclopentenyl, cyclohexadienyl, butadienyl, pentadienyl, and hexadienyl.

The term “aryl” is intended to include cyclic aromatic hydrocarbon groups that do not contain any ring heteroatoms. Aryl groups include monocyclic, bicyclic and tricyclic ring systems. Examples of aryl groups include, but are not limited to, phenyl, azulenyl, heptalenyl, biphenyl, fluorenyl, phenanthrenyl, anthracenyl, indenyl, indanyl, pentalenyl, and naphthyl. In some embodiments, aryl groups have from 6 to 14, from 6 to 12, or from 6 to 10 carbon atoms in the ring(s). In some embodiments, the aryl groups are phenyl or naphthyl. Aryl groups include aromatic-aliphatic fused ring systems. Examples include, but are not limited to, indanyl and tetrahydronaphthyl.

The term “heterocyclyl” is intended to include non-aromatic ring systems containing 3 or more ring atoms, of which one or more is a heteroatom. In some embodiments, the heteroatom is nitrogen, oxygen, or sulfur. In some embodiments, the heterocyclyl group contains one, two, three, or four heteroatoms. In some embodiments, heterocyclyl groups include mono-, bi- and tricyclic rings having from 3 to 16, from 3 to 14, from 3 to 12, from 3 to 10, from 3 to 8, or from 3 to 6 ring atoms. Heterocyclyl groups include partially unsaturated and saturated ring systems, for example, imidazolinyl and imidazolidinyl. Heterocyclyl groups include fused and bridged ring systems containing a heteroatom, for example, quinuclidyl. Heterocyclyl groups include, but are not limited to, aziridinyl, azetidinyl, azepanyl, diazepanyl, 1,3-dioxanyl, 1,3-dioxolanyl, isoxazolidinyl, morpholinyl, piperazinyl, piperidinyl, pyranyl, pyrazolidinyl, pyrrolinyl, pyrrolidinyl, tetrahydrofuranyl, tetrahydrothienyl, thiadiazolidinyl, and trithianyl.

The term “heteroaryl” is intended to include aromatic ring systems containing 5 or more ring atoms, of which, one or more is a heteroatom. In some embodiments, the heteroatom is nitrogen, oxygen, or sulfur. In some embodiments, heteroaryl groups include mono-, bi- and tricyclic ring systems having from 5 to 16, from 5 to 14, from 5 to 12, from 5 to 10, from 5 to 8, or from 5 to 6 ring atoms. Heteroaryl groups include, but are not limited to, pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, pyrazolopyridinyl, triazolopyridinyl, benzotriazolyl, benzoxazolyl, benzothiazolyl, imidazopyridinyl, isoxazolopyridinylxanthinyl, guaninyl, quinolinyl, isoquinolinyl, tetrahydroquinolinyl, quinoxalinyl, and quinazolinyl. Heteroaryl groups include fused ring systems in which all of the rings are aromatic, for example, indolyl, and fused ring systems in which only one of the rings is aromatic, for example, 2,3-dihydroindolyl.

The term “halo” or “halogen” is intended to include F, Cl, Br, and I.

The term “heteroatom” is intended to include oxygen, nitrogen, sulfur, or phosphorus. In some embodiments, the heteroatom is selected from the group consisting of oxygen, nitrogen, and sulfur.

As used herein, the term “substituted” is intended to mean that one or more hydrogen atoms in the group indicated is replaced with one or more independently selected suitable substituents, provided that the normal valency of each atom to which the substituent(s) are attached is not exceeded, and that the substitution results in a stable compound. In some embodiments, optional substituents in the compounds described herein include but are not limited to halo, CN, NO₂, OH, NH₂, NHR₁₀₀, NR₁₀₀R₂₀₀, C₁₋₆haloalkyl, C₁₋₆haloalkoxy, C(O)NH₂, C(O)NHR₁₀₀, C(O)NR₁₀₀R₂₀₀, SO₂R₁₀₀, OR₁₀₀, SR₁₀₀, S(O)R₁₀₀, C(O)R₁₀₀, and C₁₋₆aliphatic; wherein R₁₀₀ and R₂₀₀ are each independently C₁₋₆aliphatic, for example C₁₋₆alkyl.

Where a protecting group (PG) is referred to, a person skilled in the art would readily understand what type of protecting group would be suitable.

The term “amine protecting group” as used herein is intended to mean a group that is capable of being readily removed to provide the NH₂ group of an amine group and protects the amine group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, acyl and acyloxy groups, for example acetyl, chloroacetyl, trichloroacetyl, o-nitrophenylacetyl, o-nitrophenoxy-acetyl, trifluoroacetyl, acetoacetyl, 4-chlorobutyryl, isobutyryl, picolinoyl, aminocaproyl, benzoyl, methoxy-carbonyl, 9-fluorenylmethoxycarbonyl, 2,2,2-trifluoroethoxycarbonyl, 2-trimethylsilylethoxy-carbonyl, tert-butyloxycarbonyl, benzyloxycarbonyl, p-nitrobenzyloxycarbonyl, 2,4-dichloro-benzyloxycarbonyl, and the like. Further examples include Cbz (carboxybenzyl), Nosyl (o- or p-nitrophenylsulfonyl), Bpoc (2-(4-biphenyl)isopropoxycarbonyl) and Dde (1-(4,4-dimethyl-2,6-dioxohexylidene)ethyl). In some embodiments, the amine protecting groups for the purposes described herein include (but are not limited to) tert-butyloxycarbonyl (t-Boc) and 9H-fluoren-9-ylmethoxycarbonyl (Fmoc).

The term “carboxyl protecting group” as used herein is intended to mean a group that is capable of being readily removed to provide the OH group of a carboxyl group and protects the carboxyl group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, alkyl and silyl groups, for example methyl, ethyl, tert-butyl, methoxymethyl, 2,2,2-trichloroethyl, benzyl, diphenylmethyl, trimethylsilyl, and tert-butyldimethylsilyl, and the like.

The term “carboxamide protecting group” as used herein is intended to mean a group that is capable of being readily removed to provide the NH₂ group of a carboxamide group and protects the carboxamide group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, 9-xanthenyl (Xan), trityl (Trt), methyltrityl (Mtt), cyclopropyldimethylcarbinyl (Cpd), and dimethylcyclopropylmethyl (Dmcp).

The term “ester” refers to a carboxylic acid group where the hydrogen of the hydroxyl group has been replaced by a saturated, straight-chain (i.e. linear) or branched hydrocarbon group. Specific examples of alkyl groups are methyl, ethyl, propyl, iso-propyl, n-butyl, iso-butyl, sec-butyl, tert-butyl, n-pentyl, iso-pentyl, n-hexyl and 2,2-dimethylbutyl. The alkyl group may be a C₁-C₆ alkyl group. As used herein a wording defining the limits of a range of length such as, for example, “from 1 to 5” means any integer from 1 to 5, i.e. 1, 2, 3, 4 and 5. In other words, any range defined by two integers explicitly mentioned is meant to comprise and disclose any integer defining said limits and any integer comprised in said range. The alkyl group may be a branched alkyl group.

As used herein, ‘Ser’ refers to the amino acid serine and ‘Cys’ refers to the amino acid cysteine.

As used herein, ‘PEG’ refers to the polymer compound polyethylene glycol. Unless otherwise defined, reference to ‘PEG’ includes any length polymer of ethylene oxide. Reference to PEG also includes substituted PEG. In some embodiments, substituted PEG may be defined by formulas B-I or B-II as described herein.

As used herein, the term “and/or” means “and”, or “or”, or both.

The term “(s)” following a noun contemplates the singular and plural form, or both.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

It will be understood that compounds of the invention may possess a chiral centre and may therefore exist in an R- or S-configuration. The compounds may be provided in the form of a racemate or in an enatio- or diastereo-enriched form. Enantio- and diastereo-enriched forms of the compounds may be obtained either through asymmetric synthesis, the incorporation of chiral pool materials or through a stereoselective resolution. The compounds may therefore be provided as a purified enantiomer or diastereomer, or as a mixture of any ratio thereof. The isomers may be separated conventionally by chromatographic methods or using a resolving agent. Alternatively the individual isomers may be prepared by asymmetric synthesis using chiral intermediates. Where the compound has a carbon-carbon double bond, it may occur in Z- or E-form and all isomeric forms of the compounds being included in the present invention.

In some embodiments, the compound of the invention is selected from any of the following compounds:

| | Com- |
|---|---|
| | pound |
| Compound Structure | name |
| 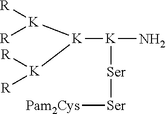 | 001 |
| 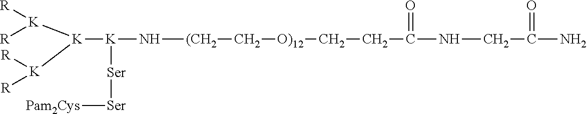 | 002 |
| 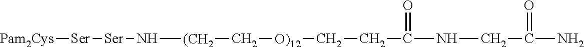 | 003 |
| Pam₂Cys-Ser-Ser-Lys-Lys-Lys-Lys | 004 |
| Pam₂Cys-Ser-Lys-Lys-Lys-Lys | 005 |
| 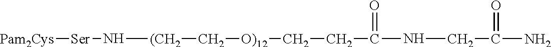 | A101 |
| 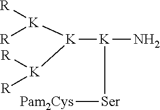 | 007 |
| 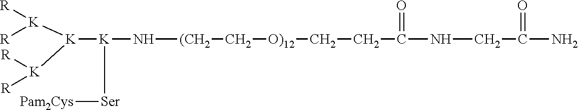 | 008 |
| 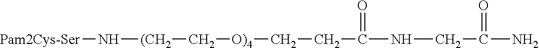 | 009 |
| 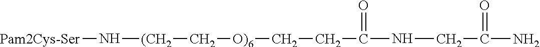 | 010 |
| 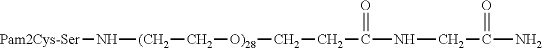 | A105 |
| 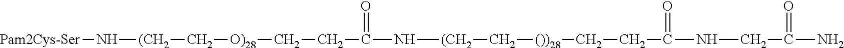 | A106 |
| 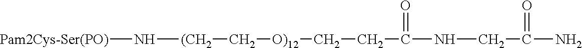 | A104 |
| 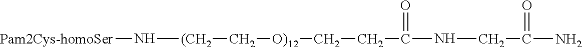 | A103 |
| 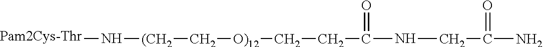 | A102 |
| 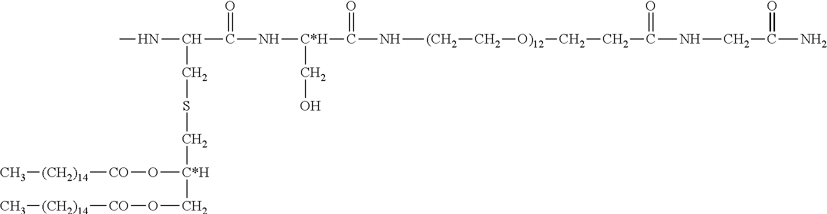 | A109 |
| 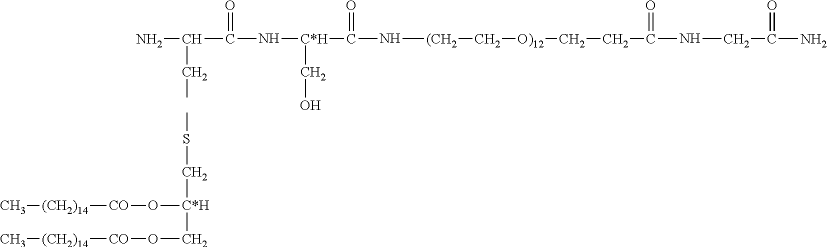 | A110 |
| 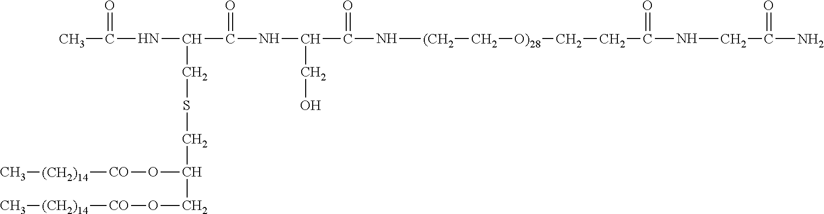 | A111 |
| 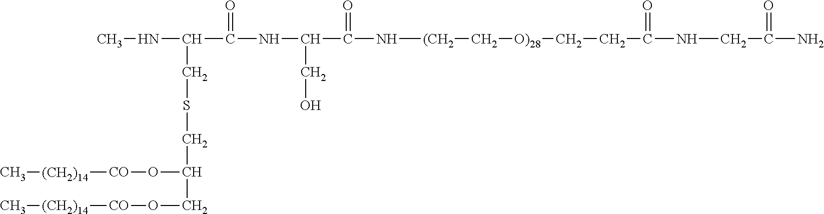 | A112 |
| 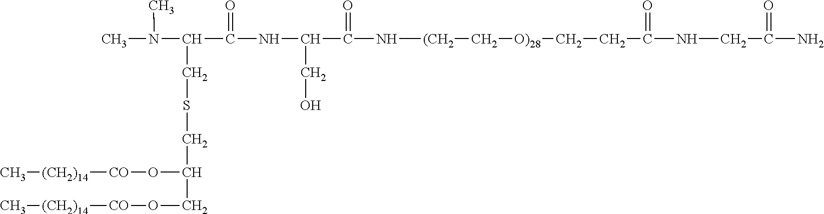 | A113 |
| 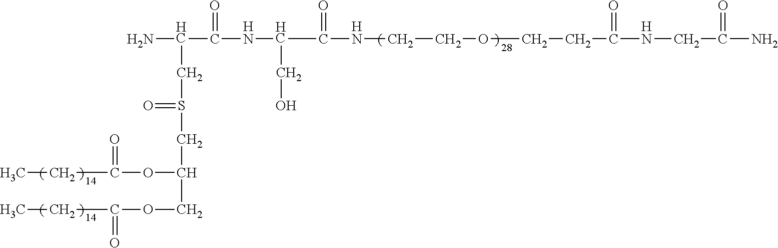 | A114 |
| 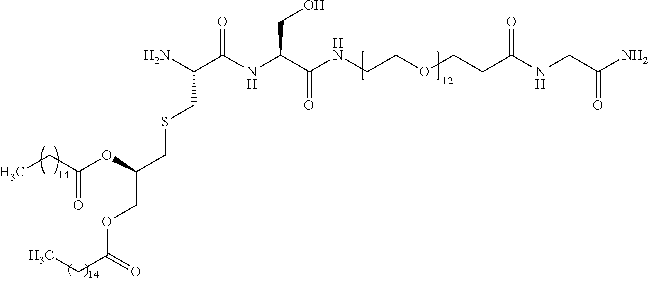 | A107 |
| 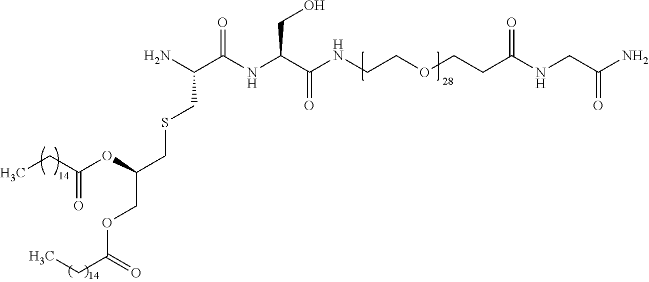 | A108 |
| 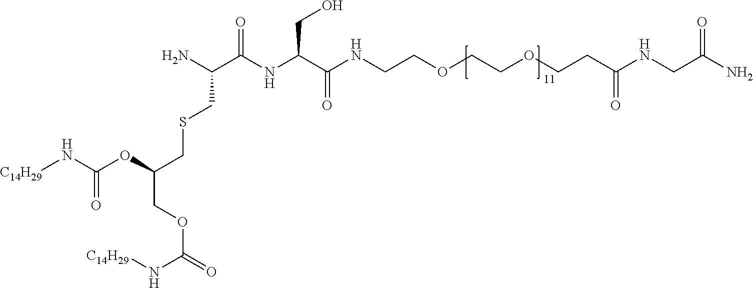 | A115 |
| 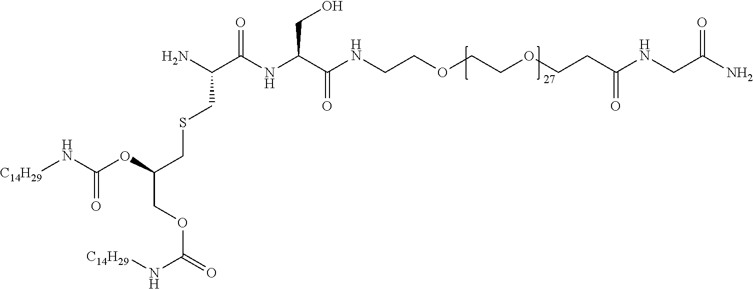 | A116 |
| 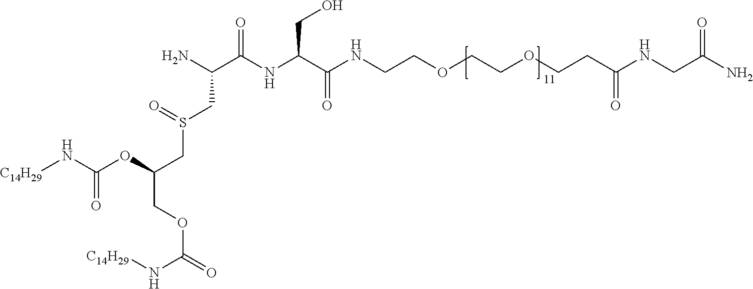 | A117 |
| 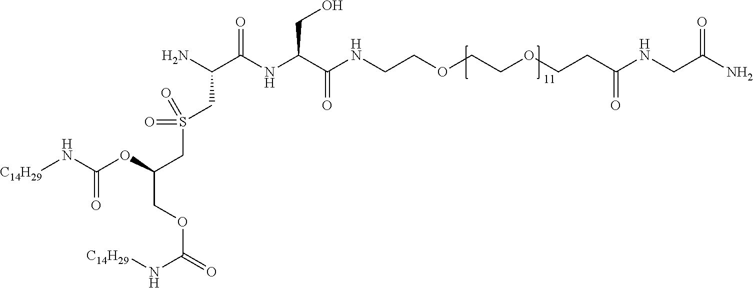 | A118 |
| 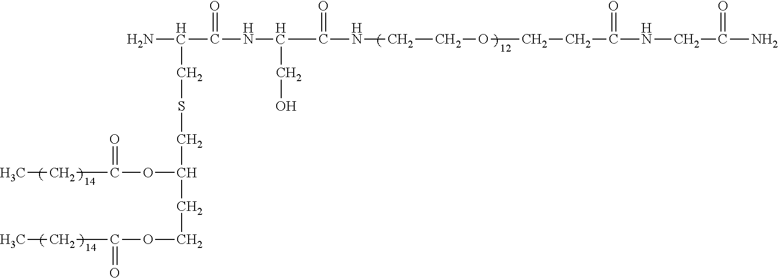 | A201 |
| 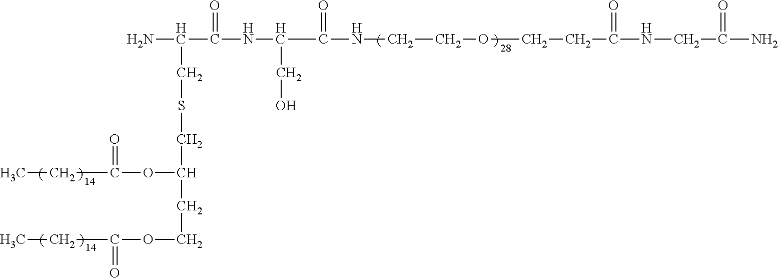 | A202 |
| 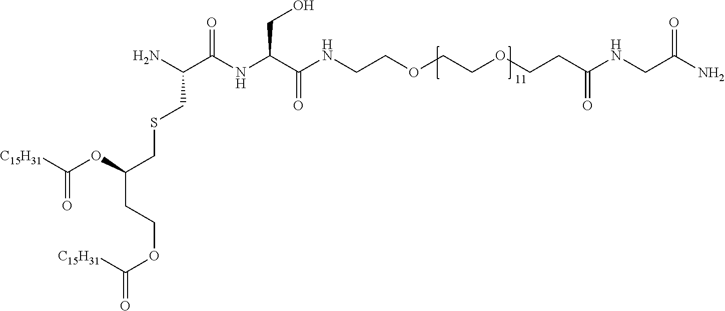 | A203 |
| 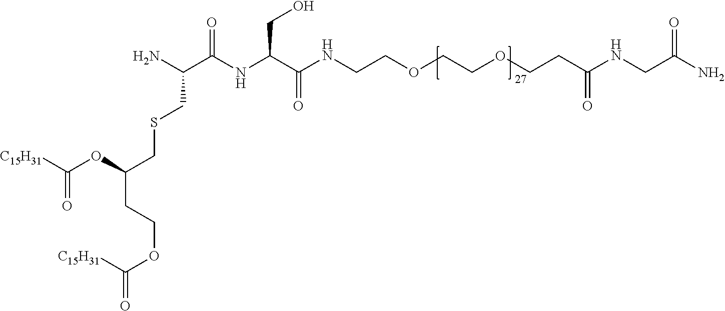 | A204 |
| 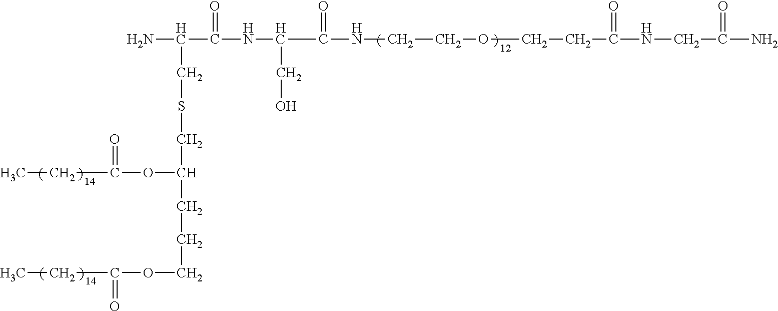 | A205 |
| 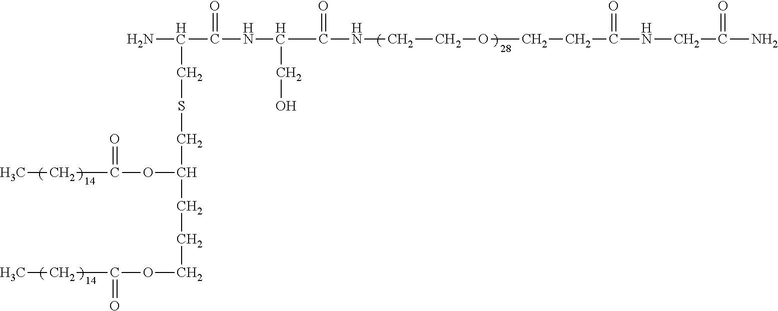 | A206 |
| 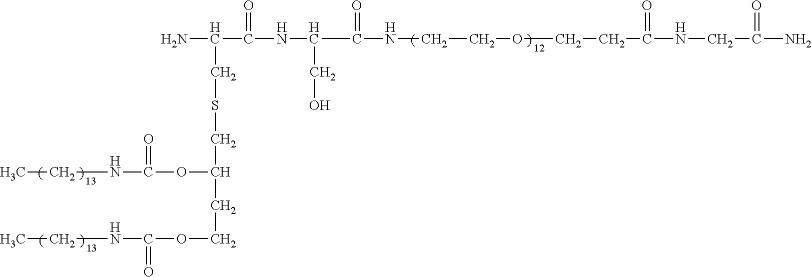 | A207 |
| 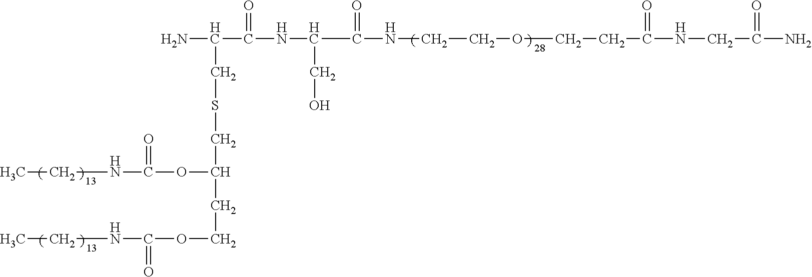 | A208 |
| 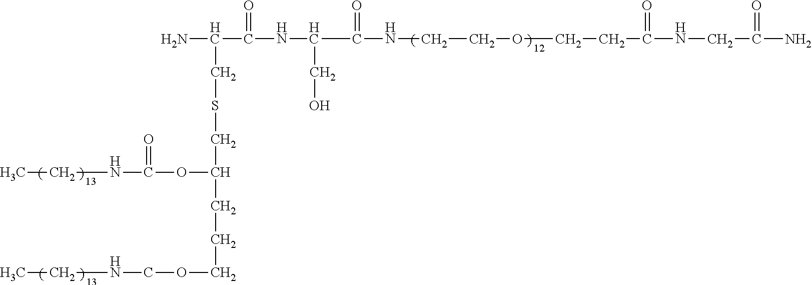 | A209 |
| 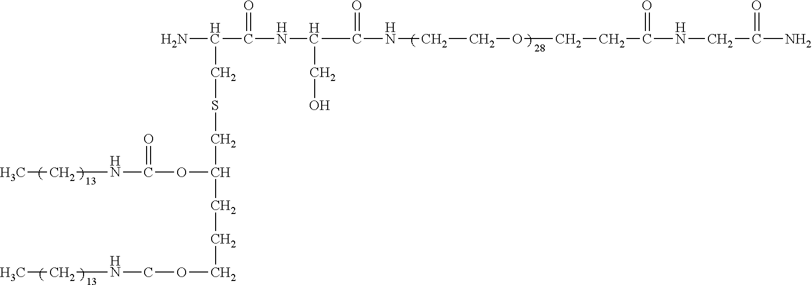 | A210 |
| 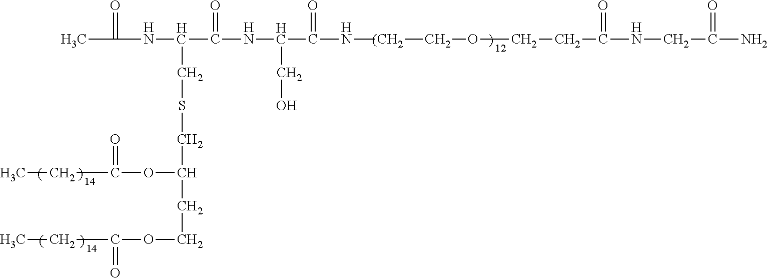 | A211 |
| 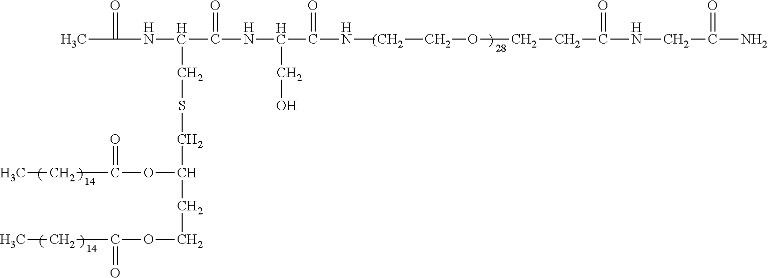 | A212 |
| 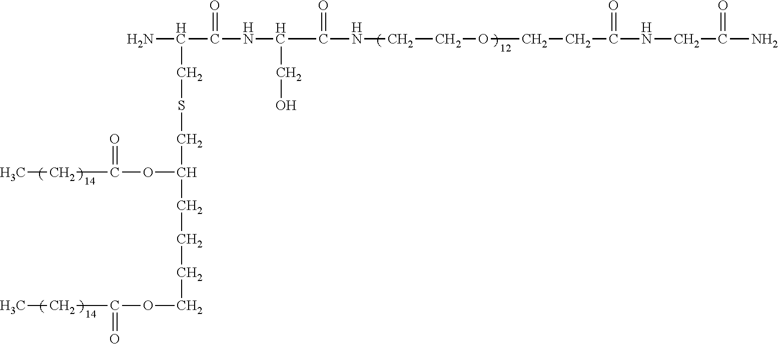 | A213 |
| 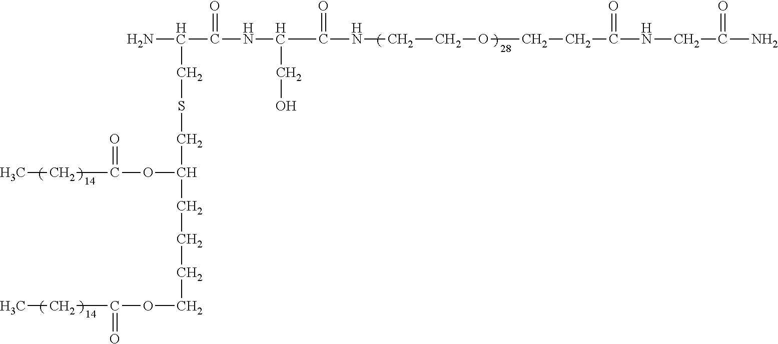 | A214 |
| 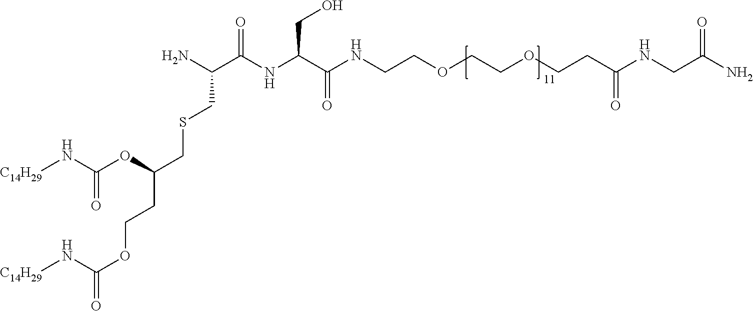 | A215 |
| 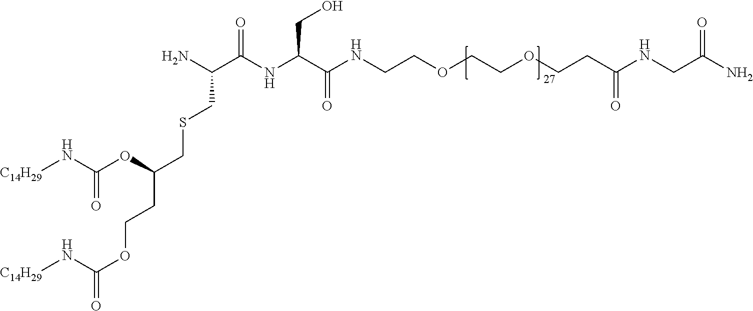 | A216 |
| 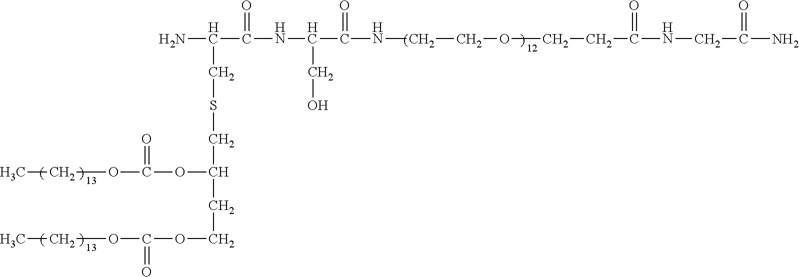 | A217 |
| 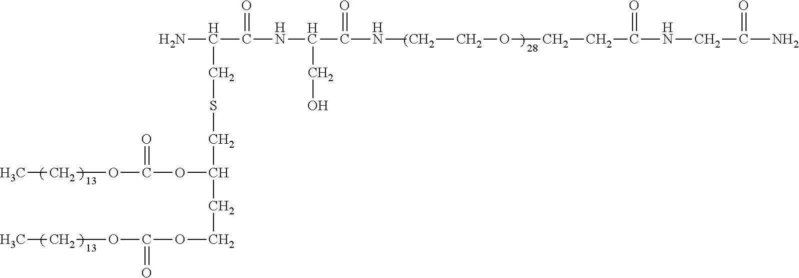 | A218 |
| 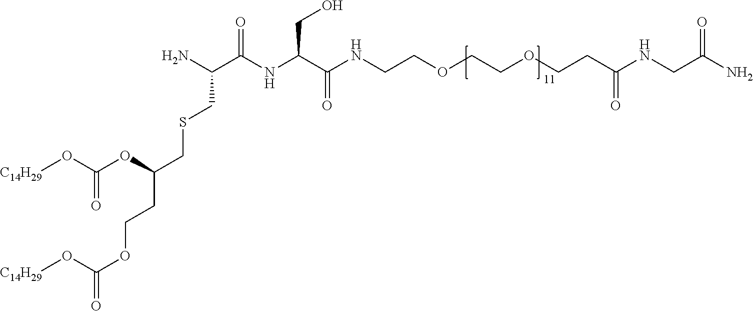 | A219 |
| 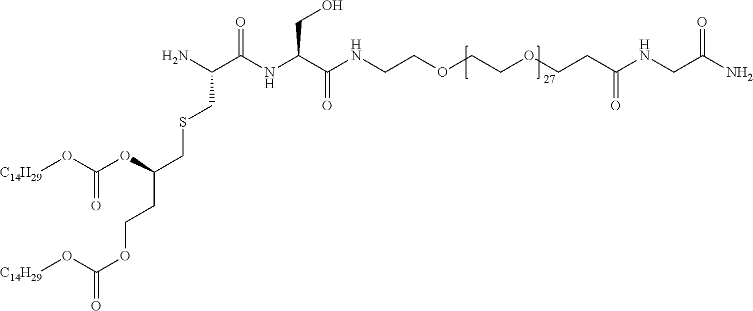 | A220 |
| 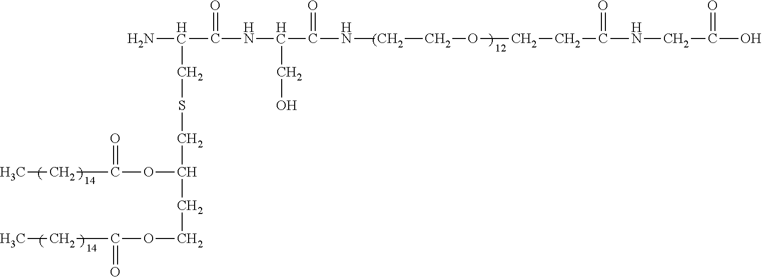 | A221 |
| 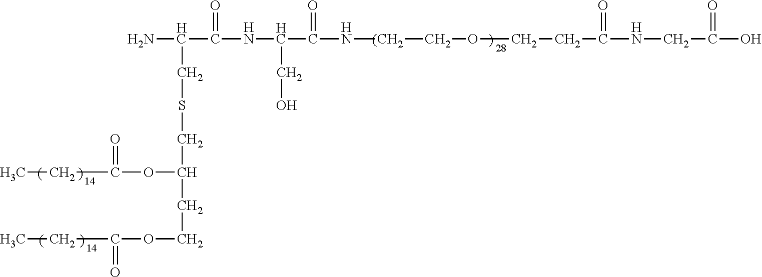 | A222 |
| 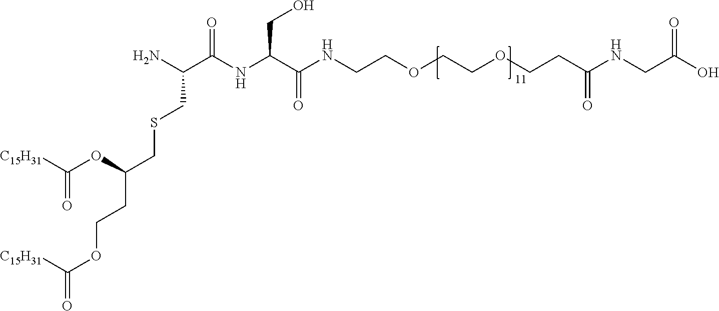 | A223 |
| 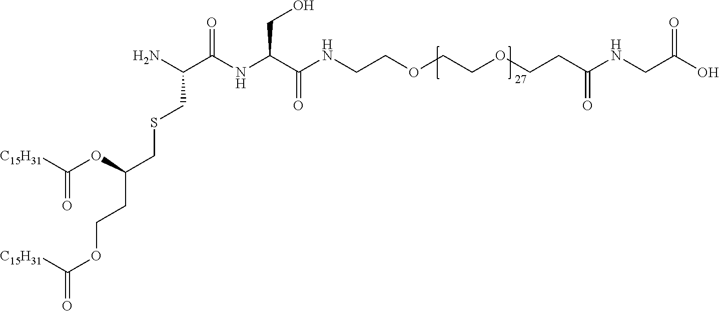 | A224 |
| 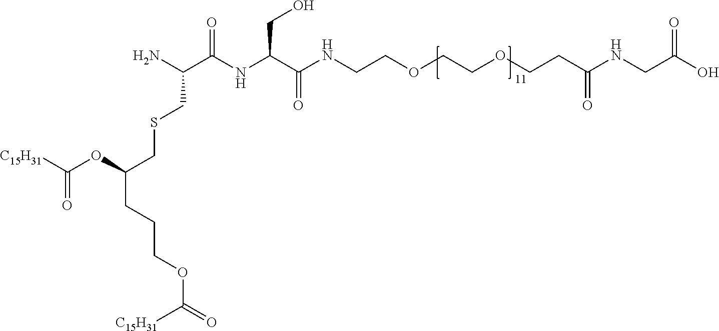 | A225 |
| 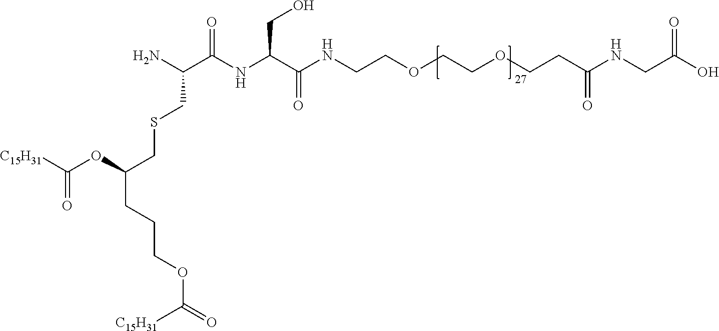 | A226 |
| 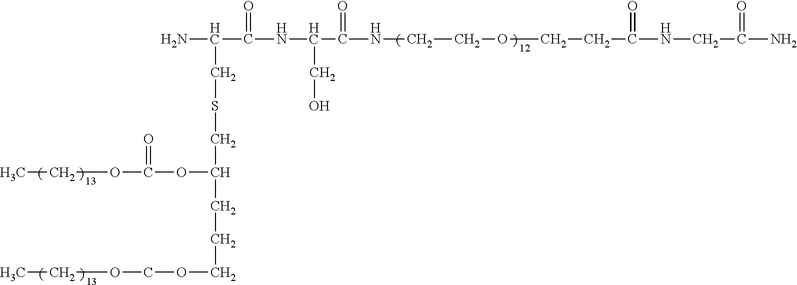 | A227 |
| 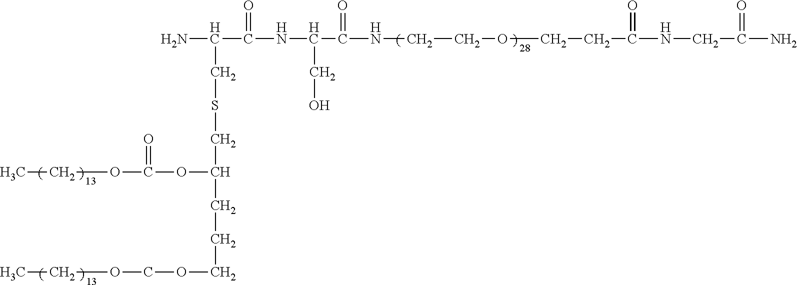 | A228 |
| 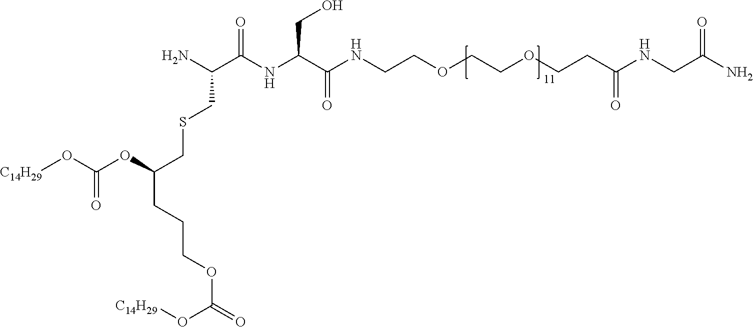 | A229 |
| 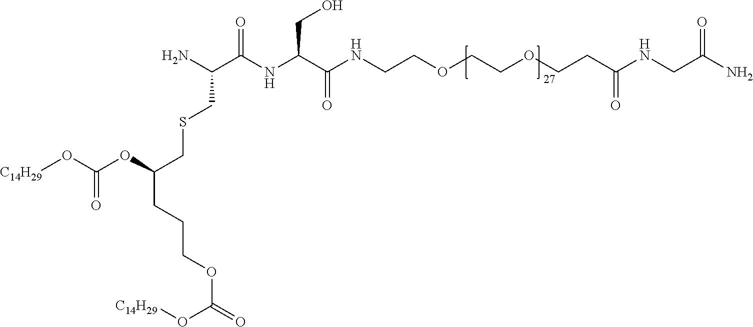 | A230 |
| 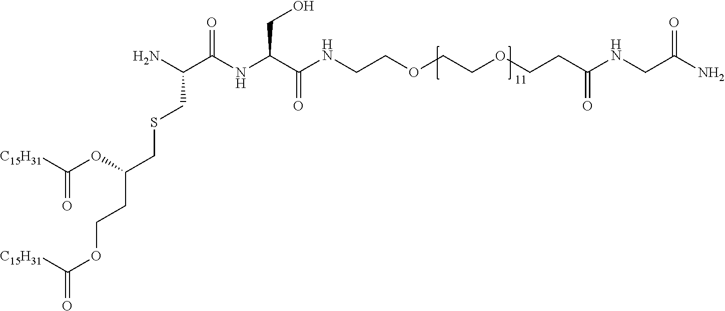 | A231 |
| 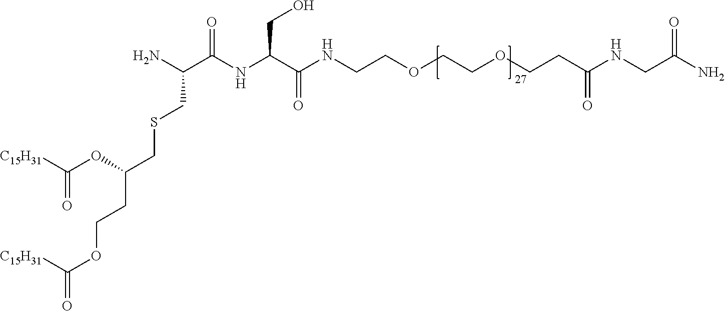 | A232 |

Compounds of the invention may be prepared by techniques known in the art. For example, compounds of the invention including any one of formulas (I)-(XIX) comprising an A1 moiety may be prepared by techniques described in WO2019/119067, the entire contents of which are hereby incorporated by reference, and by the techniques as described in Example 1. Compounds of the invention including any one of formulas (I)-(XIX) comprising an A2 moiety may be provided by techniques described in WO2020/257870, the entire contents of which are hereby incorporated by reference, and by the techniques as described in Example 1.

Pharmaceutical Compositions

The present invention also provides for compositions containing a compound of the invention as described herein or a pharmaceutically acceptable salt, solvate or prodrug thereof, and a pharmaceutically acceptable carrier, diluent or excipient. Any of the compounds described herein or variations thereof may be included in the compositions of the invention.

Pharmaceutical compositions may be formulated from compounds as described herein for any appropriate route of administration including, for example, topical (for example, transdermal or ocular), oral, buccal, respiratory (for example, nasal, inhalation, intrapulmonary), vaginal, rectal or parenteral administration. The term parenteral as used herein includes subcutaneous, intradermal, intravascular (for example, intravenous), intramuscular, spinal, intracranial, intrathecal, intraocular, periocular, intraorbital, intrasynovial and intraperitoneal injection, as well as any similar injection or infusion technique. Suitable oral forms include, for example, tablets, troches, lozenges, aqueous or oily suspensions, dispersible powders or granules, emulsions, hard or soft capsules, or syrups or elixirs. For intravenous, intramuscular, subcutaneous, or intraperitoneal administration, one or more compounds may be combined with a sterile aqueous solution which is preferably isotonic with the blood of the recipient. Such formulations may be prepared by dissolving solid active ingredient in water containing physiologically compatible substances such as sodium chloride or glycine, and having a buffered pH compatible with physiological conditions to produce an aqueous solution, and rendering said solution sterile. The formulations may be present in unit or multi-dose containers such as sealed ampoules or vials. Examples of components are described in Martindale—The Extra Pharmacopoeia (Pharmaceutical Press, London 1993) and Martin (ed.), Remington's Pharmaceutical Sciences. Preferably, the compositions are formulated for administration to the respiratory tract, for example, by intrapulmonary administration (eg. inhalation) or intranasal administration. The compositions may be administered to the upper and/or lower respiratory tract.

Preferably, the pharmaceutical compositions are in a form suitable for administration via the respiratory route, and may be in any form such as a powder, liquid or suspension. Such compositions may target tissue including pulmonary tissue (including alveolus, terminal bronchiole, bronchiole, and bronchus) or the nasal cavity (including paranasal cavity, frontal sinus, ethmoid sinus, maxillary sinus, sphenoidal sinus, superior turbinate, middle turbinate, and inferior turbinate).

Preferably the pharmaceutical compositions are formulated for intranasal administration or inhalation.

Methods of Prevention and Treatment

In one aspect, the present invention provides a method of treating and/or preventing a disease associated with a coronavirus, the method comprising raising an innate immune response in a subject by administering to the subject in need thereof a compound of the invention as described herein or a pharmaceutically acceptable salt, solvate or prodrug thereof, or pharmaceutical composition as described herein, thereby treating and/or preventing a disease associated with a coronavirus.

In another aspect, the present invention provides a method of treating and/or preventing a disease associated with, or caused by, a coronavirus, the method comprising administering to a subject in need thereof a compound of the invention as described herein or a pharmaceutically acceptable salt, solvate or prodrug thereof, or pharmaceutical composition as described herein, thereby treating and/or preventing a disease associated with, or caused by, a coronavirus.

In another aspect, the present invention provides a method of treating and/or preventing a respiratory disease or condition associated with a coronavirus infection, the method comprising administering to a subject in need thereof a compound of the invention as described herein or a pharmaceutically acceptable salt, solvate or prodrug thereof, or pharmaceutical composition as described herein, thereby treating and/or preventing a respiratory disease or condition associated with a coronavirus infection.

In another aspect, the present invention provides a method for reducing airway inflammation associated with, or caused by, a coronavirus, the method comprising administering to a subject in need thereof a compound of the invention as described herein or a pharmaceutically acceptable salt, solvate or prodrug thereof, or pharmaceutical composition as described herein, thereby reducing airway inflammation associated with, or caused by, a coronavirus.

In another aspect, the present invention also provides a method of improving the ability of a subject to control a respiratory disease or condition during a coronavirus infection, the method comprising administering to a subject in need thereof a compound of the invention as described herein or a pharmaceutically acceptable salt, solvate or prodrug thereof, or pharmaceutical composition as described herein, thereby improving the ability of a subject to control a respiratory disease or condition during a coronavirus infection.

In another aspect, the present invention provides for use of a compound of the invention as described herein or a pharmaceutically acceptable salt, solvate or prodrug thereof, or pharmaceutical composition as described herein, in the preparation of a medicament for raising an innate immune response in a subject diagnosed with, or suspected of having, a coronavirus infection.

In another aspect, the present invention provides for use of a compound of the invention as described herein or a pharmaceutically acceptable salt, solvate or prodrug thereof, or pharmaceutical composition as described herein, in the preparation of a medicament for treating and/or preventing a disease caused by a coronavirus.

In another aspect, the present invention further provides for use of a compound of the invention as described herein or a pharmaceutically acceptable salt, solvate or prodrug thereof, or pharmaceutical composition as described herein, in the preparation of a medicament for treating and/or preventing a respiratory disease or condition associated with a coronavirus infection in a subject.

In another aspect, the present invention further provides for use of a compound of the invention as described herein or a pharmaceutically acceptable salt, solvate or prodrug thereof, or pharmaceutical composition as described herein, in the preparation of a medicament for treating and/or preventing a coronavirus infection in a subject.

In another aspect, the present invention further provides use of a compound of the invention as described herein or a pharmaceutically acceptable salt, solvate or prodrug thereof, or pharmaceutical composition as described herein, in the preparation of a medicament for reducing airway inflammation in a subject diagnosed with, or suspected of having, a coronavirus infection.

In another aspect, the present invention further provides use of a compound of the invention as described herein or a pharmaceutically acceptable salt, solvate or prodrug thereof, or pharmaceutical composition as described herein, in the preparation of a medicament for improving the ability of a subject to control a respiratory disease or condition during a coronavirus infection.

In one aspect, the present invention provides for a compound of the invention as described herein or a pharmaceutically acceptable salt, solvate or prodrug thereof, or pharmaceutical composition as described herein, for use in raising an innate immune response in a subject diagnosed with, or suspected of having, a coronavirus infection.

In another aspect, the present invention provides for a compound of the invention as described herein or a pharmaceutically acceptable salt, solvate or prodrug thereof, or pharmaceutical composition as described herein, for use in treating and/or preventing a disease caused by a coronavirus in a subject.

In another aspect, the present invention provides for a compound of the invention as described herein or a pharmaceutically acceptable salt, solvate or prodrug thereof, or pharmaceutical composition as described herein, for use in treating and/or preventing a respiratory disease or condition associated with a coronavirus infection in a subject.

In another aspect, the invention provides a compound of the invention as described herein or a pharmaceutically acceptable salt, solvate or prodrug thereof, or pharmaceutical composition as described herein, for use in reducing airway inflammation in a subject diagnosed with, or suspected of having, a coronavirus infection.

In another aspect, the invention provides a compound of the invention as described herein or a pharmaceutically acceptable salt, solvate or prodrug thereof, or pharmaceutical composition as described herein, for use in controlling a respiratory disease or condition during a coronavirus infection in a subject.

In any of these aspects, the compound may be administered in a composition. Typically, the composition further comprises a pharmaceutically acceptable carrier, diluent or excipient. The composition may be formulated for administration to the upper and/or lower respiratory tract, for example by inhalation or intranasally.

In any aspect of the invention, the compound of the invention as described herein or a pharmaceutically acceptable salt, solvate or prodrug thereof may be conjugated with other compounds. Other compounds are any of those described herein.

In any aspect of the invention, the compound of the invention as described herein or a pharmaceutically acceptable salt, solvate or prodrug thereof is administered once daily or once weekly.

In any aspect of the invention, where prevention or prophylaxis is intended or required, the compound is administered to the subject before any clinically or biochemically detectable symptoms of viral infection.

In any aspect of the invention, administration of the compound of the invention as described herein or a pharmaceutically acceptable salt, solvate or prodrug thereof to a subject reduces viral load in a subject. Preferably, the viral load is reduced in the respiratory tract, for example the upper and/or lower respiratory tract. Preferably, the viral load is reduced in the nasal cavity and pharynx (i.e throat).

In any aspect of the invention, administration of the compound of the invention as described herein or a pharmaceutically acceptable salt, solvate or prodrug thereof to a subject asymptomatic for coronavirus infection may prevent or reduce the progression to symptomatic phase.

In another aspect, the present invention provides a method of reducing the severity of a coronavirus infection, or reducing the period in which a subject displays one or more symptoms of a coronavirus infection, the method comprising administering to a subject in need thereof a compound of the invention as described herein or a pharmaceutically acceptable salt, solvate or prodrug thereof, or pharmaceutical composition as described herein, thereby reducing the severity of a coronavirus infection, or reducing the period in which a subject displays one or more symptoms of a coronavirus infection.

A reduction in coronavirus infection may be determined using any method known in the art or described herein, including measuring viral load in a sample from the subject after treatment and comparing it to viral load in a sample from the same subject before treatment. Preferably, the sample is taken from the respiratory tract, preferably the upper respiratory tract, for example the nose or pharynx (i.e. throat).

The term ‘respiratory disease’ or ‘respiratory condition’ refers to any one of several ailments that involve inflammation and affect a component of the respiratory system including the upper (including the nasal cavity, pharynx and larynx) and lower respiratory tract (including trachea, bronchi and lungs). The inflammation in the upper and lower respiratory tract may be associated with or caused by viral infection.

A symptom of respiratory disease may include cough, excess sputum production, a sense of breathlessness or chest tightness with audible wheeze.

The existence of, improvement in, treatment of or prevention of a respiratory disease may be determined by any clinically or biochemically relevant method of the subject or a biopsy therefrom. For example, a parameter measured may be the presence or degree of lung function, signs and symptoms of obstruction; exercise tolerance; night time awakenings; days lost to school or work; bronchodilator usage; Inhaled corticosteroid (ICS) dose; oral glucocorticoid (GC) usage; need for other medications; need for medical treatment; hospital admission.

As used herein, the term respiratory infection means an infection by a coronavirus anywhere in the respiratory tract.

An individual may be identified as having a respiratory tract infection by viral testing and may exhibit symptoms of itchy watery eyes, nasal discharge, nasal congestion, sneezing, sore throat, cough, headache, fever, malaise, fatigue and weakness. In one aspect, a subject having a respiratory infection may not have any other respiratory condition. Detection of the presence or amount of virus may be by PCR/sequencing of RNA isolated from clinical samples (nasal wash, sputum, BAL) or serology.

Route of Administration and Dosage

In the context of this specification the term “administering” and variations of that term including “administer” and “administration”, includes contacting, applying, delivering or providing a compound or composition as described herein to an organism, or a surface by any appropriate means.

The dose of the biologically active compound according to the invention may vary within wide limits and may be adjusted to individual requirements. Active compounds as described herein are generally administered in a therapeutically effective amount.

A composition according to the present invention is to be administered in an effective amount. The phrase ‘therapeutically effective amount’ or ‘effective amount’ generally refers to an amount of a compound of the invention described herein, a pharmaceutically acceptable salt, polymorph or prodrug thereof of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. Undesirable effects, e.g. side effects, are sometimes manifested along with the desired therapeutic effect; hence, a practitioner balances the potential benefits against the potential risks in determining what is an appropriate “effective amount”.

The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation. In one aspect, the dose administered to a subject is any dose that reduces viral load. Preferably, the dose does not significantly increase inflammation, for example does not significantly increase absolute neutrophil numbers or the proportion of neutrophils of total BAL cells. The terms “therapeutically effective amount” or “effective amount” may also refer to an amount of the compound of Formula (I), Formula (II), Formula (III), Formula (IV) and/or Formula (V) or a pharmaceutically acceptable salt, solvate or prodrug thereof, that results in an improvement or remediation of the symptoms of a respiratory infection, or respiratory disease or condition associated with a coronavirus infection.

In some embodiments, an effective amount for a human subject lies in the range of about 250 nmoles/kg body weight/dose to 0.005 nmoles/kg body weight/dose. Preferably, the range is about 250 nmoles/kg body weight/dose to 0.05 nmoles/kg body weight/dose. In some embodiments, the body weight/dose range is about 250 nmoles/kg, to 0.1 nmoles/kg, about 50 nmoles/kg to 0.1 nmoles/kg, about 5 nmoles/kg to 0.1 nmol/kg, about 2.5 nmoles/kg to 0.25 nmoles/kg, or about 0.5 nmoles/kg to 0.1 nmoles/kg body weight/dose. In some embodiments, the amount is at, or about, 250 nmoles, 50 nmoles, nmoles, 2.5 nmoles, 0.5 nmoles, 0.25 nmoles, 0.1 nmoles or 0.05 nmoles/kg body weight/dose of the compound. Dosage regimes are adjusted to suit the exigencies of the situation and may be adjusted to produce the optimum therapeutic dose.

Compounds of the invention as described herein may be included in compositions formulated as inhaled formulations, including dry powder, sprays, mists, or aerosols. This may be particularly preferred for treatment of a respiratory infection. For inhalation formulations, the composition or combination provided herein may be delivered via any inhalation methods known to a person skilled in the art. Such inhalation methods and devices include, but are not limited to, metered dose inhalers with propellants such as CFC or HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and may be filled into conventional aerosol containers that are closed by a suitable metering valve.

Inhalant compositions may comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent such as isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable formulations, wherein the carrier is a liquid, for administration, as for example, a nasal spray or as nasal drops, include aqueous or oily solutions of the active ingredient. Alternatively, the composition may be a dry powder and administered to the respiratory tract as defined herein.

It will be understood, however, that the specific dose level for any particular subject will depend upon a variety of factors including the activity of the specific compound employed, the age, body weight, general health, sex, diet, time of administration, route of administration, and rate of excretion, drug combination (i.e. other drugs being used to treat the subject), and the severity of the particular disorder undergoing therapy. The dosage will generally be lower if the compounds are administered locally rather than systemically, and for prevention rather than for treatment. Such treatments may be administered as often as necessary and for the period of time judged necessary by the treating physician. A person skilled in the art will appreciate that the dosage regime or therapeutically effective amount of the compound of the invention, or a pharmaceutically acceptable salt, solvate or prodrug thereof, to be administered may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in a single or multiple doses per day.

It will also be appreciated that different dosages may be required for treating different disorders.

As used herein, the terms “treatment” or “treating” of a subject includes the application or administration of a compound or composition of the invention to a subject (or application or administration of a compound of the invention to a cell or tissue from a subject) with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; or making the final point of degeneration less debilitating,

As used herein, “preventing” or “prevention” is intended to refer to at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease). Biological and physiological parameters for identifying such patients are provided herein and are also well known by physicians.

It will be understood that the invention disclosed and defined in this specification extends to all alternative combinations of two or more of the individual features mentioned or evident from the text or drawings. All of these different combinations constitute various alternative aspects of the invention.

### Example 1—Synthesis of Compounds

### Example 1.1—Synthesis a Using Fmoc Solid Phase Chemistry

Compounds of the invention including any one of formulas (I)-(XIX) comprising an A2 moiety may be provided by coupling a compound of the formula A2-I:

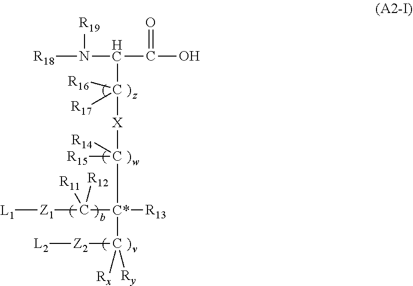

wherein L₁, L₂, Z₁, Z₂, v, b, w, z, Rₓ, R_{y}, R₁₁, R₁₂, R₁₃, R₁₄, R₁₅, R₁₆, R₁₇, R₁₈ and X have the meanings as defined for any compound of the invention defined herein and R₁₉ is an amino protecting group

with a compound of formula (YB-I):

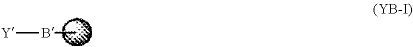

wherein

Y′ is

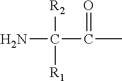

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

B′ is a Polyethylene Glycol (PEG); and

[symbol] is a solid support resin.

In some embodiments, B′ comprises a substituted PEG of Formula B-I. In these embodiments, the following sequence of solid phase reactions may be employed:
a) Optionally coupling 1 to 10 alpha amino acids or compounds derived from a natural alpha amino acid, that constitutes L, to a solid phase resin using Fmoc chemistry
b) Coupling PG-NH—(CH₂)ₚ—O—(CH₂CH₂O)ₙ—(CH₂)ₘ—COOH to a solid phase resin or substituted resin if L is present, wherein PG represents an amino protecting group compatible with Fmoc chemistry;
c) Removing PG;
d) Coupling PG-NH—CR₁₃R₁₄—COOH, wherein PG′ represents an amino protecting group compatible with Fmoc chemistry;
e) Removing PG′;
f) Coupling an acid of the formula (A-I);
g) Optionally removing R₁₉ and optionally acylating and/or alkylating to introduce R₁₈ and/or R₁₉; and
h) Removing the compound from the solid phase support

In some embodiments, B′ comprises a substituted PEG according to formula (B-II) and the following sequence of solid phase reactions may be employed:
a) Optionally coupling 1 to 10 alpha amino acids or compounds derived from a natural alpha amino acid, that constitute L, to a solid phase resin using Fmoc chemistry
b) Coupling PG-NH—(CH₂)ₜ—O—(CH₂CH₂O)ₖ—(CH₂)ₕ—COOH to a solid phase resin or substituted resin if L is present, wherein PG represents an amino protecting group compatible with Fmoc chemistry;
c) Removing PG;
d) Coupling PG′-NH—(CH₂)ₚ—O—(CH₂CH₂O)ₙ—(CH₂)ₘ—COOH, wherein PG′ represents an amino protecting group compatible with Fmoc chemistry;
e) Removing PG′;
f) Coupling PG″-NH—CR₁₃R₁₄—COOH, wherein PG″ represents an amino protecting group compatible with Fmoc chemistry;
g) Removing PG″;
h) Coupling an acid of the formula (A-I);
i) Optionally removing R₁₉ and optionally acylating and/or alkylating to incorporate R₁₈ and/or R₁₉; and
j) Removing the compound from the solid phase resin.

It will be appreciated that the exact sequence of events can be varied from that outlined, and additional steps added where necessary and synthetically expedient, for example oxidation of the cysteine sulfur to the sulfoxide or sulfone.

### Example 1.2—Synthesis of Intermediate for Use in the Solid Phase Coupling A

In some embodiments, the compound of formula A2-I is provided in the form of a compound of formula A2-II:

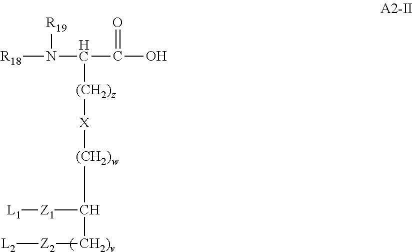

wherein L₁, L₂, X, v, w and R₁₈ are as defined for the compound of formula A-I above, Z₁ and Z₂ are independently selected from —NHC(O)—, —C(O)NH—, —OC(O)—, —C(O)O—, —NHC(O)O— and —OC(O)O—.

The compound of formula A2-II may be prepared by the synthesis shown in Scheme 1.

Scheme 1 describes the synthesis of embodiments of the compound of formula A2-II, wherein

X is S,

L₁-Z₁ are —OC(O)E-(CH₂)_{g}—CH₃, wherein E is —O— or —NH— and g is 10, 11, 12, 13, 14, 15, 16, 17 or 18;

L₂-Z₂ are —OC(O)E-(CH₂)_{g}—CH₃, wherein E is —O— or —NH— and g is 10, 11, 12, 13, 14, 15, 16, 17 or 18; and

R₁₉ is PG3, which is an amino protecting group.

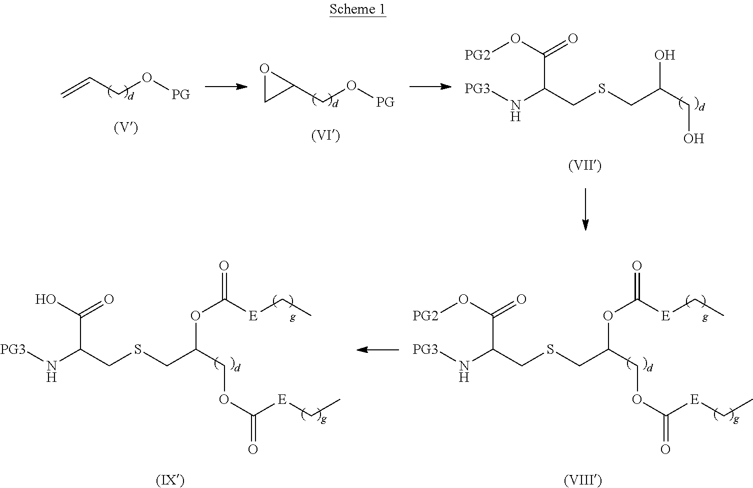

Reaction of protected alkene alcohols of the formula (V′), where PG is a suitable protecting group, for example a silyl group such as TBDMS, forms an epoxide of the formula (VI′). It will be appreciated that the epoxide formation maybe carried out to give the product racemically or to give enantioenriched material. If a racemic or scalemic mixture of enantiomers is produced preparative chiral chromatography is employed to separate the enantiomers if required.

Epoxides of the formula (VI′) are reacted with suitably protected cystine analogues, for example tert-butyl N-(((9H-fluoren-9-yl)methoxy)carbonyl)-S—(((R)-2-((((9H-fluoren-9-yl)methoxy)carbonyl)amino)-3-(tert-butoxy)-3-oxopropyl)thio)-D-cysteinate, where PG2 is a tert-butyl ester and PG3 is Fmoc, under reducing conditions to give alcohols of the formula (VII′). It will be appreciated that alcohols of the formula (VII′) can be comprised of more than one stereoisomer and where stereoisomers are present these can be separated by chiral preparative chromatography as required.

Alcohols of the formula (VII′) can be acylated to give carbonyl containing adducts of the formula (VIII′) using suitable reagents. Where esters are required, acid chlorides can be reacted in the presence of suitable bases and solvents; where carbamates are required isocyanates can be reacted in the presence of suitable bases and solvents and where carbonates are required chloroformates can be reacted in the presence of suitable bases and solvents. Carbonyl containing adducts of the formula (VIII′) can then be deprotected to reveal carboxylic acids of the formula (IX′) using suitable reagents, for example where PG2 is tert-butyl, trifluoroacetic acid can be used to preferentially remove the tert-butyl group.

Acids of the formula (IX′) can then be used as reagents in solid phase synthesis to add groups of formula Y and B.

### Example 1.3—Synthesis B Using Fmoc Solid Phase Chemistry

Compounds of the invention including any one of formulas (I)-(XIX) comprising an A2 moiety wherein z is 1, w is 1 and b is 0, may be provided by preparing a resin bound peptide of the following formula:

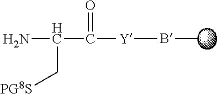

wherein

Y′ is

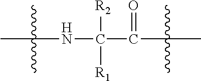

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

B′ is a Polyethylene Glycol (PEG);

PG^{s} is H or a sulphur protecting group, such as tert-butyl; and

[symbol] is a solid support resin.

Following optional sulphur deprotection, this resin bound peptide may be reacted with a 1,2-epoxy-alkanol of the following formula:

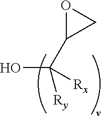

wherein Rₓ, R_{y} and v have the meanings given for Formula (I)

to provide an alkylated thiol of formula S-1:

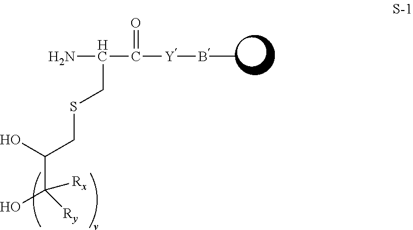

wherein Y′ and B′ have the meaning given above, and v has the meaning given for the compound of formula (I), or a sulfone or sulfoxide thereof.

The diol moieties of resin bound compound S-1 may be further reacted to provide a compound of the invention, for example, by diol functionalisation with palmitic groups or lauryl carbamate groups, etc.

### Example 1.4—Representative Synthesis and Characterisation of Selected Compounds of the Invention: A107, A108, A115, A116, A117, A118, A203, A204, A215 and A216

### Synthesis of Compounds A107 (x=11) and A108 (x=27)

Fmoc S-2,3-di(palmitoloxypropyl)-cysteine (S-Fmoc-Dpc-OH) was purchased from Bachem Inc.

Coupling of S-Fmoc-Dpc-OH to resin-bound peptide: Fmoc-Dpc-OH (100 mg, 0.24 mmol) is activated in DCM and DMF (1:1, v/v, 3 mL) with HOBt (36 mg, 0.24 mmol) and N,N′-diisopropylcarbodiimide (DICI; 37 uL, 0.24 mmol) at 0° C. for 5 min. The mixture is then added to a vessel containing the Boc-Cys-Ser(tBu)CH₂CH₂O-(PEG)₁₁-CH₂CH₂C(O)Gly resin or Boc-Cys-Ser(tBu)-CH₂CH₂O—(PEG)₂₇-CH₂CH₂C(O)Gly resin (0.25 mmol/g, 0.25 g=0.0625 mmole). After shaking for 2 h the solution is removed by filtration on a glass sinter funnel (porosity 3) and the resin washed with DCM and DMF (3×30 mL each). The reaction is monitored for completion using the trinitrobenezene sulfonic acid (TNBSA) test. If necessary a double coupling is performed.

Cleavage of peptide from the solid support: Reagent B (93% TFA, 5% water and 2% triisopropylsilane) for two hours. The peptide did not precipitate in chilled ether. Most of the TFA must be removed and then the residue is dissolved in 50% acetonitrile and purified immediately or freeze-dried.

### Synthesis of Compounds A115 and A116

Syntheses of compounds A115 (x=11) and A116 (x=27) were carried out as depicted in Scheme 2. (R)-glycidol is coupled to the thiol group of the cysteine residue attached to the peptide resin by alkylation; To 250 mg of Boc-Cys-Ser(tBu)CH₂CH₂O—(PEG)₁₁-CH₂CH₂C(O)Gly resin or Boc-Cys-Ser(tBu)CH₂CH₂O-PEG₂₇-CH₂CH₂C(O)Gly resin (0.25 mmole/g, 0.25 g=0.0625 mmole) saturated in DMF was added 250 μl of R-(+)-glycidol (MW=74.08, d=1.1, 250 μl=3.71 mmol, 60 fold excess over the free sulfhydryl group on the peptide resin) and 25 □l of diisopropylethylamine (DIPEA, MW=129.2, d=0.74, 25 μl=0.14 mmol). The reaction mixture was held at 50° C. for 2 hrs in a water bath and then the solid support then thoroughly washed with DMF. To 250 mg of the peptide resin washed with toluene following glycidolation, were added 100 μl of ethylmethylsulfide (W=76.16, d=0.842, 100 μl=1.10 mmole) followed by 105 μl of tetradecyl isocyanate (MW=239, d=0.869, 105 μl=0.38 mmol, i.e. 3-fold excess over each of the hydroxyl groups present on the solid support) and finally 210 μl of dibutyltin dilaurate (MW=631.6, d=1.053, 210 μl=0.35 mmol). The reaction mixture was sparged with nitrogen gas for approximately 5 min and mixed (Intelli—Mixer, RM-2, program F26 used) overnight at room temperature. The reaction mixture was transferred to a 50 ml tube and chloroform added to 50 ml. Following sonication for approximately 5 mins the white precipitate, formed during the reaction, dissolved. The solid support was washed with DMF and acetonitrile and the final product obtained following cleavage from the support was purified by HPLC.

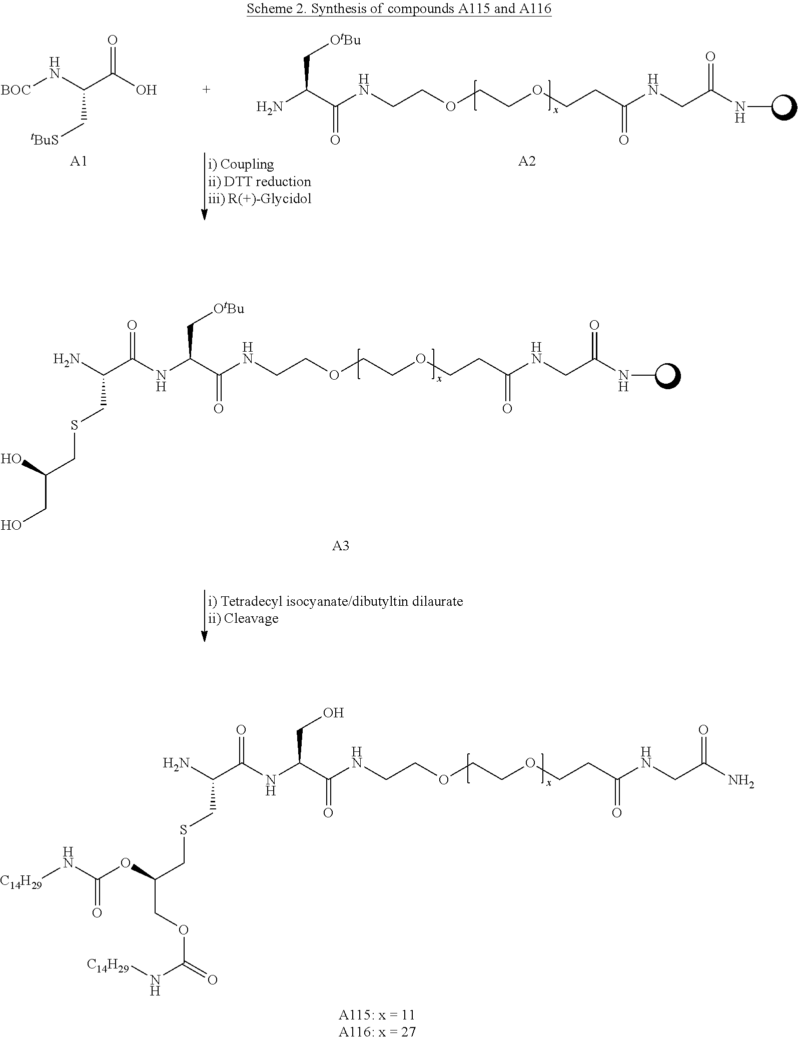

### Synthesis of Compounds A117 and A118

Compounds A117 (X═S(═O)) and A118 (X═S(═O)₂) were prepared following a similar synthetic routes as described above for compound A115, with the omission of ethylmethylsulfide scavenger, and optional omission of nitrogen sparging, from the carbamate formation step. Omitting the ethylmethylsulfide scavenger yielded a mixture of compounds A115, A117 and A118 which were separated and purified by HPLC.

Alternatively, sulfone or sulfoxide derivatives (eg A117 and A118) may be prepared by oxidation of the corresponding sulfide (eg A115) with an oxidant such as meta-chloroperoxybenzoic acid (MCPBA) or tert-butyl hydroperoxide (t-BuOOH) under appropriate conditions.

### Synthesis of Compounds A203 and A204

The synthesis of compounds A203 and A204 is depicted below in Scheme 3.

Fmoc-Gly was added as the first amino acid to the solid support, followed by coupling of Fmoc-NHCH₂CH₂O—(PEG)₁₁-CH₂CH₂COOH or Fmoc-NHCH₂CH₂O—(PEG)₂₇-CH₂CH₂COOH in 2-fold molar excess in presence of a two-fold excess of Hexafluorophosphate Benzotriazole Tetramethyl Uronium (HBTU), Hydroxybenzotriazole (HOBT) and 4-fold excess of diisopropylethylamine (DIPEA) in 2 ml of dimethylformamide (DMF) for 2 hrs. Fmoc-Ser(tBu)-OH is then coupled to provide intermediate A2, followed by the coupling of Boc-Cys(StBu) A1. The thiol-tert-butyl group on the cysteine residue was removed by incubating the peptide resin in 0.5M of dithiothreitol for 1 hr in DMF at RT. To 250 mg of Boc-Cys-Ser(tBu)-NHCH₂CH₂O—(PEG)₁₁-CH₂CH₂C(O)Gly resin or Boc-Cys-Ser(tBu) CH₂CH₂O—(PEG)₂₇-CH₂CH₂C(O)-Gly resin (0.25 mmole/g, 0.25 g=0.0625 mmole) saturated in DMF was added 250 μl of R-(+)-1,2-epoxy-butan-4-ol [(R)-2-(oxiran-2-yl)ethan-1-ol] (Mw=88.11, d=1.1, 250 μl=3.125 mmol equivalent to a 50 fold excess over the free sulfhydryl group present on the peptide resin) and 25 μl of diisopropylethylamine (DIPEA, Mw=129.2, d=0.74, 25 μl=0.14 mmol). The reaction mixture was left in a water bath at 50° C. for 2 hrs and then thoroughly washed with DMF to provide intermediate A3.

Palmitic acid (320 mg, 1.25 mmol), DIPCDI (225 uL, 1.5 mmol) and 4-dimethylaminopyridine (DMAP; 15.25 mg, 0.125 mmol) were dissolved in 2 mL of dichloromethane (DCM) then added to the resin-bound BOC-Dhc-peptide resin A3 (0.0625 mmol, 0.25 g) and shaken for 16 h at room temperature. The supernatant was removed by filtration and the solid support thoroughly washed with DCM and dimethylformamide (DMF) to remove any residue of urea before being subjected to the cleavage process as described below.

The solid support bearing the assembled lipopeptide was exposed to reagent B (93% TFA, 5% water and 2% triisopropylsilane) for 2 hours. To isolate the product, most of the TFA was removed and the residue is then dissolved in 50% acetonitrile and purified immediately using the purification protocol described below or the material was freeze-dried and stored for later purification.

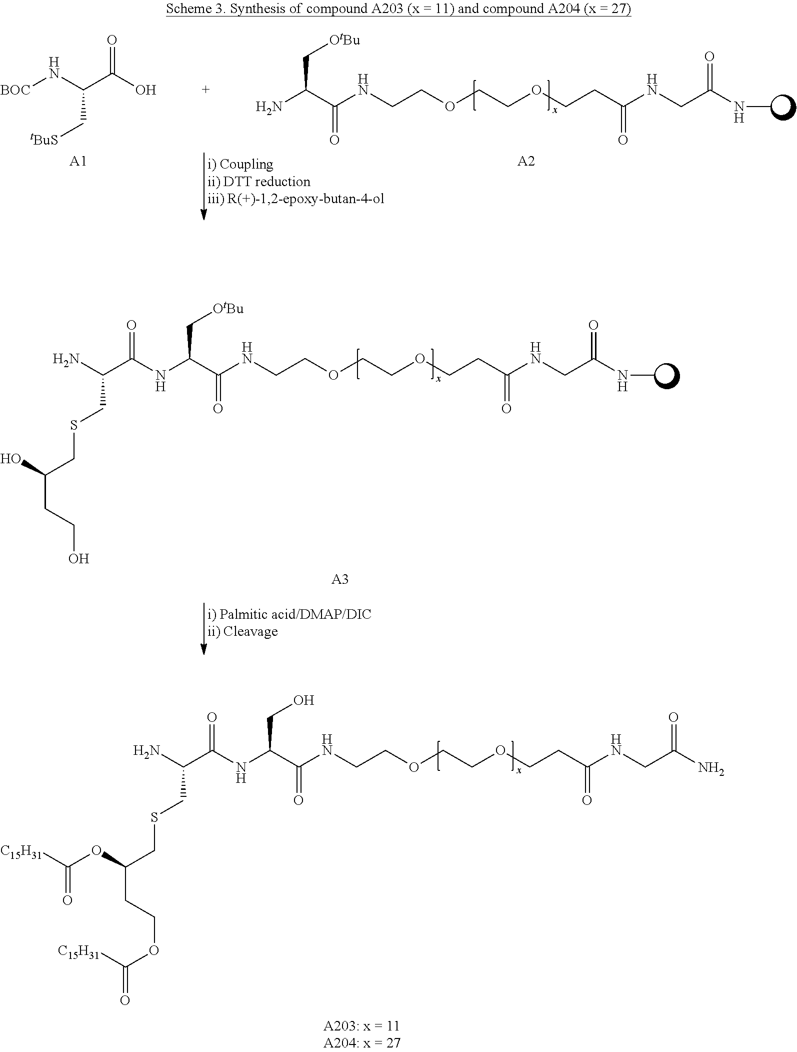

### Synthesis of Compound A215 and Compound A216

The synthesis of compounds A215 and A216 was carried out as depicted in Scheme 4. Intermediate A3 was prepared as described for compounds 3 and 4 above.

Then, to 250 mg of the peptide resin washed with toluene following glycidolation, were added 100 μl of ethylmethylsulfide (Mw=76.16, d=0.842, 100 μl=1.10 mmol) followed by 105 μl of tetradecyl isocyanate (MW=239, d=0.869, 105 μl=0.38 mmol, i.e. 3-fold excess over each of the hydroxyl groups present on the solid support) and finally 210 μl of dibutyltin dilaurate (Mw=631.6, d=1.053, 210 μl=0.35 mmol). The reaction mixture was sparged with nitrogen gas for approximately 5 min and mixed (Intelli—Mixer, RM-2, program F26 used) overnight at room temperature. The reaction mixture was transferred to a 50 ml tube and chloroform added to 50 ml. Following sonication for approximately 5 mins the white precipitate, formed during the reaction, dissolved. The solid support was washed with DMF and acetonitrile and the final product obtained following cleavage (as above) from the support was purified by HPLC.

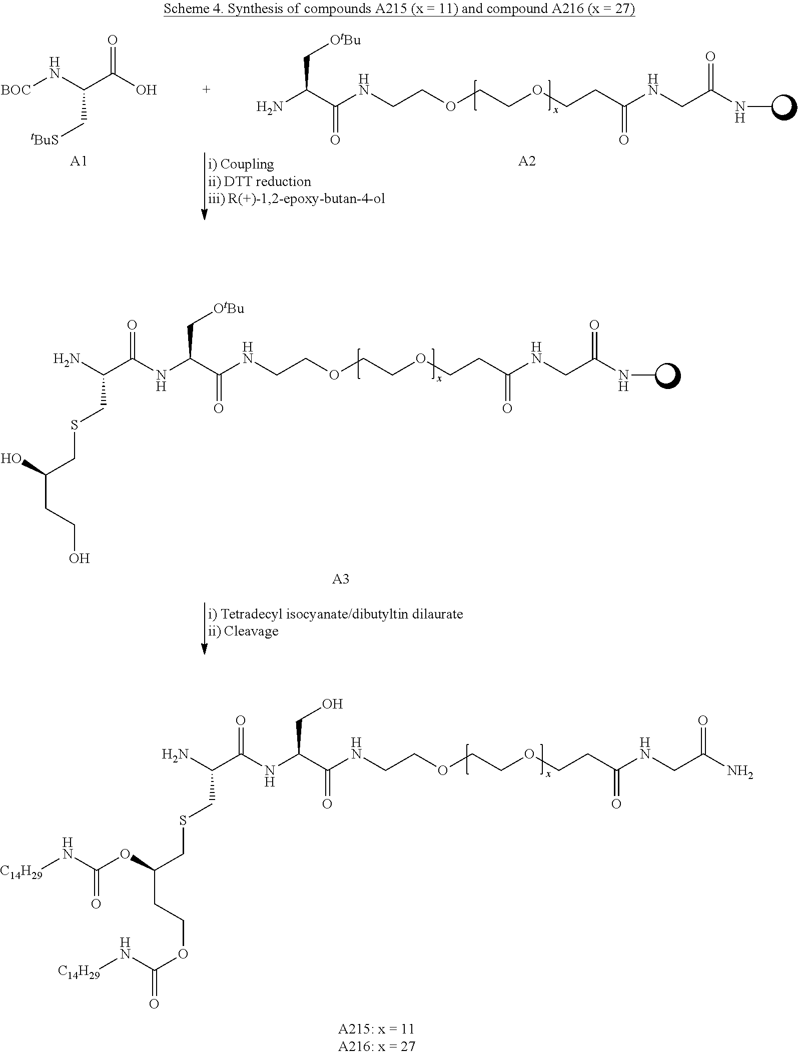

Synthesis of A220. Compound A220 was synthesized by standard Fmoc Solid Phase Peptide Synthesis, starting with Fmoc-RINK MBHA PS Resin. Removal of the Fmoc group after each coupling was achieved using 20% piperidine in DMF. Couplings of Fmoc-Gly-OH (2-fold excess), Fmoc-NH-PEG₂₈-CH₂CH₂COOH (1.4-fold excess), Fmoc-Ser(tBu)-OH (2-fold excess), and N-(Boc)-S—((R)-2,3-dihydroxybutyl)-L-cysteine (1.5-fold excess) were performed in DMF using equivalent excess of ethyl cyano(hydroxyimino)acetate (Oxyma Pure) and diisopropylcarbodiimide (DIC) as coupling agents. Myristyl Chloroformate coupling was performed using Myristyl Chloroformate (12 eq. vs. moles resin), DIEA (24 eq. vs. moles resin) in dry DCM for 18 hours at room temperature. This coupling was repeated three times (“recoupling”). The first recoupling was done using Myristyl Chloroformate (12 eq. vs. moles resin), NMM (24 eq. vs. moles resin) in dry DCM/THF (85/15) for 18 hours at room temperature. The second recoupling was done using Myristyl Chloroformate (6 eq. vs. moles resin), NMM (12 eq. vs. moles resin) in dry DCM/THF (85/15) for 41 hours at room temperature. Finally the third recoupling was performed using Myristyl Chloroformate (6 eq. vs. moles resin), NMM (12 eq. vs. moles resin) in dry DCM/THF/Toluene (85/15/5) for 21.5 hours at room temperature.

Cleavage of the peptide from the resin, removal of N-terminal Boc group, and serine side-chain deprotection were achieved by exposure of the resin to a solution of 93% trifluoroacetic acid (TFA), 5% H₂O, 3% triisopropylsilane (TIPS) for 1.5 hours. Following the cleavage reaction, the mixture was evaporated and the resulting residue was re-dissolved in 30% acetonitrile/water and lyophilized.

Synthesis of A224. Compound A224 was synthesized by standard Fmoc Solid Phase Peptide Synthesis, starting with Chlorotrityl Chloride Resin with an initial substitution of 1.6 meq/g. The first amino acid, Fmoc-Gly-OH, was loaded on the resin first, using a 0.5-fold molar excess of Fmoc-Gly-OH and DIEA (1.5-fold excess), followed by capping with DMF/MeOH/DIEA (80/10/10), and Fmoc deprotection, to obtain the dry loaded H-Gly-CT Resin with a final substitution of 0.67 meq/g. Removal of the Fmoc group after each coupling was achieved using 20% Piperidine in DMF. Coupling of Fmoc-NH-PEG₂₈-CH₂CH₂COOH (1.4 eq.) was performed using (7-Azabenzotriazol-1-yloxy)trispyrrolidinophosphonium hexafluorophosphate (PyAOp; 1.4 eq.), diisopropylethylamine (DIEA; 3.2 eq.) in DMF, whereas couplings of Fmoc-Ser(tBu)-OH (2 eq), and N-(Boc)-S—((R)-2,4-dihydroxybutyl)-L-cysteine (1.5 eq.) were performed in DMF using equivalent excess of Oxyma Pure and DIC as coupling agents. Palmitic Acid coupling was performed using palmitic acid (20 eq. vs. moles resin), DIC (20 eq.), DMAP (2 eq.) in DCM/THF (85/15) (v/v) for 24 hours at room temperature.

Cleavage of the peptide from the resin, removal of N-terminal Boc group, and serine side-chain deprotection were achieved by exposure of the resin to a solution of 93% TFA, 5% H2O, 3% TIPS for 1.5 hours. Following the cleavage reaction, the mixture was evaporated and the resulting residue was re-dissolved in 30% Acetonitrile/Water and lyophilized.

Purification and Characterisation

Purification and characterisation: Following cleavage from the solid support, each of the analogs were purified by reversed-phase HPLC according to either protocol A or B described below.

Protocol A: Reversed phase HPLC was conducted using an Agilent Zorbax 300SB-C3, 5 um column (9.4 mm×250 mm; Agilent Technology, Australia) installed in an Agilent HPLC 1260 Infinity system (Agilent Technologies, Santa Clara, Calif., USA) with the chromatogram developed using Buffer A (0.1% trifluoroacetic acid in water) and buffer B (0.1% trifluoroacetic acid in acetonitrile).

Protocol B: Reverse phase chromatography was conducted using a Novasep Axial Compression Column (5-cm diameter) loaded with cyano media (Daisogel SP-120-CN-P), with a gradient of Acetonitrile in [0.1% TFA/Water]. Following intermediate lyophilization, ion-exchange was performed on Dowex ion-exchange resin in order to obtain the peptide as the acetate salt.

Identification and purity determination of the target materials were carried out using an in-line HPLC-MS system using the following conditions:

Conditions A: HPLC column: Agilent Zorbax 300-SB C3 (150×0.5 mm; 5 μm) with the following gradient conditions: 0-5 min, 20% B: 5-32 min, 20% B-100% B: 32-40 min, 100% B-20% B. The flow rate was 20 μl/min. LC-MS: Agilent 1100 series capillary LC system in-line with an Agilent 1100 series LC/MSD ion-trap mass spectrometer. The mass spectrometer was operated with electrospray ionisation configured in the positive ion mode. Data analysis software from Agilent Technologies was used to de-convolute the charged ion series for identification of the peptide material and the material then characterised by LC-MS.

Conditions B: analytical reverse phase HPLC with a cyano column (Daiso Fine Chem, SP-120-3-CN-P, 150×4.6 mm, 3 μm, 120 Å). The peptide was also analyzed by ESI LC-MS in Positive Ion Mode, using a Finnigan LCQ Deca XPMax.

Compounds A107, A108, A115, A116, A203, A204, A215 and A216 prepared and purified as described above following protocol A and conditions A, compounds A220 and A224 were prepared and purified as described above following protocol B and conditions B, and were each found to be greater than 95% pure.

Peptide Quantitation

Quantitation of compounds A107, A108, A115, A116, A203, A204, A215 and A216 was carried out by in vacuo hydrolysis at 110° C. of samples in sealed glass vials in the presence of 6N HCl containing 0.1% phenol. Derivatisation of amino acids was then carried out using Waters AccQTag reagents according to the manufacturer's instructions followed by analysis on a Waters Acquity UPLC System (Waters Millipore) using an AccQTag ultra column (2.1 mm×100 mm; Waters Millipore). Quantitation of other compounds may be achieved by a similar protocol.

### 1.5—Synthesis of Sulfone and Sulfoxide Analogues of Compounds 15 and 16

Sulfone and sulfoxide derivatives of compounds 15 and 16 may be accessed by a similar synthetic routes as described above, with the omission of ethylmethylsulfide scavenger, and optional omission of nitrogen sparging, from the carbamate formation step. This reaction may yield a mixture of thiol, sulfone and sulfoxide derivatives, which may be separated and purified by HPLC.

Alternatively, sulfone or sulfoxide derivatives may be prepared by oxidation of the corresponding sulfide with an oxidant such as meta-chloroperoxybenzoic acid (MCPBA) or tert-butyl hydroperoxide (t-BuOOH) under appropriate conditions.

### Example 2—Activation of Human TLR2

### 2.1 In Vitro mTLR2 and hTLR2 Assay for Compounds 3, 4, 15 and 16

The potency of the compounds as activators of human and mouse TLR-2s is tested in an in vitro assay. The assay assesses NF-kB activation in the HEKBlue-mTLR-2 cell line. These cells have been stably transfected with mouse TLR-2 and express TLR-1 and TLR-6 endogenously at sufficient levels to allow for fully-functional TLR-1/2 and TLR-2/6 activation.

Toll-Like Receptor 2 (TLR2) stimulation is tested by assessing NF-kB activation in the HEKBlue-hTLR2 cell line. These cells have been stably transfected with human TLR2 and express TLR1 and TLR6 endogenously at a level sufficient to allow for fully-functional TLR1/2 and TLR2/6 activation. The activity of the test articles are tested on human TLR2 as potential agonists. The test articles are evaluated at seven concentrations and compared to control ligands. These steps are performed in triplicate.

NF-kB reporter gene assay protocol: HEK293T cells were cultured in 96-well plates at 4×10⁴ cells/well and transfected 24 h later with 100 ng of the NF-kB luciferase reporter gene [50 ng of TK-Renilla-luciferase expressing plasmid (Promega corporation, Madison, USA)] with or without 5 ng TLR2-expressing plasmid in the presence of 0.8 μl Fugene 6 (Roche Diagnostic). Compounds were added to the wells 24 h later at the concentrations indicated in the histograms. Cell lysates were prepared 5 h after stimulation using reporter lysis buffer (Promega Corporation, Madison, USA). Luciferase activities in the cell lysates were determined using a reagent kit (Promega Corporation, Madison, USA) and using a FLUOstar microplate reader (BMG Labtech, Ortenberg, Germany). The NF-kB-dependent firefly luciferase activity is normalised with NF-kB-independent renilla luciferase activity. The relative stimulation was calculated as the ratio of the stimulated to non-stimulated samples.

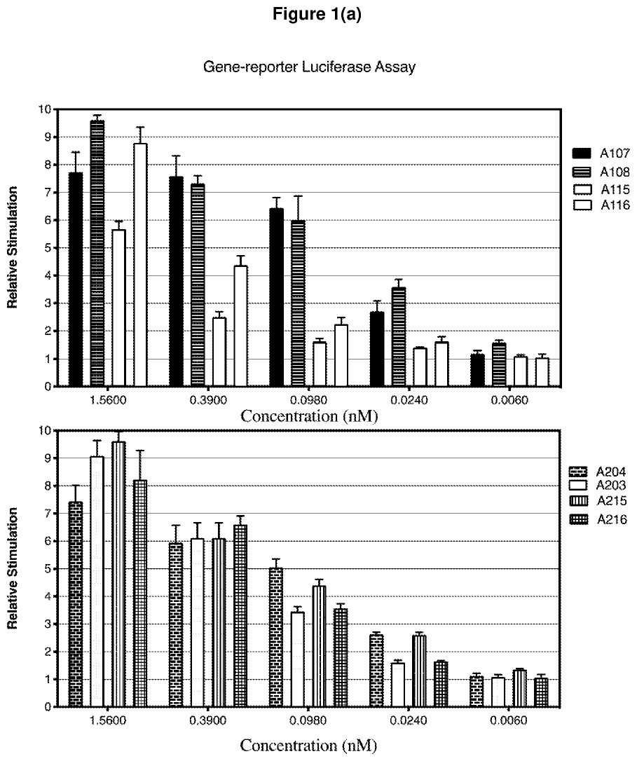

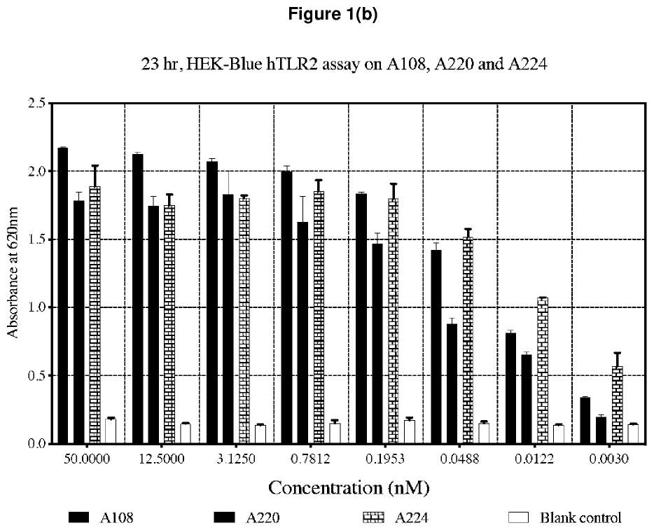

The results of this assay for compounds A107, A108, A115, A116, A203, A204, A215 and A216 are shown in FIG. 1(a). These data show that these compounds exhibit significant activity at TLR2.

### 2.2 In Vitro hTLR2 Assay for Compounds A108, A220 and A224

The potency of the compounds as activators of human TLR-2s is tested in an in vitro assay in HEK-BLUE-hTLR2 cells.

Culturing of HEK-BLUE-hTLR2 Cells

HEK-BLUE-hTLR2 cells are designed for studying the stimulation of human TLR2 (hTLR2) by monitoring the activation of NF-kB. HEK-BLUE-hTLR2 cells are obtained by co-transfection of the hTLR2 and SEAP (secreted embryonic alkaline phosphatase) reporter genes into HEK293 cells. Stimulation with a TLR2 ligand activates NF-kB which induces the production of SEAP.

HEK-BLUE-hTLR2 cells were purchased from InvivoGen (San Diego, Calif., USA). Cells were grown in DMEM supplemented with 10% FCS, 100 U/ml penicillin, 100 ug/ml streptomycin and 2 mM L-glutamine, 100 μg/mL Normocin in the presence of selection antibiotic purchased from InvivoGen and passaged when 70% confluence was reached per manufacturer's recommendation. Cells were dislodged and resuspended in test media as suggested by manufacturer for testing.

Testing of Compounds
i) A serial dilution of respective compounds were prepared in saline and added in 20 ml of each dilution in triplicates per well in a flat bottom 96-well plate and placed in the incubator while waiting for the cells.
ii) Remove HEK-BLUE-hTLR2 cells in a T-75 flask from incubator and discard the growth media.
iii) Gently rinse the cells with prewarmed 10 ml of PBS
iv) Add 5 ml of prewarmed PBS and place the cells in 37° C. for 2 mins and then detach the cells by gently pipetting up and down the PBS on the surface where the cells adhere.
v) Cells suspension at the density of 280,000 cells/ml is prepared in HEK-Blue™ Detection medium which is purchased from InvivoGen and prepared according to the manufacturer's instruction,
vi) Add immediately 180 ml of the cell suspension per well of the plate which contains the solution of the compounds. The plate is then returned to the incubator at 37° C. for 16 hr and was read at 620 nm by using an ELISA reader.

The results of this assay for compounds A108, A220 and A224 are shown in FIG. 1(b). These data show that these compounds exhibit significant activity at TLR2.

### Example 3—Coronavirus—Prophylaxis and Treatment

In December 2019 several pneumonia cases of unknown cause emerged in Wuhan, Hubei, China. Deep sequencing analysis from lower respiratory tract samples from patients indicated the cause to be a novel coronavirus. The causative agent of this novel Coronavirus disease (COVID-19) is the Coronavirus SARS-CoV-2. On 11 Mar. 2020, the WHO declared COVID-19 a pandemic.

Previously ferrets have been used as an animal model for SARS-CoV due to the presence of ACE2 (a receptor for SARS-CoV) used by the virus to gain entry to cells. Examination of the whole genome shows that SARS-CoV-2 has a ˜80% similarity to SARS-CoV and analysis has shown that both viruses use ACE2 as a receptor.

Currently, no therapeutics are being applied to the treatment of SARS-CoV-2. A series of novel, synthetic compounds with TLR2 agonist properties have been developed.

The purpose of this study is to assess the ability of compound A204 to protect ferrets against neutrophil-induced airway inflammation, a common cause of hospitalisation in human COVID-19 patients, through prophylactic treatment of ferrets with A204 before SARS-CoV-2 challenge, then sampling throughout the study to detect viral RNA.

Preparation of Treatment Doses

The 1 mg/ml solution of A204 was removed from 2-8° C. storage prior to dosage preparations. Doses were prepared to 100 μg or 20 μg, according to the following schedule:

Group 1; 100 μg, 1 ml (1 mg/ml solution) added to 9 ml sterile PBS, 4 and 1 days before challenge.

Group 2; 20 μg, 200 μl (1 mg/ml solution) added to 9.8 ml sterile PBS, 4 and 1 days before challenge.

Group 3; 20 μg, 200 μl (1 mg/ml solution) added to 9.8 ml sterile PBS, 4 days before challenge. 100 μg was also administered to Group 3 animals so it was prepared 1 day before challenge.

Challenge Item—SARS-Coronavirus-2

The challenge substance was SARS-CoV-2 virus, VERO/hSLAM cell passage 3, Animal challenge pool, identification number ASL401 (Titre: 2.4×10⁷ pfu/ml). The challenge substance was stored frozen at ≤−60° C. until required. The required dose for this study was 5.0×10⁶ pfu. Challenge substance dilutions were conducted on the day of challenge in Phosphate Buffered Saline

Experimental Animals

Species: Ferret

Health status: Conventional (Highgate Farm)

Source of supply: Highgate Farm

Number required: 24

Sex: Female

Weight: >550 g at time of treatment

Experimental Protocol

TABLE 1
Study Design

| | | | | | Cull | |
|---|---|---|---|---|---|---|
| | Number | Test/ | Treatment | | day 3 | Cull days* |
| | of | control | day | Challenge | (no. | (remaining |
| Group | ferrets | item | (s) | day | ferrets) | ferrets) |
| 1 | 6 | A204 | −4, −1 | 0 | 2 | 12-14 |
| | | (100 μg) | | | | |
| 2 | 6 | A204 | −4, −1 | 0 | 2 | 12-14 |
| | | (20 μg) | | | | |
| 3 | 6 | A204 | −4, −1 | 0 | 0 | 12-14 |
| | | (20 μg, | | | | |
| | | 100 μg) | | | | |
| 4 | 6 | Sterile | −4, −1 | 0 | 2 | 12-14 |
| | | PBS | | | | |

Following treatment with test or control item, EDTA blood samples and swabs were taken to detect viral RNA.

Pre-treatment blood samples, nasal washes and swabs were taken from all animals 4 days prior to challenge to enable baseline determinations to be made. Post challenge EDTA bloods, throat swabs and nasal wash were collected when animals were sedated. Under sedation, blood was taken from the cranial vena cava vessel. Nasal washes were performed using 2 ml PBS. Throat swabs will be taken into 1 ml viral transport medium.

Intranasal Administration of Compounds

All animals in group 1 were treated with 2 doses of 100 μg A204 by administering 1 ml intranasally. Group 2 received 2 doses of 20 μg A204 pre-challenge. Group 3 received 1 dose of 20 μg 4 days pre-challenge and 1 dose of either 100 μg 1 day pre-challenge. All animals in group 4 received 1 ml of sterile PBS intranasally.

Virus Challenge

Under sedation, inoculum was administered using a 1 ml syringe with feeding tube attached. The total volume of 1 ml of inoculum material was administered and distributed evenly between both nares, resulting in a challenge dose of 5.0E+06 pfu of virus. This procedure was performed slowly, ensuring each droplet has gone into the nasal cavity before releasing another droplet.

Results

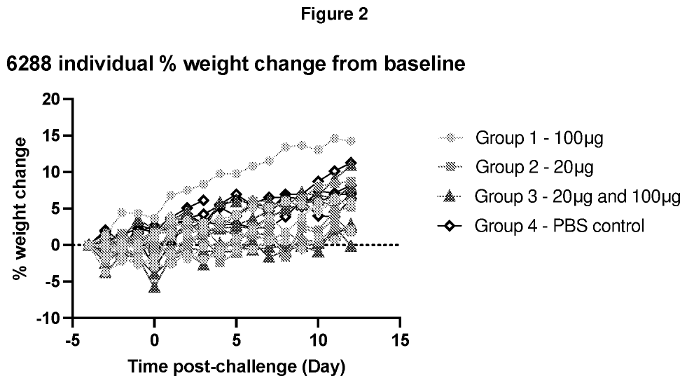

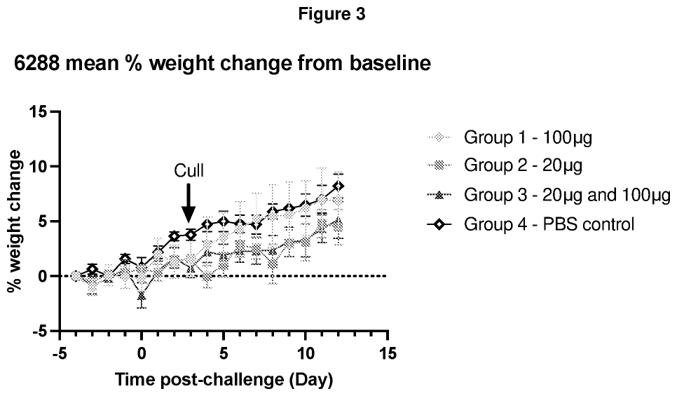

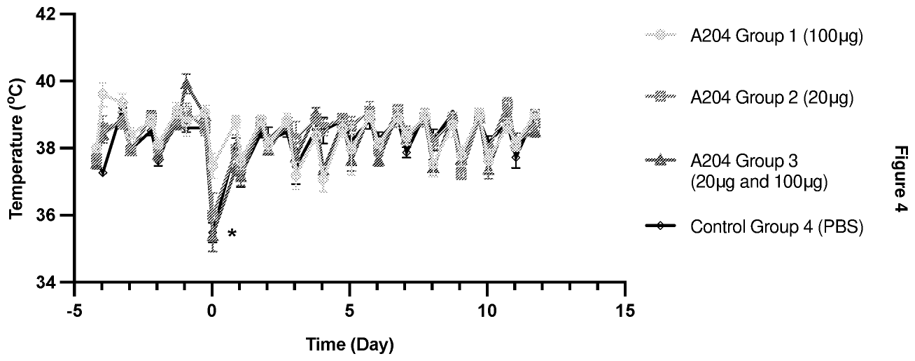

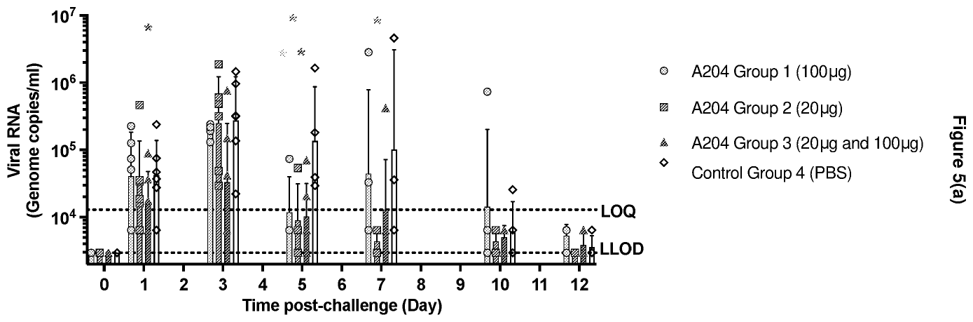

The results in FIGS. 2 and 3 show that A204 did not cause a reduction in body weight of the animals. Further, there was no change in mean temperature (FIG. 4).

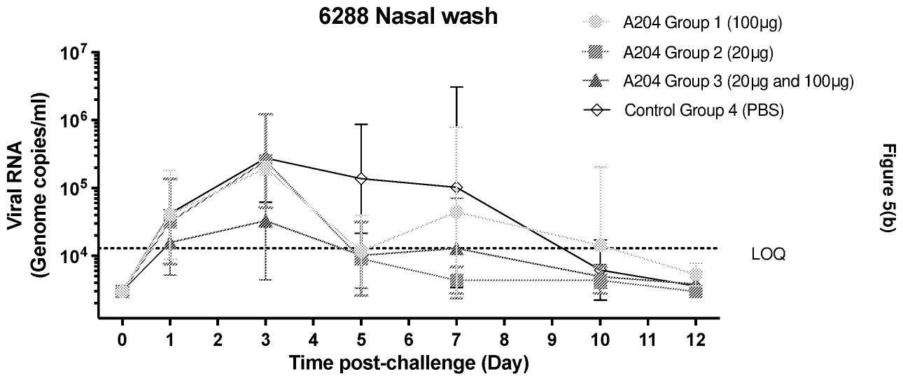

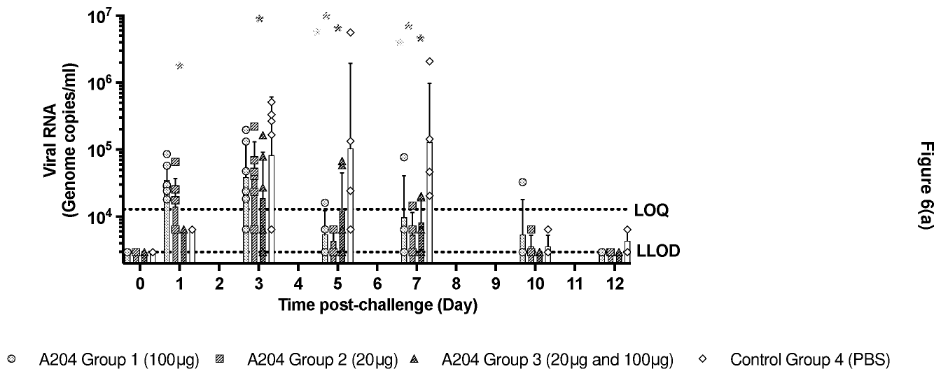

A204 significantly reduced viral load in the nasal cavity and in the throat in Groups 1, 2 and 3 animals (FIGS. 5 and 6).

A 2-way ANOVA was performed with a Dunnett's test. This compares the means of individual logged data at each time point for each treatment group relative to the control group. This is a sensitive and relevant test to use on looking for significance between groups.

The following results showed significance between groups at days 3, 5 and 7 based on the PCR data from the nasal wash:

| | Predicted (LS) | | | Adjusted |
|---|---|---|---|---|
| Dunnett's multiple comparisons test | mean diff. | 95.00% CI of diff. | Significant? | P Value |
| Day 3 | | | | |
| Group 4 - PBS control vs. Group 1 - 100 ug | 0.1444 | −0.6851 to 0.9739 | No | 0.9547 |
| Group 4 - PBS control vs. Group 2 - 20 ug | 0.04063 | −0.7889 to 0.8701 | No | 0.9988 |
| Group 4 - PBS control vs. Group 3 - 20 ug and 100 ug | 0.9236 | 0.09409 to 1.753 | Yes | 0.0249 |
| Day 5 | | | | |
| Group 4 - PBS control vs. Group 1 - 100 ug | 1.063 | 0.04978 to 2.076 | Yes | 0.0373 |
| Group 4 - PBS control vs. Group 2 - 20 ug | 1.271 | 0.2581 to 2.284 | Yes | 0.0098 |
| Group 4 - PBS control vs. Group 3 - 20 ug and 100 ug | 1.188 | 0.2632 to 2.113 | Yes | 0.0079 |
| Day 7 | | | | |
| Group 4 - PBS control vs. Group 1 - 100 ug | 0.3619 | −0.7247 to 1.449 | No | 0.7475 |
| Group 4 - PBS control vs. Group 2 - 20 ug | 1.55 | 0.4630 to 2.636 | Yes | 0.0029 |
| Group 4 - PBS control vs. Group 3 - 20 ug and 100 ug | 0.8985 | −0.1076 to 1.904 | No | 0.0897 |

The 2-way ANOVA results using a Dunnett's test showed significance between groups at days 1, 3, 5 and 7 from the PCR date from the throat swab:

| | Predicted (LS) | | | Adjusted |
|---|---|---|---|---|
| Dunnett's multiple comparisons test | mean diff. | 95.00% CI of diff. | Significant? | P Value |
| Day 1 | | | | |
| Group 4 - PBS control vs. Group 1 - 100 ug | −0.7352 | −1.407 to −0.06348 | Yes | 0.0281 |
| Group 4 - PBS control vs. Group 2 - 20 ug | −0.3409 | −1.013 to 0.3309 | No | 0.4834 |
| Group 4 - PBS control vs. Group 3 - 20 ug and 100 ug | 0 | −0.6718 to 0.6718 | No | >0.9999 |
| Day 3 | | | | |
| Group 4 - PBS control vs. Group 1 - 100 ug | 0.324 | −0.3477 to 0.9958 | No | 0.5232 |
| Group 4 - PBS control vs. Group 2 - 20 ug | 0.3091 | −0.3627 to 0.9808 | No | 0.5596 |
| Group 4 - PBS control vs. Group 3 - 20 ug and 100 ug | 0.7038 | 0.03209 to 1.376 | Yes | 0.0376 |
| Day 5 | | | | |
| Group 4 - PBS control vs. Group 1 - 100 ug | 1.457 | 0.6369 to 2.278 | Yes | 0.0001 |
| Group 4 - PBS control vs. Group 2 - 20 ug | 1.558 | 0.7372 to 2.378 | Yes | <0.0001 |
| Group 4 - PBS control vs. Group 3 - 20 ug and 100 ug | 0.9944 | 0.2455 to 1.743 | Yes | 0.0058 |
| Day 7 | | | | |
| Group 4 - PBS control vs. Group 1 - 100 ug | 1.211 | 0.3907 to 2.032 | Yes | 0.0019 |
| Group 4 - PBS control vs. Group 2 - 20 ug | 1.565 | 0.7447 to 2.386 | Yes | <0.0001 |
| Group 4 - PBS control vs. Group 3 - 20 ug and 100 ug | 1.259 | 0.5103 to 2.008 | Yes | 0.0003 |

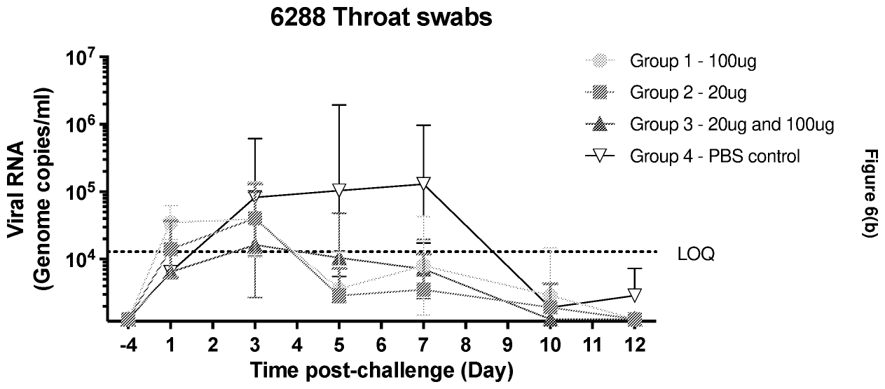

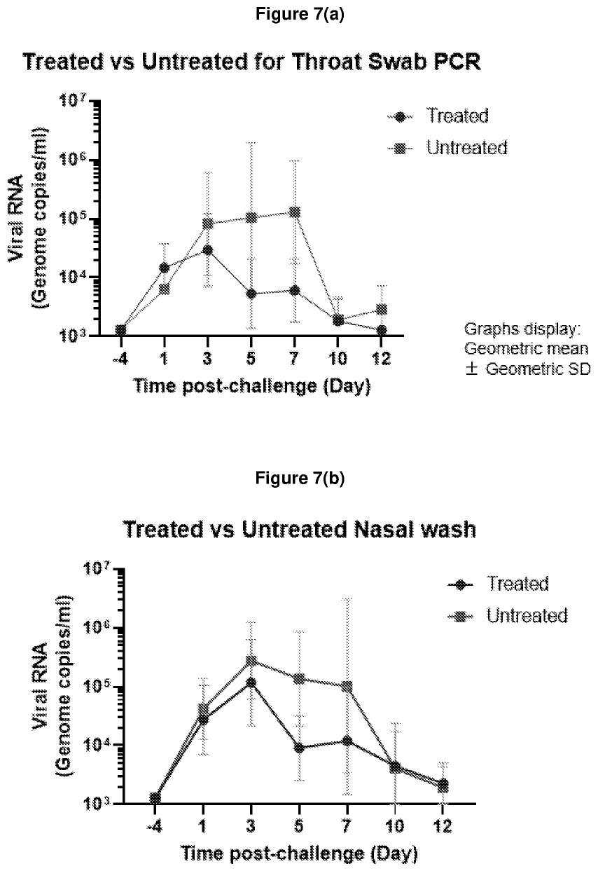

FIG. 7 combines the individual logged data for all treatment groups, groups 1 to 3, and compares that against the untreated group, group 4. A two-way ANOVA (Sidak's multiple comparison test) showed there were significant differences at day 5 and day 7, p=<0.0001, for the throat swab (FIG. 6(a)) and day 5, p=0.0073, for nasal wash (FIG. 6(b)).

### Example 4—Upper Respiratory Control of Coronavirus Infection in Mice by Prophylactic TLR2 Agonist Treatment to the Respiratory Tract

The present study was performed to determine an effect on endemic coronavirus strain replication in the upper respiratory tract (URT), or the ability to prime innate immune responses to coronavirus infection (endemic nor pandemic strains alike).

Objectives

To demonstrate the efficacy (dose/regimen) of TLR2 agonist-based nasal treatment during URT coronavirus infection in vivo. The TLR2-agonist used is A101 a different TLR2-agonist to that tested in Example 3 above.

TLR2 Agonists and Controls

1 nmol of A101 was diluted per 10 uL saline per mouse (141.48 μg/mL) and administered to lightly anaesthetised mice. Naïve/placebo controls (saline) were included for comparisons in all experiments.

Experimental Animals.

Female 6-8 week old BALB/c mice were used for all studies. Each treatment group contained 8 mice. After treatment or challenge procedures, mice were monitored daily for animal welfare, and behavioural or physical changes as stipulated in animal ethics.

Mouse Coronavirus Infection with Coronavirus OC43

Mice were infected intranasally (i.n.) with 10 μL containing 2.5×10⁶ TCID₅₀ of OC43 whilst under light isoflurane anaesthesia. Nasal turbinates, trachea and lung were collected for RNA extraction to assess viral load. Cytokines in nasal lavage were assessed as well as viral RNA. Bronchoalveolar lavage (BAL) was performed to enumerate inflammatory cell infiltrate in the lower airways.

Coronavirus OC43 (CoV-OC43) is a member of the species Betacoronavirus 1.

Study Protocol

Mice were dosed intranasally (i.n.) to the URT with 10 μL containing 1 nmol of A101 (or saline) day −7 (d-7/−168 hours post infection) and day −3 (d-3/−72 hours post infection) as depicted in the table below. Following treatment mice were infected i.n. with OC43. Viral loads (RNA) were determined in the nasal turbinates, lung and trachea tissue as well as nasal wash. Nasal- and bronchoalveolar-lavage (BAL) were performed to enumerate inflammatory cell infiltrates in the lower airways. Type III IFN and ISG expression was determined in the nasal mucosa.

| Total Mice | | Administration protocol | Infection | |
|---|---|---|---|---|
| 8/gp | Treatment groups | (10 μL intranasal) | Day 0 | Time of harvest |
| N = 8 per | 1) Saline d −7, d −3/ | Saline day −7, −3 | OC43 | Harvest day 0 (2 hr post-infection), |
| treatment | OC43 | | | day 1, 3, 5. Analysis of viral titres |
| group | 2) A101 d −7, d −3/ | 1 nmol A101 day −7, −3 | OC43 | in nasal, trachea and lung tissue by |
| 4 time | OC43 −1 nmol | | | qPCR. Immune gene and protein |
| points | 3) Untreated | | OC43 | expression in nasal turbinates, lung |
| | | | | tissue, nasal- and bronchoalveolar- |
| | | | | lavage |

Results

URT a 101 Treatment Promoted Sustained Lymphocyte Recruitment in BAL Following URT OC43 Infection

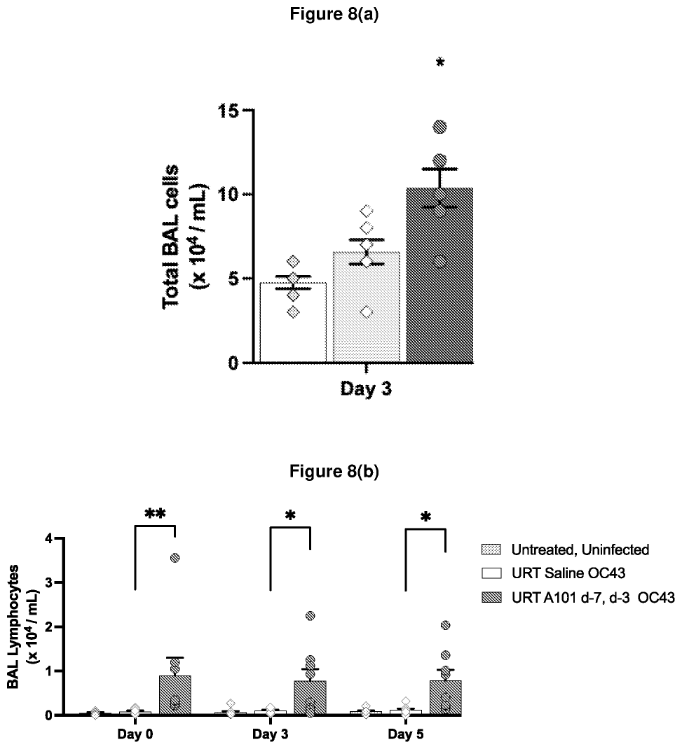

Mice were dosed with A101 and infected with 0043 as detailed above. Bronchoalveolar lavage (BAL) was performed at each harvest to enumerate immune cell infiltrate in the lower airways. Two treatments of A101 administered to the URT on day −7 and day −3 increased total BAIL leukocytes 3 days post URT 0043 infection (FIG. 8A). Furthermore, this dose protocol resulted in increased numbers of BAIL lymphocytes on day 0 (2-hours post infection) as well as day 3 and day 5 post infection (FIG. 8B).

URT a 101 Treatment Enhances Antiviral IFN-A and ISG Gene Expression Following OC43 Infection

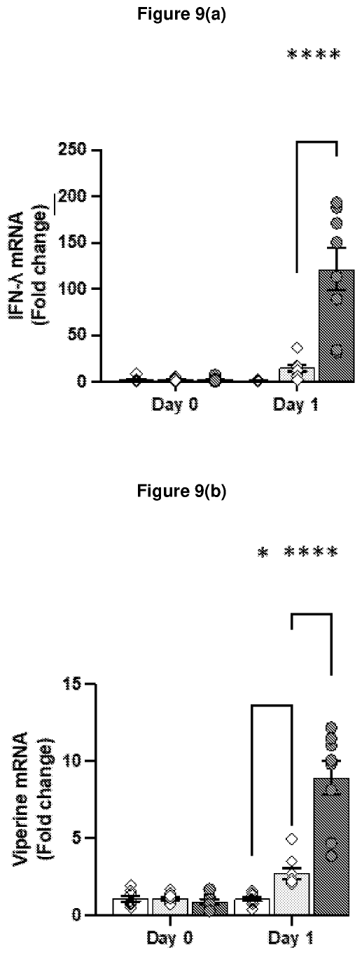

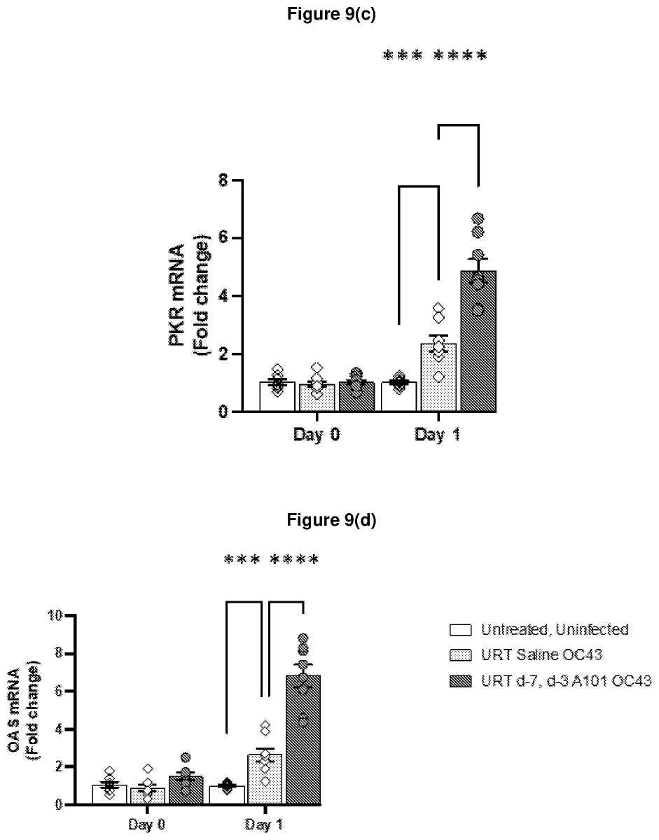

Antiviral gene expression of IFN-A, and IFN-response genes such as Viperin, PKR and OAS were assessed by qPCR in nasal turbinates. URT OC43 infection resulted in a small but statistically significant increase in IFN-A expression at day 1 post infection (FIG. 9A). OC43 infection also resulted in increased expression of viperin (FIG. 9B) as well as PKR (FIG. 9C) and OAS (FIG. 9D) expression on day 1 post infection. A101 treatment enhanced the antiviral response by dramatically increasing IFN-A, Viperin, PKR and OAS gene expression on day 1 post-infection (FIG. 9A-D).

OC43 Viral RNA in Nasal Wash Reduced by d-7 d-3 A101 Treatment

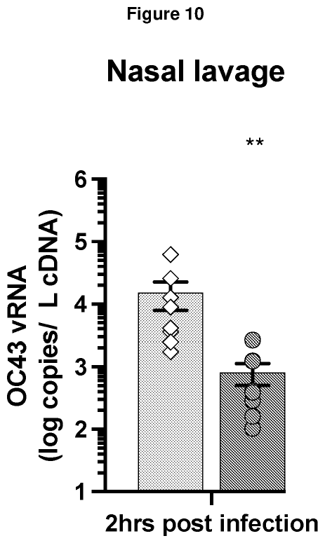

Viral load was assessed by qPCR in nasal wash. URT OC43 infection resulted in detectable virus in all upper respiratory tract samples (FIG. 10). An A101 dose on day −7 and a second dose of A101 on day −3 resulted in significantly less viral RNA in nasal wash 2 hours post infection (FIG. 10).

### Example 5—A204 Efficacy Against SARS-CoV-2 in the Hamster Model—Prophylaxis

To demonstrate the efficacy (dose/regimen) of TLR2 agonist-based nasal treatment during TRT SARS-CoV-2 in vivo in hamster model, six hamsters were mock treated with 100 ul PBS 24 hours prior to infection. Animals were infected with 10⁴ PFU SARS-CoV-2 in 100 ul, and weights were taken daily in addition to animal welfare scoring. Animals from all cohorts were throat swabbed to assess viral loads at 2 dpi and culled at 7 dpi.

Nasal tissue and lung tissue were harvested and utilized for RNA extraction and viral enumeration via RT-qPCR.

Results

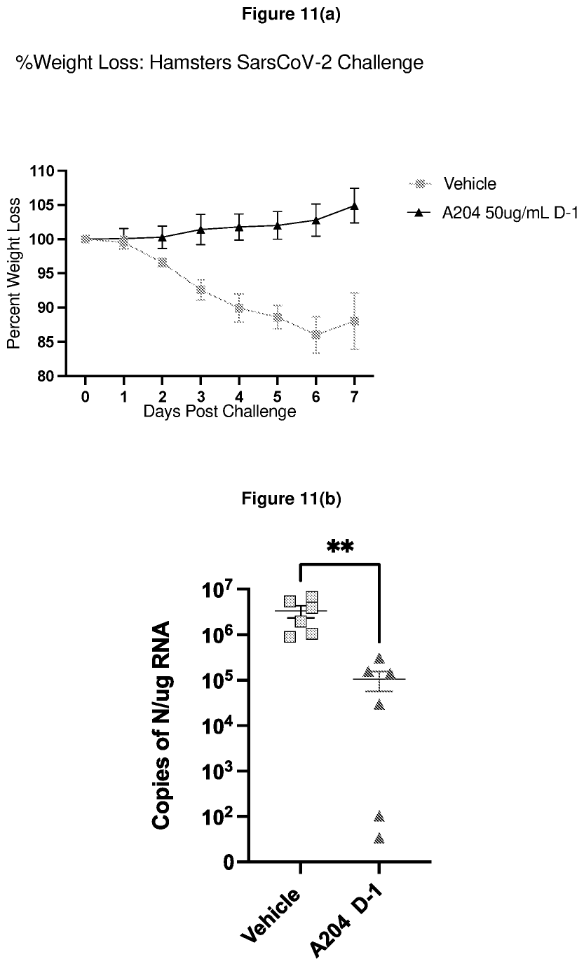

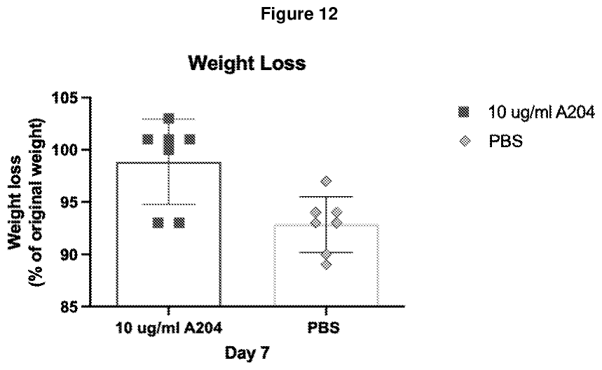

Animals pre-treated with A204 lost no weight during the course of the infection and instead steadily gained weight, indicating that pretreatment with 50 ug/ml conferred protection to the symptoms of SARS-CoV-2 infection (FIG. 11A).

Swabs were taken at day 2 post infection, total RNA extracted using TRizol reagent and RT-qPCR carried out to quantify viral load. At 2 days post infection the animals exhibited 32-fold lower levels of viral RNA compared to the mock treated animals (FIG. 11B).

At 7 dpi lung viral loads were found to be slightly lower in treated animals compared to the mock treated animals (data not shown).

### Example 6—A204 Efficacy Against SARS-CoV-2 in the Hamster Model—Treatment

Additionally, the inventors sought to investigate the effects of treatment with A204 shortly after SARS-CoV-2 infection. An experiment was undertaken to investigate the potential protective effect of treatment of hamsters with doses of A204 post infection with SARS-CoV-2. Groups (n=10) were infected with 10⁴ PFU SARS-CoV-2 and then treated 8 hours post infection (hpi) with 10 ug/ml A204 in 100 ul.

Results

Animals were infected with 10⁴ PFU SARS-CoV-2 and treated 8 hours post infection with 1 ug/100 ul (10 ug/ml) A204 or PBS. The group treated with 10 ug/ml recovered weight loss more rapidly than PBS treated and by 7 dpi were essentially back to original weight whereas PBS treated were not.

The experimental data described herein show that various TLR2 agonists, particularly agonists of a heterodimer of TLR2 and TLR6, can both prevent coronavirus infection and treat coronavirus infection, particularly where the coronavirus is SARS-CoV-2.

1. A method for reducing a coronavirus infection in a subject, the method comprising administering to the subject a therapeutically effective amount of a TLR2 agonist, thereby reducing the coronavirus infection in the subject.

2. The method of claim 1, wherein the TLR2 agonist is an agonist of a heterodimer of TLR2 and TLR6.

3. The method of claim 1 or 2, wherein the TLR2 agonist is a compound of formula (I):
A-Y—B (I)
wherein A comprises or consists of a moiety selected from A1 and A2: 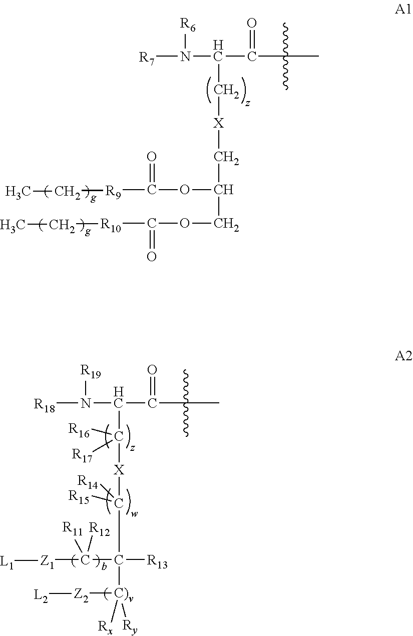
wherein
each z is independently selected from 1 or 2;
each X is independently selected from —S—, —S(═O)— and —S(═O)₂—;
in moiety A1:
each g is independently 10, 11, 12, 13, 14, 15, 16, 17 or 18;
R₆ and R₇ are independently selected from the group consisting of H, a straight or branched C₁-C₄ alkyl, and —C(═O)CH₃;
R₉ and R₁₀ are independently selected from the group consisting of —NH—, —O— or a single bond; and
in moiety A2:
b and w are each independently an integer from 0 to 7 and v is an integer from 0 to 5, such as from 2 to 5, provided that:
the sum of b, v, and w is at least 3; and
the sum of b and w is from 0 to 7;
Z₁ and Z₂ are each independently selected from the group consisting of —O—, —NR—, —S—, S(═O), —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A₂;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and La is optionally substituted;
Y is 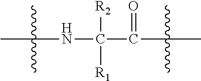
wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
and
B comprises or consists of Polyethylene Glycol (PEG),
or a pharmaceutically acceptable salt, solvate or prodrug thereof.

4. The method of claim 1 or 2, wherein the TLR2 agonist is a compound comprising moiety A selected from A1′ and A2 and PEG, wherein the moiety A and PEG are linked by a glycine, serine, homoserine, threonine, phosphoserine, asparagine or glutamine residue, or an ester of a glutamine residue,
wherein 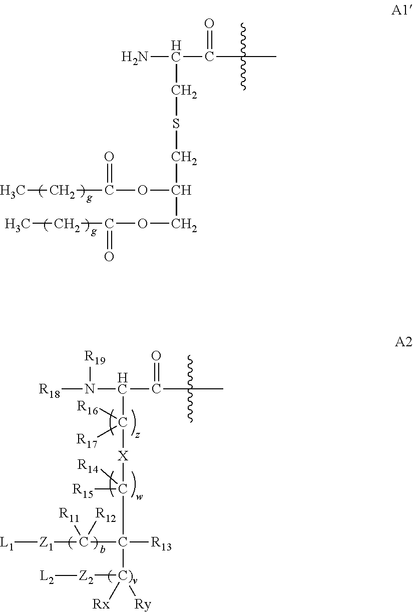
wherein
z is independently selected from 1 or 2;
X is independently selected from —S—, —S(═O)— and —S(═O)₂—;
each g is independently 10, 11, 12, 13, 14, 15, 16, 17 or 18;
b and w are each independently an integer from 0 to 7 and v is an integer from 0 to 5, such as from 2 to 5, provided that:
the sum of b, v, and w is at least 3; and
the sum of b and w is from 0 to 7;
Z₁ and Z₂ are each independently selected from the group consisting of —O—, —NR—, —S—, S(═O), —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A₂;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;
or a pharmaceutically acceptable salt, solvate or prodrug thereof

5. The method of claim 1 or 2, wherein the TLR2 agonist is a compound comprising or consisting of partial structure A1Y′ or A2Y′: 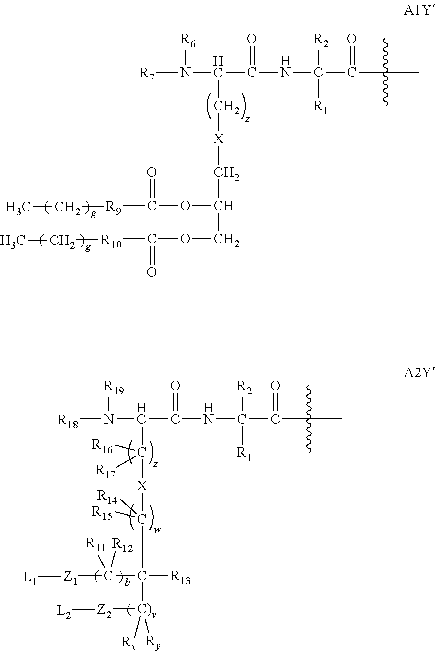
wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2;
X is selected from —S—, —S(═O)— and —S(═O)₂—;
b and w are each independently an integer from 0 to 7 and v is an integer from 0 to 5, provided that:
the sum of b, v, and w is at least 3; and
the sum of b and w is from 0 to 7;
Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A2;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted; and
A1Y′ or A2Y′ is covalently linked to polyethylene glycol (PEG),
or a pharmaceutically acceptable salt, solvate or prodrug thereof.

6. The method of any one of claims 3-5, wherein the PEG is a substituted PEG according to the following formula: 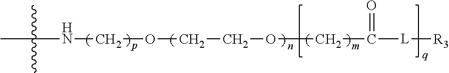
wherein
n is 3 to 100;
m is 1, 2, 3 or 4;
p is 2, 3 or 4;
q is null or 1;
R₃ is H, —NH₂ or —OH, wherein when q is null, R₃ is H and when q is 1, R₃ is —NH₂ or —OH;
L is null or consists of 1 to 10 units, wherein each unit is a natural alpha amino acid or derived from a natural alpha amino acid, and has the formula: 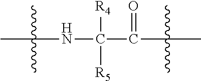
wherein R₄ is H; and
R₅ is the side chain, or second hydrogen of the amino acid.

7. The method of claim 1 or 2, wherein the TLR2 agonist is a compound of formula (VI): 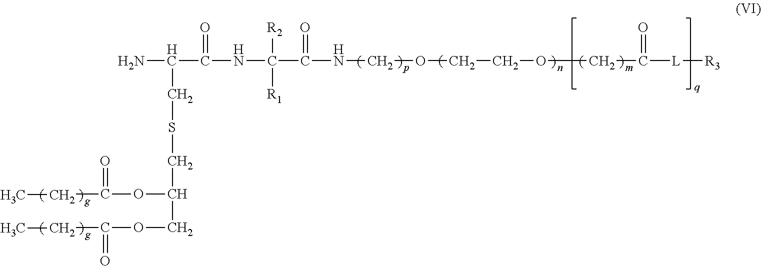
wherein
n is 3 to 100;
m is 1, 2, 3 or 4;
each g is independently 10, 11, 12, 13, 14, 15, 16, 17 or 18;
p is 2, 3 or 4;
q is null or 1;
R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula: 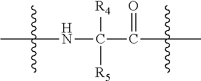
wherein R₄ is H; and
R₅ is the side chain, or second hydrogen of the amino acid
or a pharmaceutically acceptable salt, solvate or prodrug thereof.

8. The method of claim 1 or 2, wherein the TLR2 agonist is a compound of formula (VII): 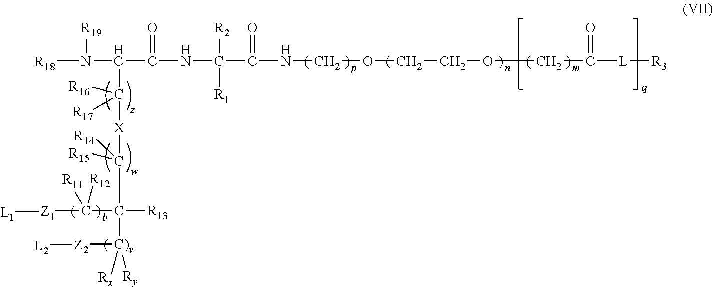
wherein
n is 3 to 100;
m is 1, 2, 3 or 4;
p is 2, 3 or 4;
q is null or 1;
R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and CH₂CH₂C(═O)OR₈, wherein any one of the alkyl hydrogens can be replaced with a halogen;
R₈ is selected from the group consisting of H and a straight or branched C₁-C₆ alkyl;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula: 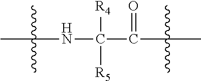
wherein R₄ is H; and
R₅ is the side chain, or second hydrogen of the amino acid;
b and w are each independently an integer from 0 to 7 and v is an integer from 0 to 5, provided that:
the sum of b, v, and w is at least 3; and
the sum of b and w is from 0 to 7;
z is 1 or 2;
X is selected from —S—, —S(═O)— and —S(═O)₂—;
Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A2;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;
or a pharmaceutically acceptable salt, solvate or prodrug thereof.

9. The method of claim 1 or 2, wherein the TLR2 agonist is a compound of formula (VIII):
A-Y—NH—(CH₂)ₚ—O—(CH₂—CH₂—O)ₙ—[(CH₂)ₘ—CO-L-]_{q}R₃ (VII)
wherein
A comprises or consists of a moiety selected from A1 and A2: 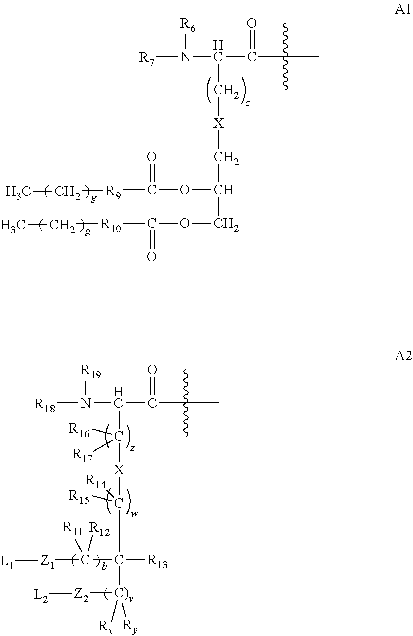
wherein
each z is independently selected from 1 or 2;
each X is independently selected from —S—, —S(═O)— and —S(═O)₂—;
in moiety A1:
each g is independently 10, 11, 12, 13, 14, 15, 16, 17 or 18;
R₆ and R₇ are independently selected from the group consisting of H, a straight or branched C₁-C₄ alkyl, and —C(═O)CH₃;
R₉ and R₁₀ are independently selected from the group consisting of —NH—, —O— or a single bond; and
in moiety A2:
b and w are each independently an integer from 0 to 7 and v is an integer from 0 to 5, such as from 2 to 5, provided that:
the sum of b, v, and w is at least 3; and
the sum of b and w is from 0 to 7;
Z₁ and Z₂ are each independently selected from the group consisting of —O—, —NR—, —S—, S(═O), —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A₂;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;
Y is 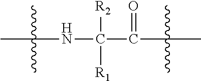
wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2;
X is selected from —S—, —S(═O)— and —S(═O)₂—;
n is 3 to 100;
m is 1, 2, 3 or 4;
each g is independently 10, 11, 12, 13, 14, 15, 16, 17 or 18;
p is 2, 3 or 4;
q is null or 1;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula: 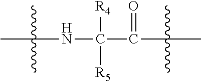
wherein R₄ is H; and
R₅ is the side chain, or second hydrogen of the amino acid,
or a pharmaceutically acceptable salt, solvate or prodrug thereof.

10. The method of any one of claims 6-9, or a pharmaceutically acceptable salt, solvate or prodrug thereof, wherein q is 1.

11. The method of any one of claims 6-10, or a pharmaceutically acceptable salt, solvate or prodrug thereof, wherein n is from 10 to 14.

12. The method of claim 11, or a pharmaceutically acceptable salt, solvate or prodrug thereof, wherein n is 11.

13. The method of any one of claims 6-10, or a pharmaceutically acceptable salt, solvate or prodrug thereof, wherein n is from 24 to 30.

14. The method of claim 13, or a pharmaceutically acceptable salt, solvate or prodrug thereof, wherein n is 27.

15. The method of any one of claims 6-14, or a pharmaceutically acceptable salt, solvate or prodrug thereof, wherein m is from 1 to 3.

16. The method of claim 15, or a pharmaceutically acceptable salt, solvate or prodrug thereof, wherein m is 2.

17. The method of any one of claims 3-16, or a pharmaceutically acceptable salt, solvate or prodrug thereof, wherein v is 2 to 5.

18. The method of any one of claims 3-17, or a pharmaceutically acceptable salt, solvate or prodrug thereof, wherein Rₓ, R_{y}, R₁₁, R₁₂, R₁₃, R₁₄, R₁₅, R₁₆, and R₁₇ are H.

19. The method of any one of claims 3-18, or a pharmaceutically acceptable salt, solvate or prodrug thereof, wherein Z₁ and Z₂ are the same and selected from the group consisting of —O—, —NR—, —S—, S(═O), S(═O)₂—, —C(═O)O—, —OC(═O)—, —C(═O)NR—, —NRC(═O)—, —C(═O)S—, —SC(═O)—, OC(═O)O—, NRC(═O)O—, —OC(═O)NR—, and —NRC(═O)NR—.

20. The method of any one of claims 3-19, or a pharmaceutically acceptable salt, solvate or prodrug thereof, wherein w is an integer from 1-7.

21. The method of any one of claims 3-20, or a pharmaceutically acceptable salt, solvate or prodrug thereof, wherein b is 0.

22. The method of any one of claims 3-21, wherein X is S.

23. The method of any one of the preceding claims, wherein the TLR2 agonist is selected from any one of compounds 001-010, A101-A114 and A201-A232, or a combination thereof.

24. A method of treating and/or preventing a disease associated with a coronavirus, comprising raising an innate immune response in a subject by administering an effective amount of a compound defined in any one of claims 1-23, or a pharmaceutically acceptable salt, solvate or prodrug thereof to the subject in need thereof, thereby treating and/or preventing a disease associated with a coronavirus.

25. A method of treating and/or preventing a disease caused by a coronavirus, comprising administering to a subject in need thereof an effective amount of a compound of any one of claims 1-23, or a pharmaceutically acceptable salt, solvate or prodrug thereof, thereby treating and/or preventing a disease caused by a coronavirus.

26. A method of treating and/or preventing a respiratory disease or condition associated with a coronavirus infection, comprising administering to a subject in need thereof a compound of any one of claims 1-23, or a pharmaceutically acceptable salt, solvate or prodrug thereof, thereby treating and/or preventing a respiratory disease or condition associated with a coronavirus infection.

27. A method of treating and/or preventing a coronavirus infection, comprising administering to a subject in need thereof a compound of any one of claims 1-23, or a pharmaceutically acceptable salt, solvate or prodrug thereof, thereby treating and/or preventing a coronavirus infection.

28. A method for reducing airway inflammation associated with or caused by a coronavirus infection, comprising administering to a subject in need thereof a compound of any one of claims 1-23, or a pharmaceutically acceptable salt, solvate or prodrug thereof, thereby reducing airway inflammation associated with or caused by a coronavirus infection.

29. A method of claim 27, wherein the method further comprises the step of identifying a subject having a coronavirus infection.

30. A method of improving the ability of a subject to control a respiratory disease or condition during a coronavirus infection, the method comprising administering to a subject in need thereof a compound of any one of claims 1-23, or a pharmaceutically acceptable salt, solvate or prodrug thereof, thereby improving the ability of a subject to control a respiratory disease or condition during a coronavirus infection.

31. Use of a compound of any one of claims 1-23 or a pharmaceutically acceptable salt, solvate or prodrug thereof in the preparation of a medicament for raising an innate immune response in a subject having a coronavirus infection.

32. Use of a compound of any one of claims 1-23 or a pharmaceutically acceptable salt, solvate or prodrug thereof in the preparation of a medicament for treating and/or preventing a disease caused by coronavirus.

33. Use of a compound of any one of claims 1-23 or a pharmaceutically acceptable salt, solvate or prodrug thereof in the preparation of a medicament for treating and/or preventing a respiratory disease or condition associated with a coronavirus infection in a subject.

34. Use of a compound of any one of claims 1-23 or a pharmaceutically acceptable salt, solvate or prodrug thereof in the preparation of a medicament for treating and/or preventing a coronavirus infection in a subject.

35. Use of a compound of any one of claims 1-23 or a pharmaceutically acceptable salt, solvate or prodrug thereof in the preparation of a medicament for reducing airway inflammation associated with, or caused by, a coronavirus infection.

36. Use of a compound of any one of claims 1-23 or a pharmaceutically acceptable salt, solvate or prodrug thereof in the preparation of a medicament for improving the ability of a subject to control a respiratory disease or condition during a coronavirus infection.

37. A compound of any one of claims 1-23 or a pharmaceutically acceptable salt, solvate or prodrug thereof, for raising an innate immune response in a subject having a coronavirus infection.

38. A compound of any one of claims 1-23 or a pharmaceutically acceptable salt, solvate or prodrug thereof, for preventing a disease caused by a coronavirus infection in a subject.

39. A compound of any one of claims 1-23 or a pharmaceutically acceptable salt, solvate or prodrug thereof, for treating and/or preventing a respiratory disease or condition associated with a coronavirus infection in a subject.

40. A compound of any one of claims 1-23 or a pharmaceutically acceptable salt, solvate or prodrug thereof, for treating and/or preventing a coronavirus infection in a subject.

41. A compound of any one of claims 1-23 or a pharmaceutically acceptable salt, solvate or prodrug thereof for reducing airway inflammation in a subject having a coronavirus infection.

42. A compound of any one of claims 1-23 or a pharmaceutically acceptable salt, solvate or prodrug thereof for controlling a respiratory disease or condition during a coronavirus infection in a subject.

43. A kit for use, or when used, in a method or use according to any one of claims 1-30, the kit comprising, consisting essentially of or consisting of:
a compound according to any one of claims 1-23; and optionally
written instructions describing the use of the compound in the method.

44. A method, use, compound or kit of any one of claims 1-43, wherein the coronavirus is from the genera Alphacoronavirus or Betacoronavirus.

45. A method, use, compound or kit of any one of claims 1-44, wherein the coronavirus is from one of the Alphacoronavirus subgroup clusters 1a and 1b.

46. A method, use, compound or kit of any one of claims 1-44, wherein the coronavirus is from one of the Betacoronavirus subgroup clusters 2a, 2b, 2c, and 2d.

47. A method, use, compound or kit of any one of claims 1-44, wherein the coronavirus is selected from the group consisting of SARS-CoV, MERS-CoV, SARS-CoV2, HCoV-NL63, HCoV-229E, HCoV-OC43 and HKU1.

48. A method, use, compound or kit of claim 46, wherein the coronavirus is SARS-CoV2.
